(19) United States
(12) Patent Application Publication
(10) Pub. No.: US 2017/0022254 A1
(43) Pub. Date: Jan. 26, 2017

(54) Novel Aldehyde Acetal Based Processes for the Manufacture of Macrocyclic Depsipeptides and New Intermediates

(71) Applicants: Murat ACEMOGLU, Basel (CH); John LOPEZ, Liestal (CH); Rolando RAVELO SILVA, Stadel (CH); Javier RUIZ RODRIGUEZ, Zurich (CH)

(72) Inventors: Murat ACEMOGLU, Basel (CH); John LOPEZ, Liestal (CH); Rolando RAVELO SILVA, Stadel (CH); Javier RUIZ RODRIGUEZ, Zurich (CH)

(73) Assignee: NOVARTIS AG, Basel (CH)

(21) Appl. No.: 15/301,831

(22) PCT Filed: Apr. 7, 2015
(86) PCT No.: PCT/IB2015/052497

§ 371 Date: Oct. 4, 2016

(30) Foreign Application Priority Data
Apr. 8, 2014 (EP) 14163939.3
Apr. 9, 2014 (EP) 14163994.8

(51) Int. Cl. C07K 11/02

(52) U.S. Cl. CPC C07K 11/02

(57) ABSTRACT

The invention relates to process for the chemical manufacture of depsipeptides of the formula (I) employing an aldehyde acetal intermediate, (Formula I) wherein the symbols have the meaning defined in the description, to new intermediates and their manufacture, as well as related invention embodiments.

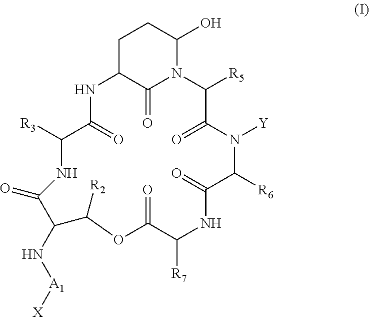

### FIELD OF THE INVENTION

The invention relates to novel processes, novel process steps and novel intermediates useful for the manufacture of macrocyclic depsipeptides.

### BACKGROUND OF THE INVENTION

Cyclic depsipeptides have numerous uses in pharmacology. As an example, the cyclic depsipeptides bearing an ahp-unit (ahp: 3-amino-6-hydroxy-piperidin-2-one) disclosed in WO2009/024527 are useful for treatment of various diseases. For example, the compound of formula II mentioned in WO2009/024527 is useful for the treatment and prevention of inflammatory and/or hyperpoliferative and pruritic skin diseases such as atopic dermatitis, psoriasis, pustular psoriasis, rosacea, keloids, hypertrophic scars, acne, Netherton's syndrome or other pruritic dermatoses such as prurigo nodularis, unspecified itch of the elderly as well as other diseases with epithelial barrier dysfunction such as aged skin.

The cyclic depsipeptides bearing an ahp-unit disclosed in WO2009/024527 can be produced via fermentation (using myxobacteria). The yield of fermentation with regard to any single of these compounds is rather low.

The chemical synthesis of cyclic depsipeptides bearing an ahp-unit in WO2012/143888, is based on approaches using a combination of solid phase and solution peptide chemistry.

A critical step in the chemical synthesis of cyclic depsipeptides bearing an ahp-unit is the formation of the ahp-substructure. This structure is mainly formed by oxidation of the open chain 2-amino-5-hydroxy-pentanoic acid moiety in the closed macrolactone ring by oxidative treatment via a labile aldehyde intermediate [Yokohama et al., Tetrahedron 61 (2005), pp. 1459-80, compound 23, Scheme 11, conversion of compound 46 to 47 and Scheme 12, conversion of compound 51 to 52; Yokohama et al., Peptide Science. 38 (2002), pp. 33-36; Yokohama et al., Tetrahedron Letters. 42 (2001), 5903-8]. Generally, the aldehyde intermediate is too instable to be isolated.

Aldehyde derivatives, such as acetals, are also known to be instable, in particular when acetal and (especially free) carboxylic acid functions are present simultaneously or under (even only slightly) acidic conditions.

There is a need to find processes that are easier in handling for the manufacture of macrolactone ring systems comprising ahp moieties.

It has been found that it is possible to replace the precursor containing the 2-amino-5-hydroxypentanoic acid moiety and use its 5-oxo-analogue in acetal form instead.

It has further also been found that it is possible to replace the precursor with the 2-amino-5-hydroxypentanoic acid building block and use its 5-oxo-analogue in acetal form, wherein the acetal serves as cleavable linker to attach a solid resin, allowing for the closure of the macrolactone ring on solid support.

The present invention thus relates to processes or methods that allow obtaining such cyclic depsipeptides with increased yield. The present invention thus relates to processes or methods that allow obtaining such cyclic depsipeptides in good purity. The present invention thus relates to processes or methods that allow obtaining such cyclic depsipeptides with a lower number of steps.

The present invention also relates to processes or methods that allow obtaining such cyclic depsipeptides in good purity and with a shorter production time, minimal use of reactors and production equipment, avoidance of diluted conditions for the macrocyclization and lower costs.

### DETAILED DESCRIPTION OF THE INVENTION

In view of the many risks, such as racemization, tautomerization and the like, in the synthesis of a complex molecule with many possible isomers, it has been possible to find a manufacturing process (Method I), comprising a mixture of solid phase peptide synthesis and reactions in solution, that allows to produce cyclic depsipeptides of formula (I) in good yield. Method I allows to produce cyclic depsipeptides of formula (I) avoiding the oxidation of a hydroxyl group in the precursor molecule. Method I also allows to produce cyclic depsipeptides of formula (IA) with the required stereoisomerical purity.

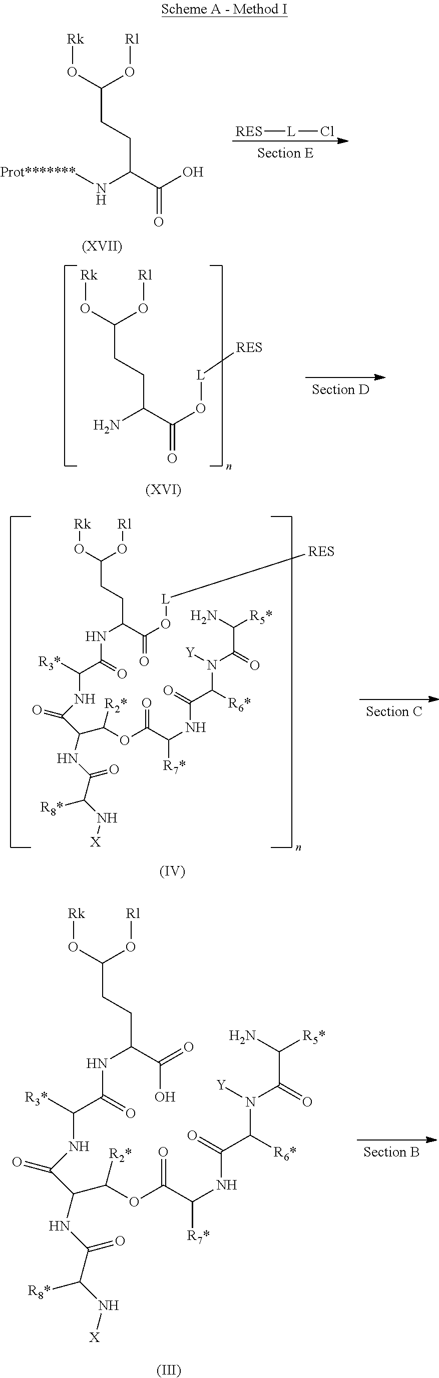

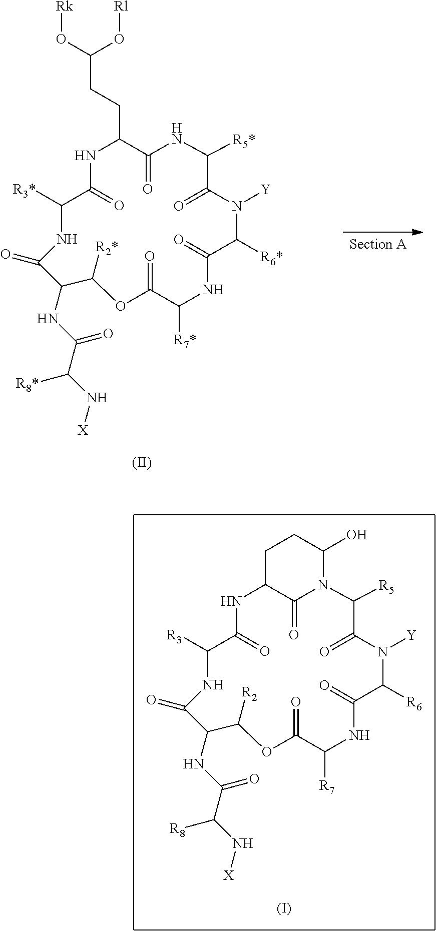

Namely, a compound of formula (XVII), or a salt thereof, is converted into a compound of formula (I), or a salt thereof, according to Method I wherein
Method I comprises
Section E to convert a compound of formula (XVII), or a salt thereof, into a compound of formula (XVI);
Section D to convert a compound of formula (XVI) into a compound of formula (IV);
Section C to convert a compound of formula (VI) into a compound of formula (III), or a salt thereof;
Section B to convert a compound of formula (III), or a salt thereof, into a compound of formula (II), or a salt thereof;
Section A to convert a compound of formula (II), or a salt thereof, into a compound of formula (I), or a salt thereof.

Sections A, B, C, D and E as such are also preferred embodiments of Method I of the present invention.

It has also been possible to find another manufacturing process (Method II), comprising a mixture of solid phase peptide synthesis and reactions in solution, that allows to produce cyclic depsipeptides of formula (I) in good yield. Method II allows to produce cyclic depsipeptides of formula (I) avoiding the oxidation of a hydroxyl group in the precursor molecule. Method II allows for the closure of the macrolactone ring on solid support. Method II also allows to produce cyclic depsipeptides of formula (IA) with the required stereoisomerical purity.

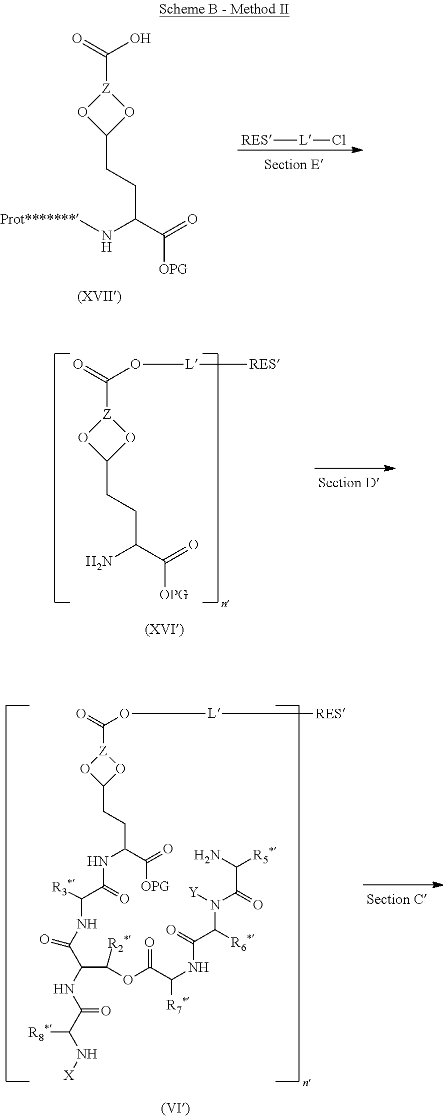

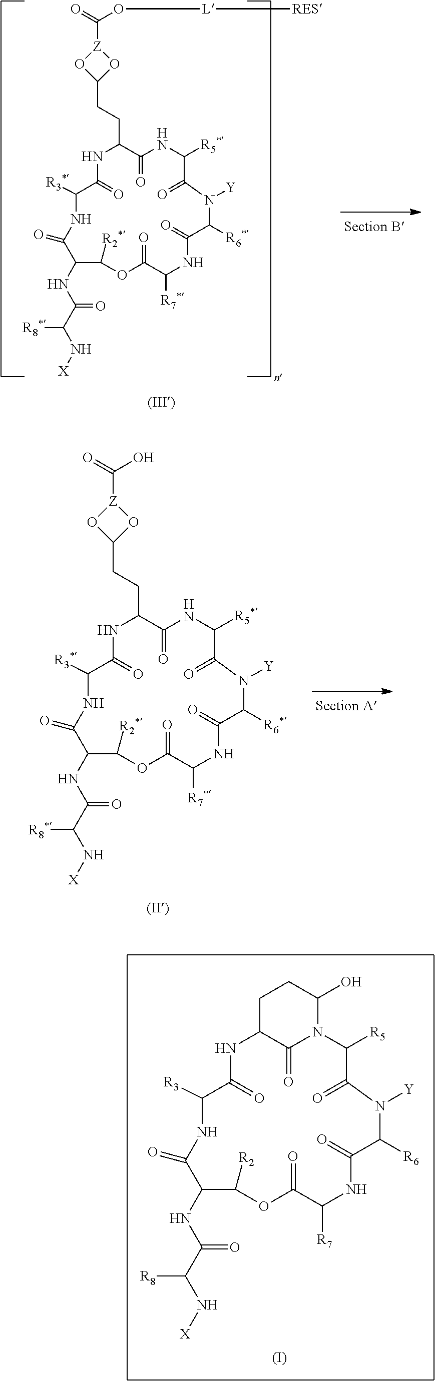

Namely, a compound of formula (XVII′), or a salt thereof, is converted into a compound of formula (I), or a salt thereof, according to Method II wherein
Method II comprises
Section E′ to convert a compound of formula (XVII′), or a salt thereof, into a compound of formula (XVI′);
Section D′ to convert a compound of formula (XVI′) into a compound of formula (IV′);
Section C′ to convert a compound of formula (IV′) into a compound of formula (III′), or a salt thereof;
Section B′ to convert a compound of formula (III′) into a compound of formula (II′), or a salt thereof;
Section A′ to convert a compound of formula (II′), or a salt thereof, into a compound of formula (I), or a salt thereof.

The invention specially relates to the processes described in each Section. The invention likewise relates, independently, to every single step described in a process sequence within the corresponding Section. Therefore, each and every single step of any process, consisting of a sequence of steps, described herein is itself a preferred embodiment of the present invention. Thus, the invention also relates to those embodiments of the process, according to which a compound obtainable as an intermediate in any step of the process is used as a starting material.

The invention likewise relates to novel starting materials which have been specifically developed for the preparation of the compounds according to the invention, to their use and to processes for their preparation.

The invention also relates to intermediates which have been specifically developed for the preparation of the compounds according to the invention, to their use and to processes for their preparation.

It is noted that in the present application usually explanations made in one Section are also applicable for other Sections, unless otherwise stated. For example, the explanations for the residue R₂ in formula (II) given in one Section also apply if formula (II) occurs in other Sections, unless otherwise stated.

It is noted that a free compound of formula (I), can be converted into a salt, a salt of a compound of formula (I) into a different salt of a compound of formula (I), or into the free compound of formula (I).

Method I, Section A: Conversion of a Compound of Formula (II), or a Salt Thereof, into a Compound of Formula (I), or a Salt Thereof

In one embodiment, the invention relates to a process for the preparation of a cyclic depsipeptide compound of formula (I), or a salt thereof,

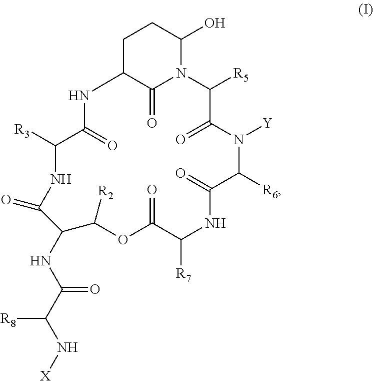

especially of the formula (IA), or a salt thereof,

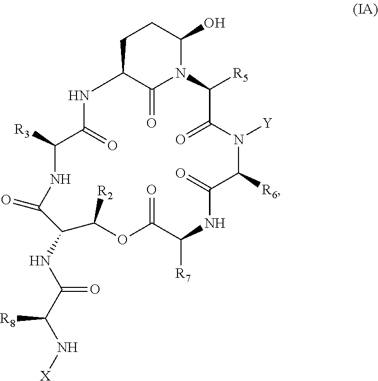

wherein
X is C₁₋₉-acyl;
R₂ is C₁₋₈-alky;
R₃ is the side chain of an alpha-amino acid;
R₅ is the side chain of an alpha-amino acid;
R₆ is the side chain of an alpha-amino acid, wherein the side chain contains a hydroxy group;
R₇ is the side chain of an alpha-amino acid;
R₈ is the side chain of an alpha-amino acid, wherein the side chain contains a terminal carboxy or carbamoyl group; and
Y is hydrogen or C₁₋₈-alkyl;
said process comprising
submitting compound of formula (II), or a salt thereof,

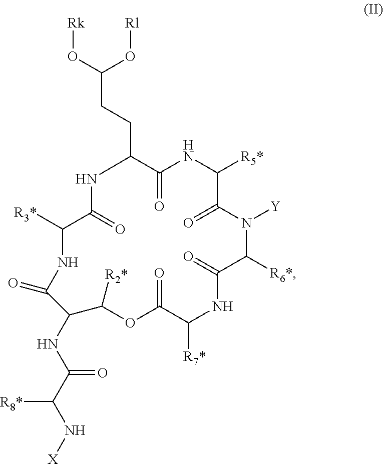

especially of the formula (IIA), or a salt thereof,

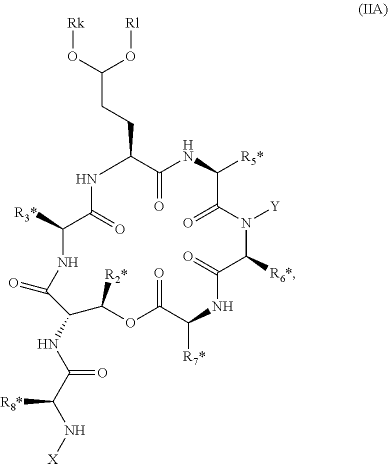

wherein the Rk and Rl are independently of each other linear or branched C₁₋₈-alkyl or benzyl or, Rk and Rl together form a linear or branched C₁₋₈-alkylene bridge, so that Rk and Rl together with the two oxygen atoms and the carbon atom to which the two oxygen atoms are bound, form a 5-7 membered ring; Y and X are as defined for a compound of formula (I) and R₂*, R₃*, R₅*, R₆*, R₇* and R₈* correspond to R₂, R₃, R₅, R₆, R₇ and R₈ in formula (I), respectively, but with the proviso that reactive functional groups on these residues are present in protected form, if they could participate in undesired side reactions,
to acetal deprotecting conditions.

Typically, acetal deprotecting conditions in Section A comprise acid catalyzed transacetalization in acetone or hydrolysis in wet solvents or in aqueous acid, especially an alpha-halo substituted alkanoic acid, such as trifluoroacetic acid or trichloroacetic acid. For example, the reaction is typically carried out in a suitable solvent in the presence of a suitable acid at a temperature range between 0° C. and 40° C. Preferably the acid used is TFA, the solvent is DCM and the reaction is carried out at rt.

Depending on the choice of protecting groups (if present) on R₂*, R₃*, R₅*, R₆*, R₇* and R₈*, Section A of Method I is a one step process, wherein all protecting groups present in a compound of formula (II), or a salt thereof, especially (IIA), or a salt thereof, are be deprotected under acetal deprotecting conditions, or Section A of Method I is a multi-step process, comprising further steps for the deprotection of protecting groups (if present) on R₂*, R₃*, R₅*, R₆*, R₇* and R₈*.

Preferably the Section A of Method I is a one step process, wherein protecting groups (if present) on R₂*, R₃*, R₅*, R₆*, R₇* and R₈* are chosen so that these protecting groups are deprotected when the compound of formula (II), or a salt thereof, especially (IIA), or a salt thereof is submitted to acetal deprotection conditions.

Preferably, for any of the processes detailed in Section A,
X is acetyl or isobutyryl;
R₂ and R₂* are methyl;
R₃ and R₃* are iso-butyl, sec-butyl, or iso-propyl;
R₅ and R₅* are benzyl, iso-butyl, sec-butyl, or iso-propyl;
R₆ is 4-hydroxybenzyl;
R₆* is 4-hydroxybenzyl, wherein the OH group is protected with a suitable protecting group;
R₇ and R₇* are iso-butyl, sec-butyl or iso-propyl;
R₈ is 2-amino-2-oxoethyl or 3-amino-3-oxopropyl;
R₈* is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl together form a —CH₂—CH₂— or —CH₂—CH₂—CH₂— bridge; and
Y is methyl.

More preferably, for any of the processes detailed in Section A,

X is isobutyryl;
R₂ and R₂* are methyl;
R₃ and R₃* are iso-butyl;
R₅ and R₅* are sec-butyl;
R₆ is 4-hydroxybenzyl;
R₆* is 4-hydroxybenzyl, wherein the OH group is protected with a suitable protecting group;
R₇ and R₇* are sec-butyl;
R₈ is 3-amino-3-oxopropyl;
R₈* is 3-amino-3-oxopropyl wherein the NH₂ group of the carbamoyl is protected with a suitable protecting group;
Rk and Rl together form a —CH₂—CH₂— bridge; and
Y is methyl.
Method I, Section B: Conversion of a Compound of Formula (III), or a Salt Thereof, into a Compound of Formula (II), or a Salt Thereof

In another embodiment of the invention relates to a process for the preparation of a compound formula (II), or a salt thereof,

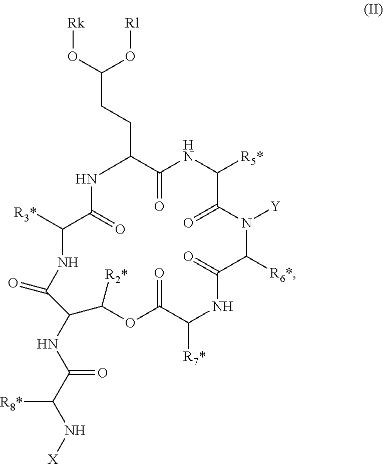

especially of the formula (IIA), or a salt thereof,

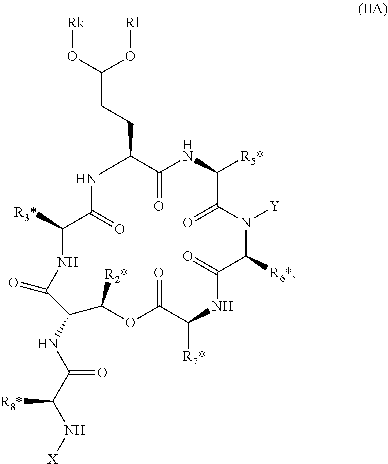

wherein Y and X are as defined for a compound of formula (I) above and Rk, Rl, R₂*, R₃*, R₅*, R₆*, R₇* and R₈* are as defined for a compound of formula (II) above,
said process comprising
submitting a linear precursor peptide of formula (III) or a salt thereof,

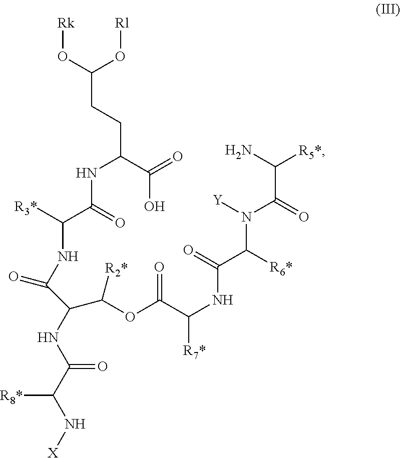

especially (IIIA), or a salt thereof,

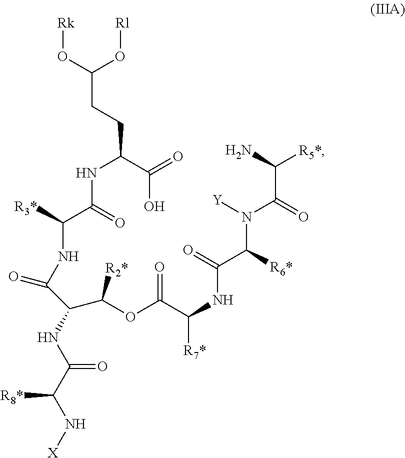

wherein Y and X are as defined for a compound of formula (I) above and Rk, Rl, R₂*, R₃*, R₅*, R₆*, R₇* and R₈* are as defined for a compound of formula (II) above, to macrolactamization conditions.

Typically, macrolactamization conditions in Section B are conditions for the coupling of a carboxy group to an amine group. The reaction is typically carried out using activating conditions for the activation of the carboxy group. Preferably, macrolactamization conditions use a coupling agent in the presence of a base in a suitable at a temperature range between 0° C. and 40° C. Preferably the coupling reagent is HATU, the base is DIPEA, or 4-DMAP, the solvent is DMF or acetonitrile and the reaction is carried out at rt.

Preferably, for any of the processes detailed in Section B,
X is acetyl or isobutyryl;
R₂* is methyl;
R₃* is iso-butyl, sec-butyl, or iso-propyl;
R₅* is benzyl, iso-butyl, sec-butyl, or iso-propyl;
R₆* is 4-hydroxybenzyl, wherein the OH group is protected with a suitable protecting group;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl together form a —CH₂—CH₂— or —CH₂—CH₂—CH₂— bridge; and
Y is methyl.

More preferably, for any of the processes detailed in Section B,

X is isobutyryl;
R₂* is methyl;
R₃* is iso-butyl;
R₅* is sec-butyl;
R₆* is 4-hydroxybenzyl, wherein the OH group is protected with a suitable protecting group;
R₇* is sec-butyl;
R₈* is 3-amino-3-oxopropyl wherein the NH₂ group of the carbamoyl is protected with a suitable protecting group;
Rk and Rl together form a —CH₂—CH₂— bridge; and
Y is methyl.
Method I, Section C: Conversion of a Compound of Formula (IV), into a Compound of Formula (III), or a Salt Thereof

In another embodiment of the invention relates to a process for the preparation of a compound formula (III) or a salt thereof,

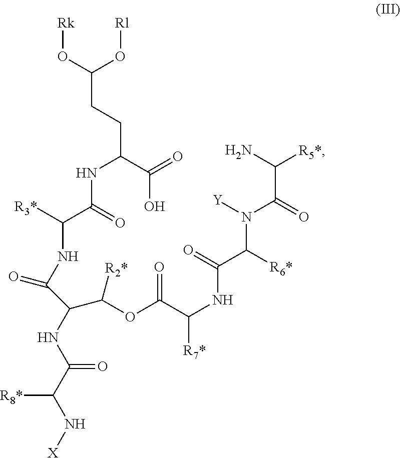

especially (IIIA), or a salt thereof,

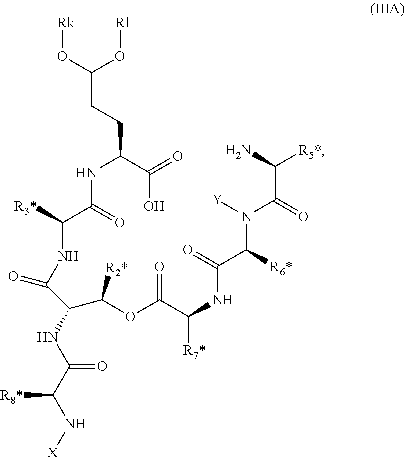

wherein Y and X are as defined for a compound of formula (I) above and Rk, Rl, R₂*, R₃*, R₅*, R₆*, R₇* and R₈* are as defined for a compound of formula (II) above, said process comprising
submitting a compound of formula (IV),

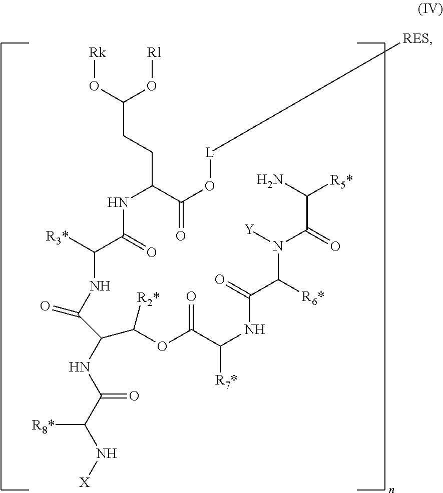

especially (IVA),

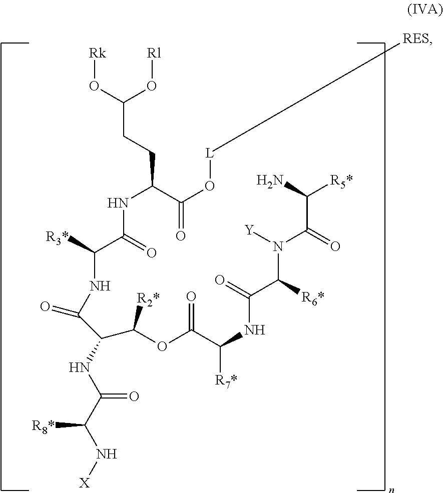

wherein Y and X are as defined for a compound of formula (I) above and Rk, Rl, R₂*, R₃*, R₅*, R₆*, R₇* and R₈* are as defined for a compound of formula (II) above,
L is a cleavable linker, RES is a solid resin and n is a natural number not including 0, to cleavage conditions.

Typically, cleavage conditions are conditions that detach a compound from solid support (RES-L-) used for SPPS. These conditions depend on the nature of the solid support, mainly on the nature of the linker L.

Typically, cleavage conditions in Section C are very mild acidic conditions for example treatment with AcOH/TFE/DCM or with HFIP in an appropriate solvent, e.g. in dichloromethane or trifluoroethanol. Preferably the cleavage reagent is HFIP, the solvent is DCM and the reaction is carried out at rt.

Preferably the cleavage conditions in Section C are chosen so that the protecting groups (if present) on R₂*, R₃*, R₅*, R₆*, R₇* and R₈* and the groups Rk and Rl are conserved.

Preferably, for any of the processes detailed in Section C,
X is acetyl or isobutyryl;
R₂* is methyl;
R₃* is iso-butyl, sec-butyl, or iso-propyl;
R₅* is benzyl, iso-butyl, sec-butyl, or iso-propyl;
R₆* is 4-hydroxybenzyl, wherein the OH group is protected with a suitable protecting group;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl together form a —CH₂—CH₂— or —CH₂—CH₂—CH₂— bridge;
Y is methyl.

More preferably, for any of the processes detailed in Section C,

X is isobutyryl;
R₂* is methyl;
R₃* is iso-butyl;
R₅* is sec-butyl;
R₆* is 4-hydroxybenzyl, wherein the OH group is protected with a suitable protecting group;
R₇* is sec-butyl;
R₈* is 3-amino-3-oxopropyl wherein the NH₂ group of the carbamoyl is protected with a suitable protecting group;
Rk and Rl together form a —CH₂—CH₂— bridge;

### L is 2Cl-trityl;

RES is divinylbenzene crosslinked polystyrene; and
Y is methyl.
Method I, Section D: Conversion of a Compound of Formula (XVI), into a Compound of Formula (IV)

In another embodiment of the invention relates to a process for the preparation of a compound formula (IV), said process comprising submitting a compound of formula (XVI) to Solid Phase Peptide Synthesis (SPPS). This process has several cycles.

Method I, Section D, Cycle 7: Conversion of a Compound of Formula (VI), into a Compound of Formula (IV)

In another embodiment of the invention relates to a process for the preparation of a compound formula (IV),

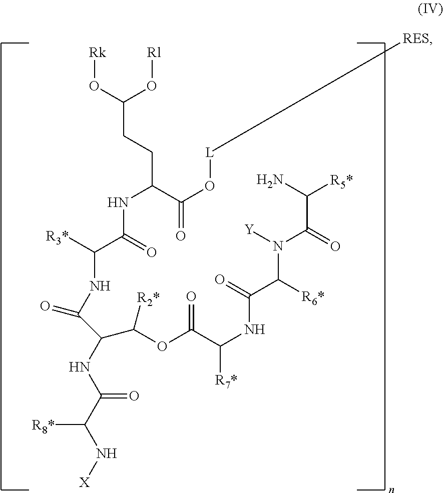

especially (IVA),

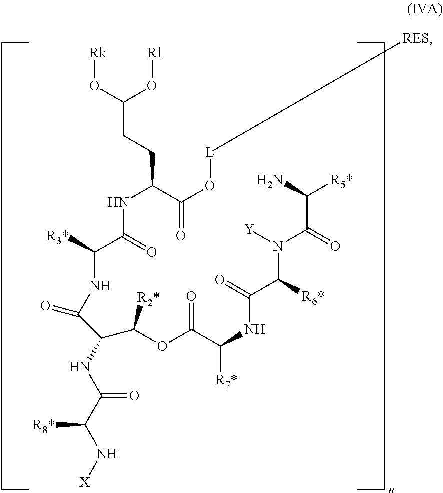

wherein Y and X are as defined for a compound of formula (I) above and Rk, Rl, R₂*, R₃*, R₅*, R₆*, R₇* and R₈* are as defined for a compound of formula (II) above,
L is a cleavable linker, RES is a solid resin and n is a natural number not including 0, said process comprising
step 1 of Method I, Section D, cycle 7 of
reacting a compound of formula (VI),

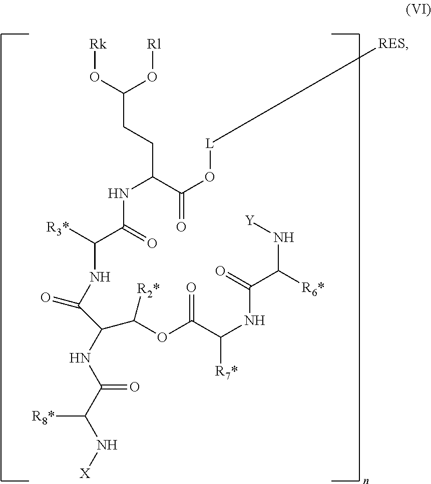

especially (VIA),

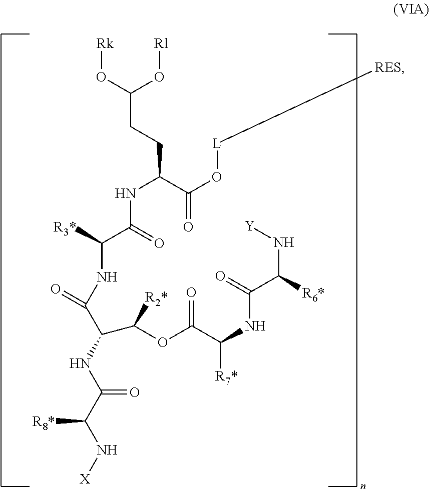

wherein Y and X are as defined for a compound of formula (I) above and Rk, Rl, R₂*, R₃*, R₆*, R₇* and R₈* are as defined for a compound of formula (II) above,
L is a cleavable linker, RES is a solid resin and n is a natural number not including 0, with a compound of formula (V)

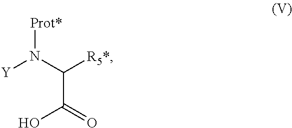

especially (VA),

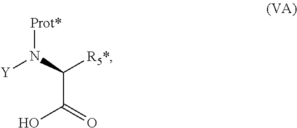

wherein R₅* is as defined for a compound of formula (II) above and Prot* is an amino protecting group
under peptide coupling conditions;
followed by step 2 of Method I, Section D, cycle 7 of
removing the protecting group Prot*
under conditions for removal of an amine protecting group.
Method I, Section D, cycle 6: Conversion of a Compound of Formula (VIII), into a Compound of Formula (VI)

In another embodiment of the invention relates to a process for the preparation of a compound formula (VI),

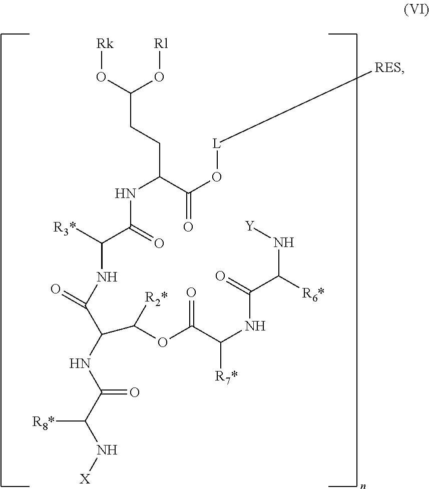

especially (VIA),

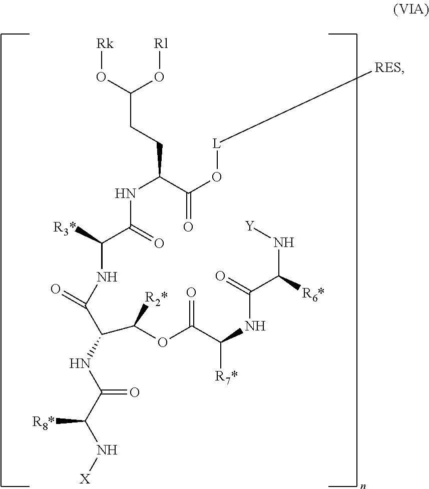

wherein Y and X are as defined for a compound of formula (I) above and Rk, Rl, R₂*, R₃*, R₆*, R₇* and R₈* are as defined for a compound of formula (II) above,
L is a cleavable linker, RES is a solid resin and n is a natural number not including 0, said process comprising
step 1 of Method I, Section D, cycle 6 of
reacting a compound of formula (VIII),

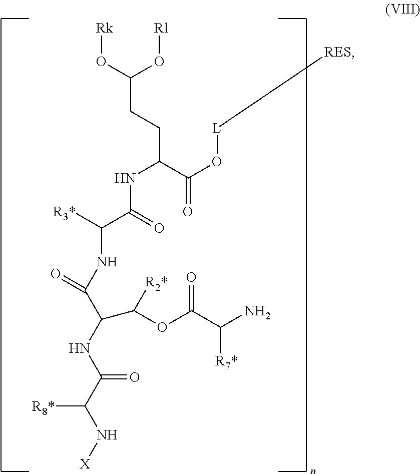

especially (VIIIA)

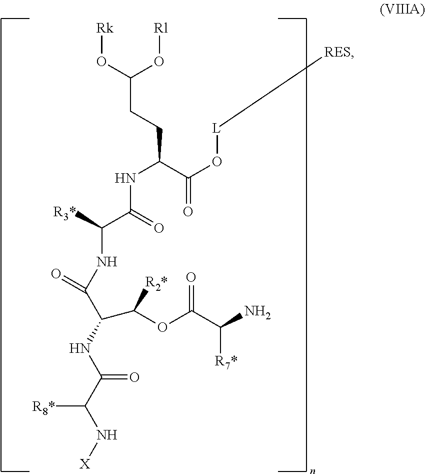

wherein X is as defined for a compound of formula (I) above and Rk, Rl, R₂*, R₃*, R₇* and R₈* are as defined for a compound of formula (II) above,
L is a cleavable linker, RES is a solid resin and n is a natural number not including 0, with a compound of formula (VII)

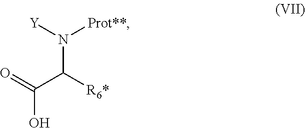

especially (VIIA),

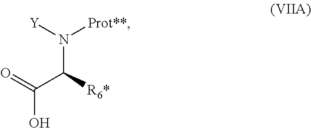

wherein Y is as defined for a compound of formula (I) above, R₆* is as defined for a compound of formula (II) above and Prot** is an amino protecting group
under peptide coupling conditions;
followed by step 2 of Method I, Section D, cycle 6 of
removing the protecting group Prot**
under conditions for removal of an amine protecting group.
Method I, Section D, Cycle 5: Conversion of a Compound of Formula (X), into a Compound of Formula (VIII)

In another embodiment of the invention relates to a process for the preparation of a compound formula (VIII),

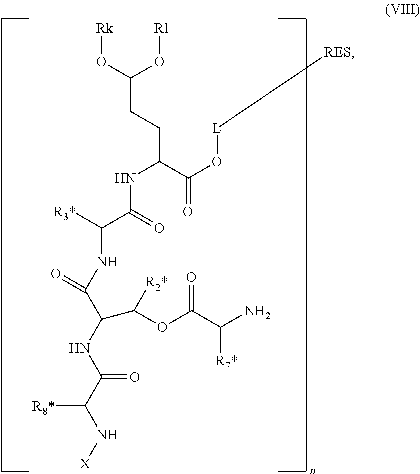

especially (VIIIA)

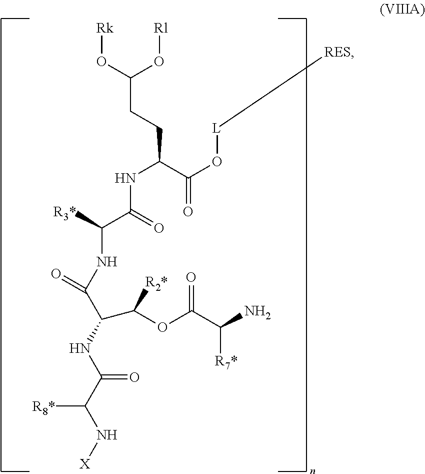

wherein X is as defined for a compound of formula (I) above and Rk, Rl, R₂*, R₃*, R₇* and R₈* are as defined for a compound of formula (II) above,
L is a cleavable linker, RES is a solid resin and n is a natural number not including 0, said process comprising
step 1 of Method I, Section D, cycle 5 of
reacting a compound of formula (X),

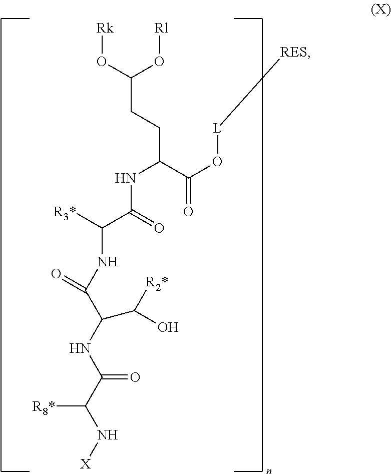

especially (XA),

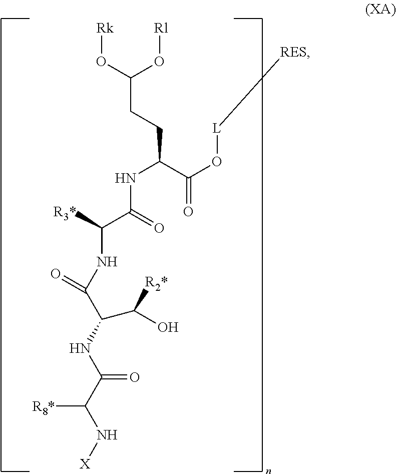

wherein X is as defined for a compound of formula (I) above, Rk, Rl, R₂*, R₃* and R₈* are as defined for a compound of formula (II) above,
L is a cleavable linker, RES is a solid resin and n is a natural number not including 0, with a compound of formula (IX)

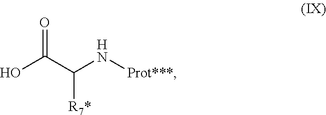

especially (IXA)

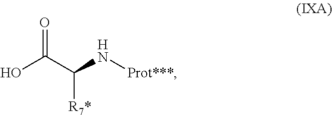

wherein R₇* is as defined for a compound of formula (II) above and Prot*** is an amino protecting group
under ester coupling conditions;
followed by step 2 of Method I, Section D, cycle 5 of
removing the protecting group Prot***
under conditions for removal of an amine protecting group.

Typically, for the ester coupling conditions mentioned in Section D cycle 5, conditions similar to peptide coupling conditions are used. Preferable ester coupling conditions use MSNT (1-(mesitylene-2-sulfonyl)-3-nitro-1,2,4-triazole) in the presence of NMI (N-methylimidazole) in DCM and the reaction is carried out at rt.

Method I, Section D, Cycle 4: Conversion of a Compound of Formula (XI), into a Compound of Formula (X)

In another embodiment of the invention relates to a process for the preparation of a compound formula (X),

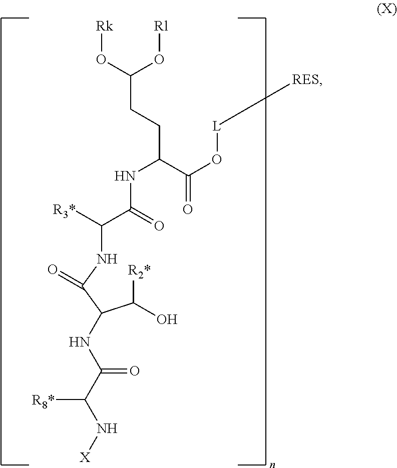

especially (XA),

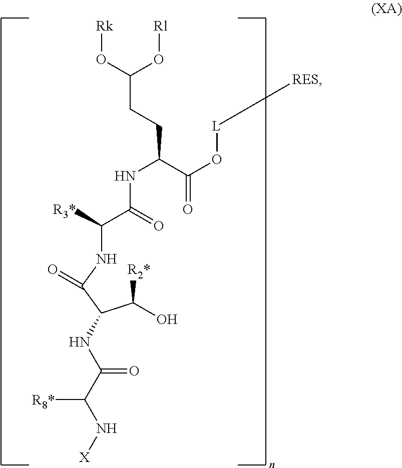

wherein X is as defined for a compound of formula (I) above, Rk, Rl, R₂*, R₃* and R₈* are as defined for a compound of formula (II) above,
L is a cleavable linker, RES is a solid resin and n is a natural number not including 0, said process comprising
step 1 of Method I, Section D, cycle 4 of
reacting a compound of formula (XI),

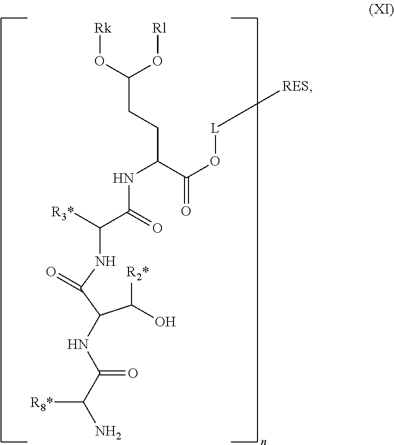

especially (XIA)

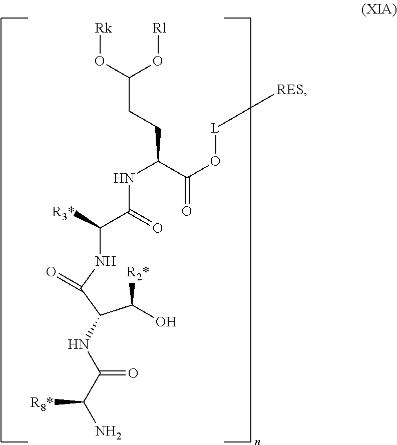

wherein Rk, Rl, R₂*, R₃* and R₈* are as defined for a compound of formula (II) above,
L is a cleavable linker, RES is a solid resin and n is a natural number not including 0, with a X—OH
wherein X is as defined for a compound of formula (I) above
under peptide coupling conditions.
Method I, Section D, Cycle 3: Conversion of a Compound of Formula (XII), into a Compound of Formula (XI)

In another embodiment of the invention relates to a process for the preparation of a compound formula (XI),

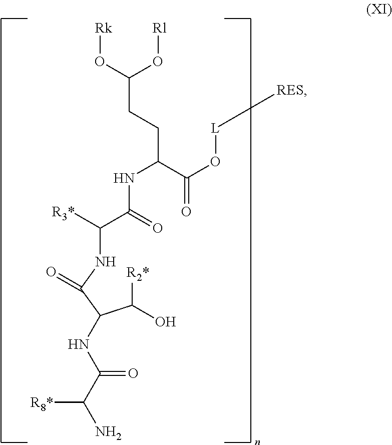

especially (XIA)

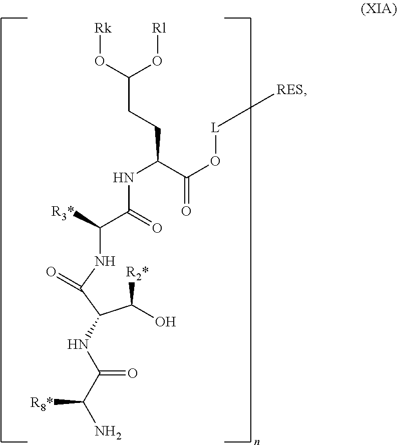

wherein Rk, Rl, R₂*, R₃* and R₈* are as defined for a compound of formula (II) above,
L is a cleavable linker, RES is a solid resin and n is a natural number not including 0, said process comprising
step 1 of Method I, Section D, cycle 3 of
reacting a compound of formula (XII),

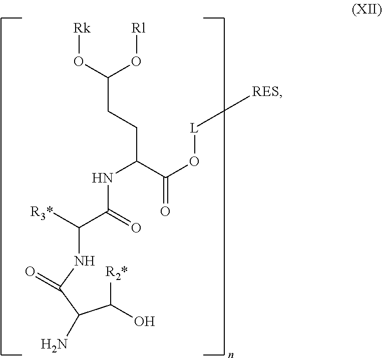

especially (XIIA)

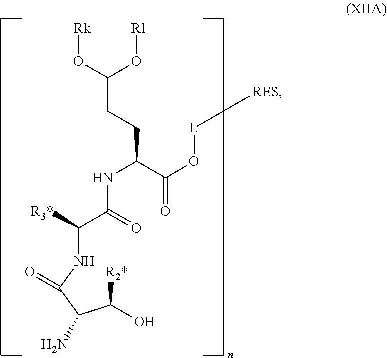

wherein Rk, Rl, R₂* and R₃* are as defined for a compound of formula (II) above,
L is a cleavable linker, RES is a solid resin and n is a natural number not including 0, with a compound of formula (XIII)

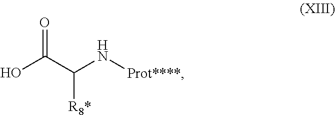

especially (XIIIA)

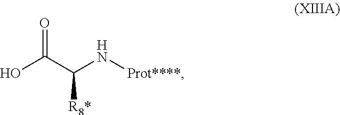

wherein R₈* is as defined for a compound of formula (II) above and Prot**** is an amino protecting group,
under peptide coupling conditions;
followed by step 2 of Method I, Section D, cycle 3 of
removing the protecting group Prot****
under conditions for removal of an amine protecting group.
Method I, Section D, Cycle 2: Conversion of a Compound of Formula (XIV), into a Compound of Formula (XII)

In another embodiment of the invention relates to a process for the preparation of a compound formula (XII),

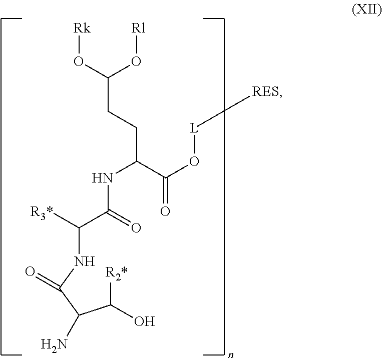

especially (XIIA)

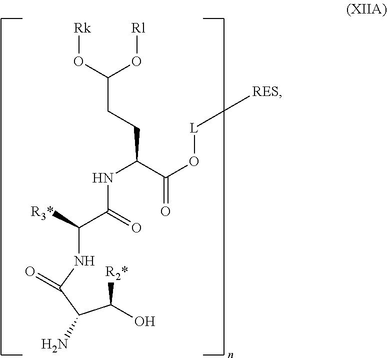

wherein Rk, Rl, R₂* and R₃* are as defined for a compound of formula (II) above,
L is a cleavable linker, RES is a solid resin and n is a natural number not including 0, said process comprising
step 1 of Method I, Section D, cycle 2 of
reacting a compound of formula (XIV),

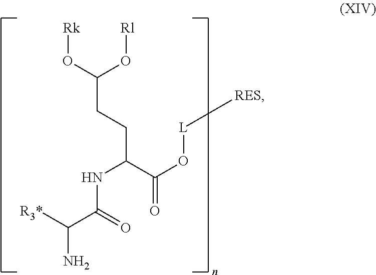

especially (XIVA)

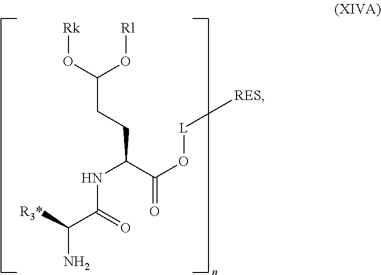

wherein Rk, Rl, and R₃* are as defined for a compound of formula (II) above,
L is a cleavable linker, RES is a solid resin and n is a natural number not including 0, with a compound of formula (XV)

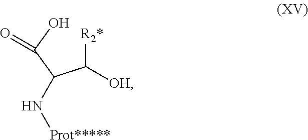

especially (XVA)

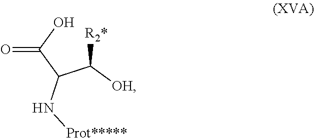

wherein R₂* is as defined for a compound of formula (II) above and Prot***** is an amino protecting group,
under peptide coupling conditions;
followed by step 2 of Method I, Section D, cycle 2 of
removing the protecting group Prot*****
under conditions for removal of an amine protecting group.
Method I, Section D, Cycle 1: Conversion of a Compound of Formula (XVI), into a Compound of Formula (XIV)

In another embodiment of the invention relates to a process for the preparation of a compound formula (XIV),

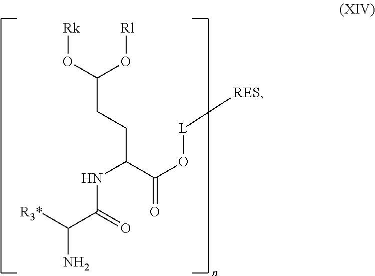

especially (XIVA)

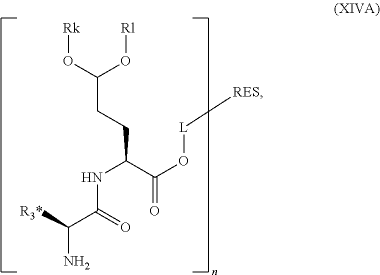

wherein Rk, Rl, and R₃* are as defined for a compound of formula (II) above,
L is a cleavable linker, RES is a solid resin and n is a natural number not including 0, said process comprising
step 1 of Method I, Section D, cycle 1 of
reacting a compound of formula (XVI),

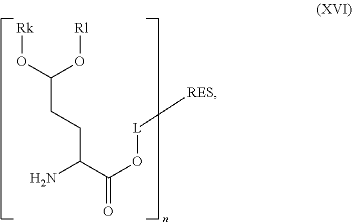

especially (XVIA)

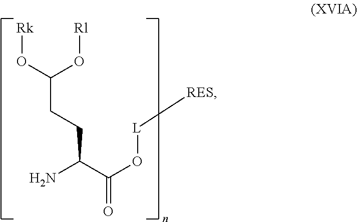

wherein Rk and Rl are as defined for a compound of formula (II) above,
L is a cleavable linker, RES is a solid resin, and n is a natural number not including 0, with a compound of formula (XVII)

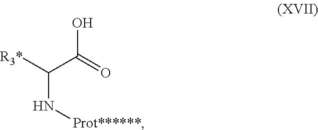

especially (XVIIA)

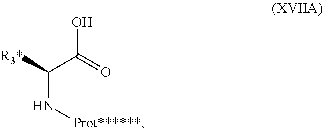

wherein R₃* is as defined for a compound of formula (II) above and Prot****** is an amino protecting group,
under peptide coupling conditions;
followed by step 2 of Method I, Section D, cycle 1 of
removing the protecting group Prot******
under conditions for removal of an amine protecting group.

Typically, peptide coupling conditions mentioned in Section D are carried out using a coupling agent, preferably in the presence of a mild base, typically in the presence of an appropriate solvent or solvent mixture, e.g. an N,N dialkylformamide, such as dimethylformamide, a halogenated hydrocarbon, e.g. dichloromethane, N-alkylpyrro-lidones, such as N-methylpyrrolidone, nitriles, e.g. acetonitrile, ethers, such as dioxane or tetrahydrofurane, or aromatic hydrocarbons, e.g. toluene, or mixtures of two or more, where, provided an excess of coupling agent is present, also water may be present. The temperatures may be ambient temperature or lower or higher, e.g. in the range from −20° C. to 50° C. Preferable peptide coupling conditions use HATU (2-(1H-7-azabenzotriazol-1-yl)-1,1,3,3-tetramethyluronium hexafluorophosphate methanaminium) in the presence of DIEPA (N,N-diisopropylethylamine) or Oxyma (ethyl 2-cyano-2-(hydroxyimino)acetate)/DICI (diisopropylcarbodiimide), in DMF and the reaction is carried out at rt.

Typically, conditions for removal of an amine protecting group in Section D are basic conditions. The reaction is typically carried out in a suitable solvent, e.g. an N,N dialkylformamide, such as dimethylformamide, a halogenated hydrocarbon, e.g. dichloromethane, alkanols, such as ethanol, propanol or isopropanol, nitriles, e.g. acetonitrile, or aromatic hydrocarbons, e.g. toluene, or mixtures of two or more, also water may be present, in the presence of a suitable mild base, such as piperidine, morpholine, dicyclohexylamine, p-dimethylamino-pyridine, diisopropylamine, piperazine, tris-(2-aminoethyl)amine or 4-methylpiperidine in an appropriate solvent, e.g. N,N-dimethylformamide, methylene chloride, at a temperature range between 0° C. and 40° C. Preferably the base used is piperidine, the solvent is DMF and the reaction is carried out at rt.

Preferably the peptide coupling conditions and conditions for removal of an amine protecting group in Section D are chosen so that the protecting groups (if present) on R₂*, R₃*, R₅*, R₆*, R₇* and R₈* (where present) and the groups Rk and Rl are conserved and ester groups (where present) are not hydrolyzed.

Preferably, for any of the processes detailed in Section D,
X (where present) is acetyl or isobutyryl;
R₂* (where present) is methyl;
R₃* (where present) is iso-butyl, sec-butyl, or iso-propyl;
R₅* (where present) is benzyl, iso-butyl, sec-butyl, or iso-propyl;
R₆* (where present) is 4-hydroxybenzyl, wherein the OH group is protected with a suitable protecting group;
R₇* (where present) is iso-butyl, sec-butyl or iso-propyl;
R₈* (where present) is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl together form a —CH₂—CH₂— or —CH₂—CH₂—CH₂— bridge;
Y (where present) is methyl; and
Prot*, Prot**, Prot***, Prot****, Prot***** and Prot****** (where present) are selected from fluoren-9-ylmethoxycarbonyl (Fmoc); 2-(2′ or 4′-pyridyl)ethoxycarbonyl and 2,2-bis(4′nitrophenyl)ethoxycarbonyl.

More preferably, for any of the processes detailed in Section D,

X (where present) is isobutyryl;
R₂* (where present) is methyl;
R₃* (where present) is iso-butyl;
R₅* (where present) is sec-butyl;
R₆* (where present) is 4-hydroxybenzyl, wherein the OH group is protected with a suitable protecting group;
R₇* (where present) is sec-butyl;
R₈* (where present) is 3-amino-3-oxopropyl wherein the NH₂ group of the carbamoyl is protected with a suitable protecting group;
Rk and Rl together form a —CH₂—CH₂— bridge;

### L is 2Cl-trityl;

RES is divinylbenzene crosslinked polystyrene; and
Y (where present) is methyl; and
Prot*, Prot**, Prot***, Prot****, Prot***** and Prot****** (where present) are Fmoc.
Method I, Section E: Conversion of a Compound of Formula (XVII), or a Salt Thereof, into a Compound of Formula (XVI)

In another embodiment of the invention relates to a process for the preparation of a compound formula (XVI)

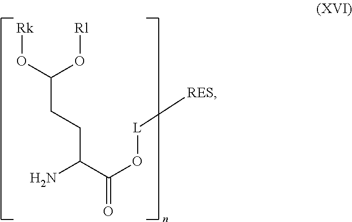

especially (XVIA)

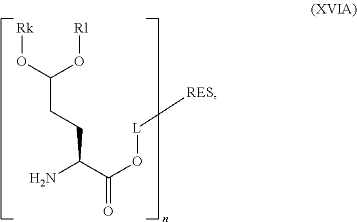

wherein Rk and Rl are as defined for a compound of formula (II) above,
L is a cleavable linker, RES is a solid resin, and n is a natural number not including 0, said process comprising
step 1 of Method I, Section E, of
submitting a compound of formula (XVII), or a salt thereof,

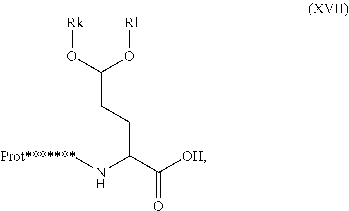

especially (XVIIA), or a salt thereof,

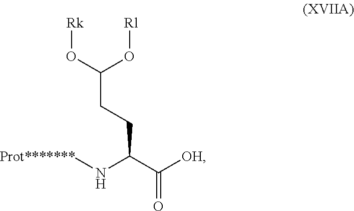

wherein Rk and Rl are as defined for a compound of formula (II) above and Prot******* is an amino protecting group,
to conditions for loading to solid support;
followed by step 2 of Method I, Section E, of
removing the protecting group Prot*******
under conditions for removal of an amine protecting group.

Typically, conditions for loading to solid support (RES-L-) in Section E depend on the nature of the solid support, mainly on the nature of the linker L. For example the unloaded solid support is suspended in an appropriate solvent, such as a dialkyl acid amide, e.g. dimethylformamide, an alcohol, such as ethanol, propanol or isopropanol or dichloromethane and reacted with the carboxyl group containing compound to be loaded to solid support. Preferably, for the loading to chloro-(2′chloro)trityl-polystyrene resin (RES-L-CI, wherein RES is divinylbenzene crosslinked polystyrene and L is 2Cl-trityl), the resin is suspended in an appropriate solvent, e.g. dichloromethane and reacted with the carboxyl group containing compound to be loaded in the presence of a base, e.g. a tertiary amino base, such as DIPEA.

Typically, conditions for removal of an amine protecting group in Section E are mild basic conditions. The reaction is typically carried out in a suitable solvent, such as DMF or DCM in the presence of a suitable mild base, such as piperidine, morpholine, dicyclohexylamine, p-dimethylamino-pyridine, diisopropylamine, piperazine, tris-(2-aminoethyl)amine at a temperature range between 0° C. and 40° C. Preferably the base used is piperidine, the solvent is DMF and the reaction is carried out at rt.

Preferably, for any of the processes detailed in Section E,
Rk and Rl are each independently of each other C₁₋₈-alkyl or benzyl, or Rk and Rl together form a —CH₂—CH₂— or —CH₂—CH₂—CH₂— bridge; and
Prot******* is selected from fluoren-9-ylmethoxycarbonyl (Fmoc); 2-(2′ or 4′-pyridyl)ethoxycarbonyl and 2,2-bis(4′nitrophenyl)ethoxycarbonyl.

More preferably, for any of the processes detailed in Section E,

Rk and Rl together form a —CH₂—CH₂— bridge;

### L is 2Cl-trityl;

RES is divinylbenzene crosslinked polystyrene; and

### Prot******* is Fmoc.

In another embodiment, the present invention relates to process for the preparation of a cyclic depsipeptide compound of formula (I), or a salt thereof, especially of the formula (IA), or a salt thereof, comprising
Method I, Section A as described herein;
Method I, Section B as described herein;
Method I, Section C as described herein;
Method I, Section D as described herein; and
Method I, Section E as described herein.

In another embodiment, the present invention relates to process for the preparation of a cyclic depsipeptide compound of formula (I), or a salt thereof, especially of the formula (IA), or a salt thereof, comprising
Method I, Section A as described herein;
Method I, Section B as described herein;
Method I, Section C as described herein; and
Method I, Section D as described herein.

In another embodiment, the present invention relates to process for the preparation of a cyclic depsipeptide compound of formula (I), or a salt thereof, especially of the formula (IA), or a salt thereof, comprising
Method I, Section A as described herein;
Method I, Section B as described herein; and
Method I, Section C as described herein.

In another embodiment, the present invention relates to process for the preparation of a cyclic depsipeptide compound of formula (I), or a salt thereof, especially of the formula (IA), or a salt thereof, comprising
Method I, Section A as described herein; and
Method I, Section B as described herein.

In another embodiment, the present invention relates to process for the preparation of a compound of formula (II), or a salt thereof, especially of the formula (IIA), or a salt thereof, comprising
Method I, Section B as described herein;
Method I, Section C as described herein;
Method I, Section D as described herein; and
Method I, Section E as described herein.

In another embodiment, the present invention relates to process for the preparation of a compound of formula (II), or a salt thereof, especially of the formula (IIA), or a salt thereof, comprising
Method I, Section B as described herein;
Method I, Section C as described herein; and
Method I, Section D as described herein.

In another embodiment, the present invention relates to process for the preparation of a compound of formula (II), or a salt thereof, especially of the formula (IIA), or a salt thereof, comprising
Method I, Section B as described herein; and
Method I, Section C as described herein.

In another embodiment, the present invention relates to process for the preparation of a compound of formula (III), or a salt thereof, especially of the formula (IIIA), or a salt thereof, comprising
Method I, Section C as described herein;
Method I, Section D as described herein; and
Method I, Section E as described herein.

In another embodiment, the present invention relates to process for the preparation of a compound of formula (III), or a salt thereof, especially of the formula (IIIA), or a salt thereof, comprising
Method I, Section C as described herein; and
Method I, Section D as described herein.

In another embodiment, the present invention relates to process for the preparation of a compound of formula (IV), especially of the formula (IVA), comprising
Method I, Section D as described herein; and
Method I, Section E as described herein.

In a further embodiment, the invention relates to a compound of formula (II), or a salt thereof,

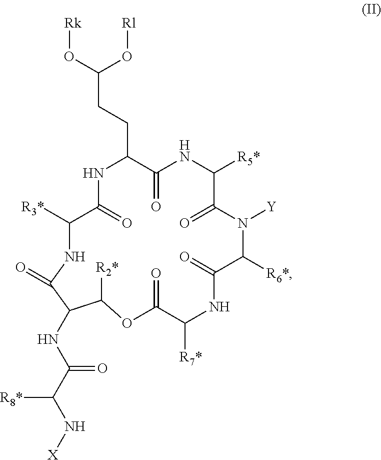

especially of the formula (IIA), or a salt thereof,

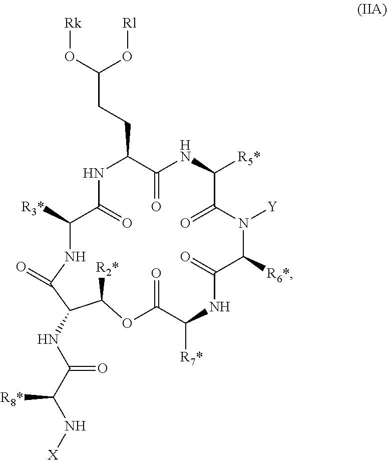

wherein the Rk and Rl are independently of each other linear or branched C₁₋₈-alkyl or benzyl or, Rk and Rl together form a linear or branched C₁₋₈-alkylene bridge, so that Rk and Rl together with the two oxygen atoms and the carbon atom to which the two oxygen atoms are bound, form a 5-7 membered ring; Y and X are as defined for a compound of formula (I) and R₂*, R₃*, R₅*, R₆*, R₇* and R₈* correspond to R₂, R₃, R₅, R₆, R₇ and R₈ in formula (I), respectively, but with the proviso that reactive functional groups on these residues are present in protected form, if they could participate in undesired side reactions.

In a preferred embodiment, the invention relates to a compound of formula (II), or a salt thereof, especially of the formula (IIA), or a salt thereof, wherein

X is acetyl or isobutyryl;
R₂* is methyl;
R₃* is iso-butyl, sec-butyl, or iso-propyl;
R₅* is benzyl, iso-butyl, sec-butyl, or iso-propyl;
R₆* is 4-hydroxybenzyl, wherein the OH group is protected with a suitable protecting group;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group; and
Y is methyl.

In a more preferred embodiment, the invention relates to a compound of formula (II), or a salt thereof, especially of the formula (IIA), or a salt thereof, wherein

X is isobutyryl;
R₂* is methyl;
R₃* is iso-butyl;
R₅* is sec-butyl;
R₆* is 4-hydroxybenzyl, wherein the OH group is protected with a suitable protecting group;
R₇* is sec-butyl;
R₈* is 3-amino-3-oxopropyl wherein the NH₂ group of the carbamoyl is protected with a suitable protecting group; and
Y is methyl.

In another embodiment, the present invention relates to the use of the compound of formula (II), or salt thereof, especially of the formula (IIA), or a salt thereof, for the synthesis of a compound of formula (I), or salt thereof, especially of the formula (IA), or a salt thereof.

In a further embodiment, the invention relates to a compound of formula (III), or a salt thereof,

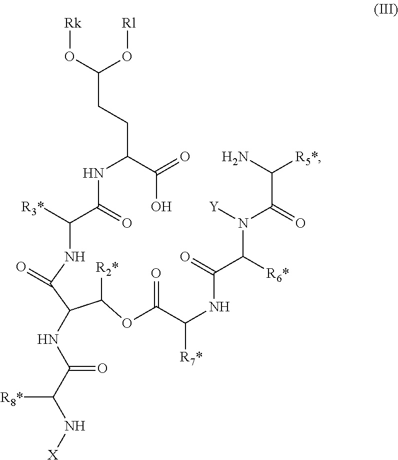

especially (IIIA), or a salt thereof

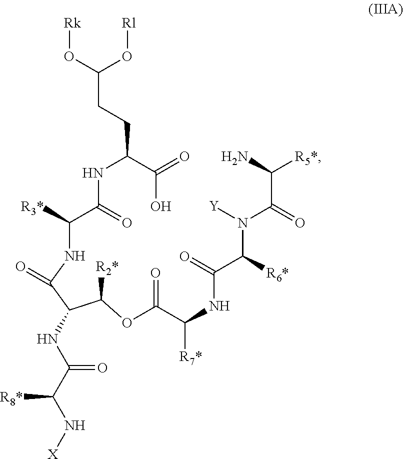

wherein Y and X are as defined for a compound of formula (I) above and Rk, Rl, R₂*, R₃*, R₅*, R₆*, R₇* and R₈* are as defined for a compound of formula (II) above.

In a preferred embodiment, the invention relates to a compound of formula (III), or a salt thereof, especially of the formula (IIIA), or a salt thereof, wherein

X is acetyl or isobutyryl;
R₂* is methyl;
R₃* is iso-butyl, sec-butyl, or iso-propyl;
R₅* is benzyl, iso-butyl, sec-butyl, or iso-propyl;
R₆* is 4-hydroxybenzyl, wherein the OH group is protected with a suitable protecting group;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group; and
Y is methyl.

In a more preferred embodiment, the invention relates to a compound of formula (III), or a salt thereof, especially of the formula (IIIA), or a salt thereof, wherein

X is isobutyryl;
R₂* is methyl;
R₃* is iso-butyl;
R₅* is sec-butyl;
R₆* is 4-hydroxybenzyl, wherein the OH group is protected with a suitable protecting group;
R₇* is sec-butyl;
R₈* is 3-amino-3-oxopropyl wherein the NH₂ group of the carbamoyl is protected with a suitable protecting group; and
Y is methyl.

In another embodiment, the present invention relates to the use of the compound of formula (III), or salt thereof, especially of the formula (IIIA), or a salt thereof, for the synthesis of a compound of formula (I), or salt thereof, especially of the formula (IA), or a salt thereof.

In a further embodiment, the invention relates to a compound of formula (IV),

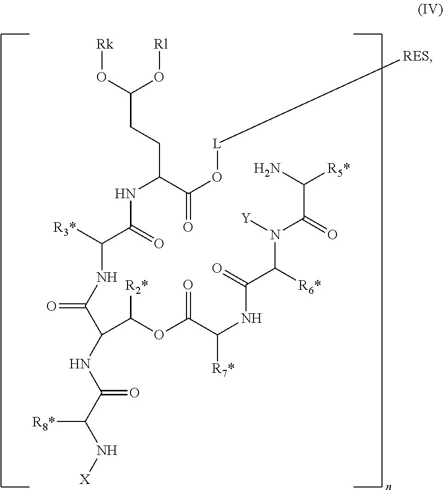

especially (IVA),

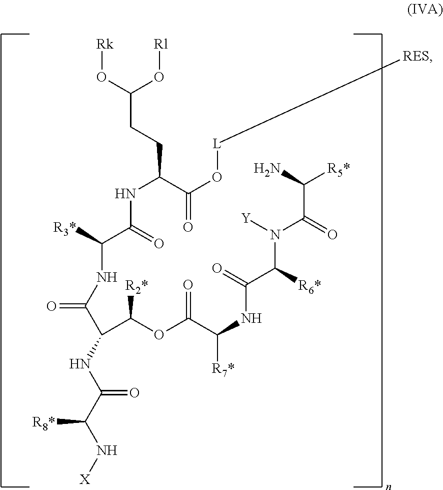

wherein Y and X are as defined for a compound of formula (I) above and Rk, Rl, R₂*, R₃*, R₅*, R₆*, R₇* and R₈* are as defined for a compound of formula (II) above,
L is a cleavable linker, RES is a solid resin and n is a natural number not including 0.

In a preferred embodiment, the invention relates to a compound of formula (IV), especially of the formula (IVA), wherein
X is acetyl or isobutyryl;
R₂* is methyl;
R₃* is iso-butyl, sec-butyl, or iso-propyl;
R₅* is benzyl, iso-butyl, sec-butyl, or iso-propyl;
R₆* is 4-hydroxybenzyl, wherein the OH group is protected with a suitable protecting group;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl
together form a —CH₂—CH₂— or —CH₂—CH₂—CH₂— bridge; and
Y is methyl.

In a more preferred embodiment, the invention relates to a compound of formula (IV), especially of the formula (IVA), wherein

X is isobutyryl;
R₂* is methyl;
R₃* is iso-butyl;
R₅* is sec-butyl;
R₆* is 4-hydroxybenzyl, wherein the OH group is protected with a suitable protecting group;
R₇* is sec-butyl;
R₈* is 3-amino-3-oxopropyl wherein the NH₂ group of the carbamoyl is protected with a suitable protecting group;
Rk and Rl together form a —CH₂—CH₂— bridge;

### L is 2Cl-trityl;

RES is divinylbenzene crosslinked polystyrene; and
Y is methyl.

In another embodiment, the present invention relates to the use of the compound of formula (IV), or salt thereof, especially of the formula (IVA), or a salt thereof, for the synthesis of a compound of formula (I), or salt thereof, especially of the formula (IA), or a salt thereof.

In a further embodiment, the invention relates to a compound of formula (VI),

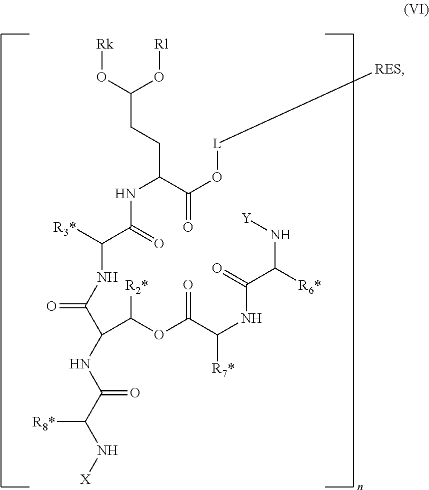

especially (VIA),

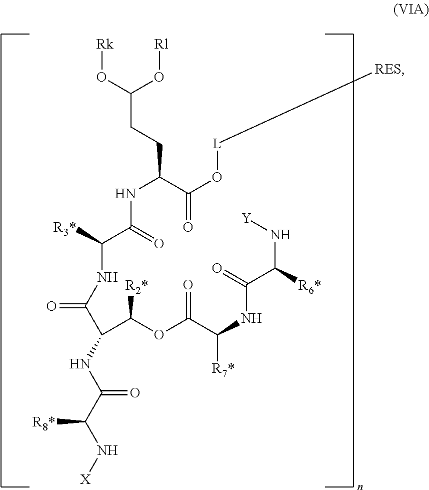

wherein Y and X are as defined for a compound of formula (I) above and Rk, Rl, R₂*, R₃*, R₆*, R₇* and R₈* are as defined for a compound of formula (II) above,
L is a cleavable linker, RES is a solid resin and n is a natural number not including 0.

In a preferred embodiment, the invention relates to a compound of formula (VI), especially of the formula (VIA), wherein
X is acetyl or isobutyryl;
R₂* is methyl;
R₃* is iso-butyl, sec-butyl, or iso-propyl;
R₆* is 4-hydroxybenzyl, wherein the OH group is protected with a suitable protecting group;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl together form a —CH₂—CH₂— or —CH₂—CH₂—CH₂— bridge; and
Y is methyl.

In a more preferred embodiment, the invention relates to a compound of formula (VI), especially of the formula (VIA), wherein

X is isobutyryl;
R₂* is methyl;
R₃* is iso-butyl;
R₆* is 4-hydroxybenzyl, wherein the OH group is protected with a suitable protecting group;
R₇* is sec-butyl;
R₈* is 3-amino-3-oxopropyl wherein the NH₂ group of the carbamoyl is protected with a suitable protecting group;
Rk and Rl together form a —CH₂—CH₂— bridge;

### L is 2Cl-trityl;

RES is divinylbenzene crosslinked polystyrene; and
Y is methyl.

In another embodiment, the present invention relates to the use of the compound of formula (VI), or salt thereof, especially of the formula (VIA), or a salt thereof, for the synthesis of a compound of formula (I), or salt thereof, especially of the formula (IA), or a salt thereof.

In a further embodiment, the invention relates to a compound of formula (VIII),

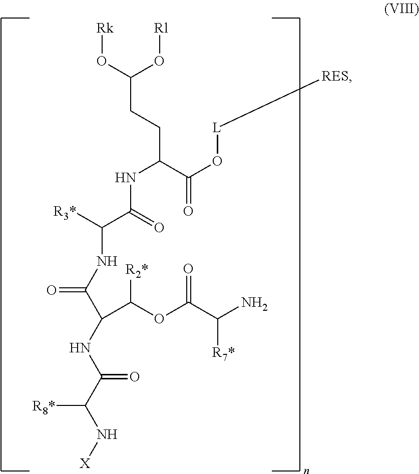

especially (VIIIA)

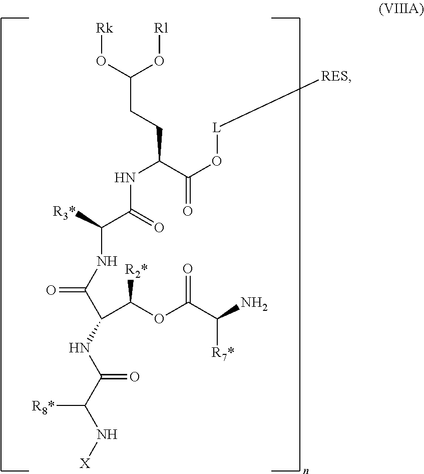

wherein X is as defined for a compound of formula (I) above and Rk, Rl, R₂*, R₃*, R₇* and R₈* are as defined for a compound of formula (II) above,
L is a cleavable linker, RES is a solid resin and n is a natural number not including 0.

In a preferred embodiment, the invention relates to a compound of formula (VIII), especially of the formula (VIIIA), wherein
X is acetyl or isobutyryl;
R₂* is methyl;
R₃* is iso-butyl, sec-butyl, or iso-propyl;
R₇* is iso-butyl, sec-butyl or iso-propyl;
R₈* is 2-amino-2-oxoethyl or 3-amino-3-oxopropyl wherein the NH₂ group of the carbamoyl is protected with a suitable protecting group;
Rk and Rl are each independently of each other C₁₋₈-alkyl or benzyl, or Rk and Rl together form a —CH₂—CH₂— or —CH₂—CH₂—CH₂— bridge; and
polymethacrylamide.

In a more preferred embodiment, the invention relates to a compound of formula (VIII), especially of the formula (VIIIA), wherein

X is isobutyryl;
R₂* is methyl;
R₃* is iso-butyl;
R₇* is sec-butyl;
R₈* is 3-amino-3-oxopropyl wherein the NH₂ group of the carbamoyl is protected with a suitable protecting group;
Rk and Rl together form a —CH₂—CH₂— bridge;

### L is 2Cl-trityl; and

RES is divinylbenzene crosslinked polystyrene.

In another embodiment, the present invention relates to the use of the compound of formula (VIII), or salt thereof, especially of the formula (VIIIA), or a salt thereof, for the synthesis of a compound of formula (I), or salt thereof, especially of the formula (IA), or a salt thereof.

In a further embodiment, the invention relates to a compound of formula (X),

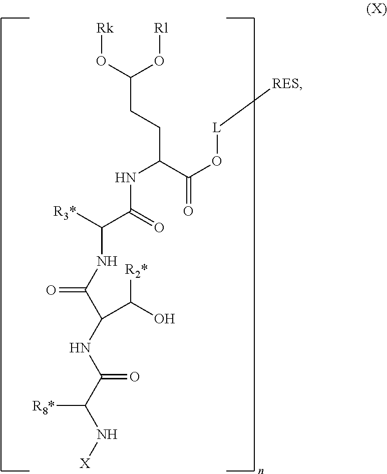

especially (XA),

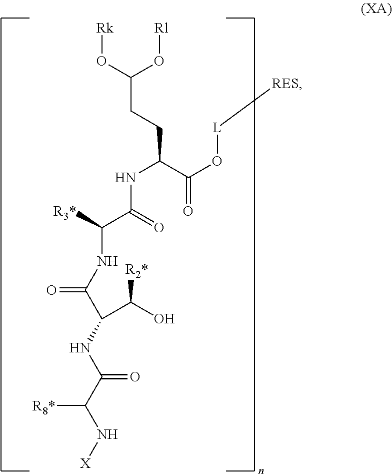

wherein X is as defined for a compound of formula (I) above and Rk, Rl, R₂*, R₃* and R₈* are as defined for a compound of formula (II) above,
L is a cleavable linker, RES is a solid resin and n is a natural number not including 0.

In a preferred embodiment, the invention relates to a compound of formula (X), especially of the formula (XA), wherein
X is acetyl or isobutyryl;
R₂* is methyl;
R₃* is iso-butyl, sec-butyl, or iso-propyl;
R₈* is 2-amino-2-oxoethyl or 3-amino-3-oxopropyl wherein the NH₂ group of the carbamoyl is protected with a suitable protecting group; and
Rk and Rl are each independently of each other C₁₋₈-alkyl or benzyl, or Rk and Rl together form a —CH₂—CH₂— or —CH₂—CH₂—CH₂— bridge.

In a more preferred embodiment, the invention relates to a compound of formula (X), especially of the formula (XA), wherein

X is isobutyryl;
R₂* is methyl;
R₃* is iso-butyl;
R₈* is 3-amino-3-oxopropyl wherein the NH₂ group of the carbamoyl is protected with a suitable protecting group;
Rk and Rl together form a —CH₂—CH₂— bridge;

### L is 2Cl-trityl; and

RES is divinylbenzene crosslinked polystyrene.

In another embodiment, the present invention relates to the use of the compound of formula (X), or salt thereof, especially of the formula (XA), or a salt thereof, for the synthesis of a compound of formula (I), or salt thereof, especially of the formula (IA), or a salt thereof.

In a further embodiment, the invention relates to a compound of formula (XI),

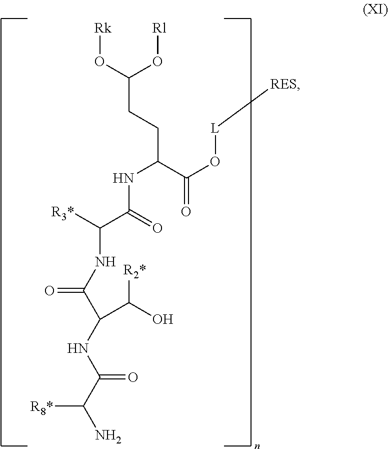

especially (XIA)

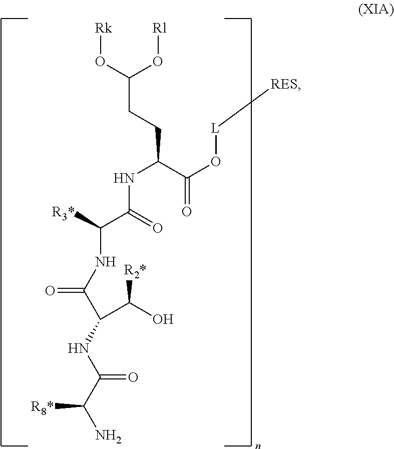

wherein Rk, Rl, R₂*, R₃* and R₈* are as defined for a compound of formula (II) above, L is a cleavable linker, RES is a solid resin and n is a natural number not including 0.

In a preferred embodiment, the invention relates to a compound of formula (XI), especially of the formula (XIA), wherein
R₂* is methyl;
R₃* is iso-butyl, sec-butyl, or iso-propyl;
R₈* is 2-amino-2-oxoethyl or 3-amino-3-oxopropyl wherein the NH₂ group of the carbamoyl is protected with a suitable protecting group; and
Rk and Rl are each independently of each other C₁₋₈-alkyl or benzyl, or Rk and Rl together form a —CH₂—CH₂— or —CH₂—CH₂—CH₂— bridge.

In a more preferred embodiment, the invention relates to a compound of formula (XI), especially of the formula (XIA), wherein

R₂* is methyl;
R₃* is iso-butyl;
R₈* is 3-amino-3-oxopropyl wherein the NH₂ group of the carbamoyl is protected with a suitable protecting group;
Rk and Rl together form a —CH₂—CH₂— bridge;

### L is 2Cl-trityl; and

RES is divinylbenzene crosslinked polystyrene.

In another embodiment, the present invention relates to the use of the compound of formula (XI), or salt thereof, especially of the formula (XIA), or a salt thereof, for the synthesis of a compound of formula (I), or salt thereof, especially of the formula (IA), or a salt thereof.

In a further embodiment, the invention relates to a compound of formula (XII),

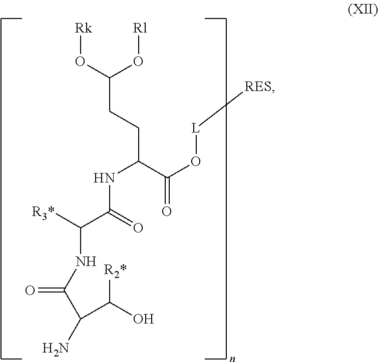

especially (XIIA)

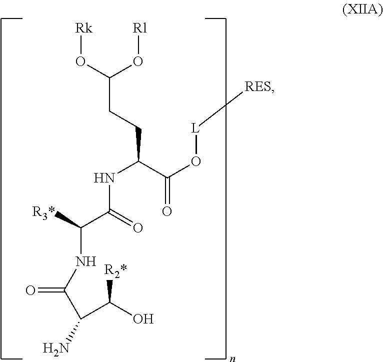

wherein Rk, Rl, R₂* and R₃* are as defined for a compound of formula (II) above,
L is a cleavable linker, RES is a solid resin and n is a natural number not including 0.

In a preferred embodiment, the invention relates to a compound of formula (XII), especially of the formula (XIIA), wherein
R₂* is methyl;
R₃* is iso-butyl, sec-butyl, or iso-propyl; and
Rk and Rl are each independently of each other C₁₋₈-alkyl or benzyl, or Rk and Rl together form a —CH₂—CH₂— or —CH₂—CH₂—CH₂— bridge.

In a more preferred embodiment, the invention relates to a compound of formula (XII), especially of the formula (XIIA), wherein

R₂* is methyl;
R₃* is iso-butyl;
Rk and Rl together form a —CH₂—CH₂— bridge;

### L is 2Cl-trityl; and

RES is divinylbenzene crosslinked polystyrene.

In another embodiment, the present invention relates to the use of the compound of formula (XII), or salt thereof, especially of the formula (XIIA), or a salt thereof, for the synthesis of a compound of formula (I), or salt thereof, especially of the formula (IA), or a salt thereof.

In a further embodiment, the invention relates to a compound of formula (XIV),

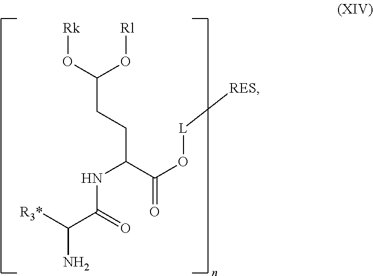

especially (XIVA)

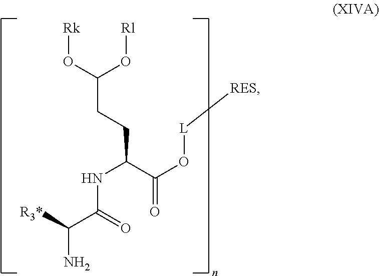

wherein Rk, Rl, and R₃* are as defined for a compound of formula (II) above,
L is a cleavable linker, RES is a solid resin and n is a natural number not including 0.

In a preferred embodiment, the invention relates to a compound of formula (XIV), especially of the formula (XIVA), wherein
R₃* is iso-butyl, sec-butyl, or iso-propyl; and
Rk and Rl are each independently of each other C₁₋₈-alkyl or benzyl, or Rk and Rl together form a —CH₂—CH₂— or —CH₂—CH₂—CH₂— bridge.

In a more preferred embodiment, the invention relates to a compound of formula (XIV), especially of the formula (XIVA), wherein

R₃* is iso-butyl;
Rk and Rl together form a —CH₂—CH₂— bridge;

### L is 2Cl-trityl; and

RES is divinylbenzene crosslinked polystyrene.

In another embodiment, the present invention relates to the use of the compound of formula (XIV), or salt thereof, especially of the formula (XIVA), or a salt thereof, for the synthesis of a compound of formula (I), or salt thereof, especially of the formula (IA), or a salt thereof.

In a further embodiment, the invention relates to a compound of formula (XVI),

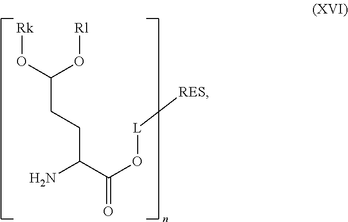

especially (XVIA)

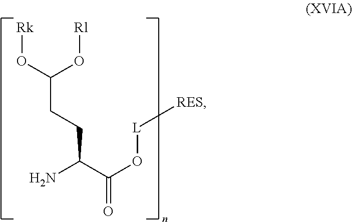

wherein Rk and Rl are as defined for a compound of formula (II) above,
L is a cleavable linker, RES is a solid resin and n is a natural number not including 0.

In a preferred embodiment, the invention relates to a compound of formula (XVI), especially of the formula (XVIA), wherein
Rk and Rl are each independently of each other C₁₋₈-alkyl or benzyl, or Rk and Rl together form a —CH₂—CH₂— or —CH₂—CH₂—CH₂— bridge.

In a more preferred embodiment, the invention relates to a compound of formula (XVI), especially of the formula (XVIA), wherein

Rk and Rl together form a —CH₂—CH₂— bridge;

### L is 2Cl-trityl; and

RES is divinylbenzene crosslinked polystyrene.

In another embodiment, the present invention relates to the use of the compound of formula (XVI), or salt thereof, especially of the formula (XVIA), or a salt thereof, for the synthesis of a compound of formula (I), or salt thereof, especially of the formula (IA), or a salt thereof.

Method II, Section A′: Conversion of a Compound of Formula (II′), or a Salt Thereof, into a Compound of Formula (I), or a Salt Thereof

In one embodiment, the invention relates to a process for the preparation of a cyclic depsipeptide compound of formula (I), or a salt thereof,

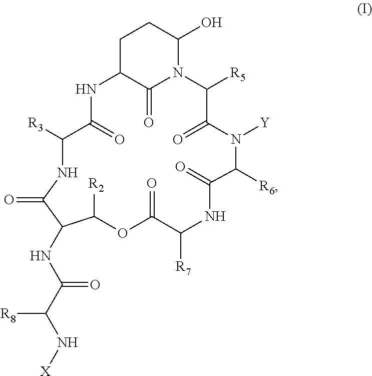

especially of the formula (IA), or a salt thereof,

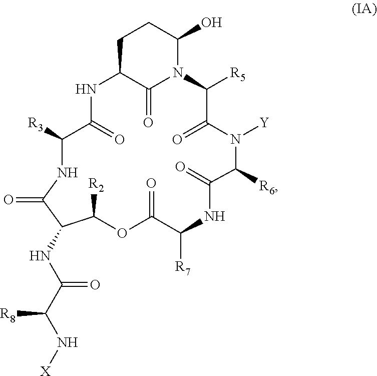

wherein
X is C₁₋₉-acyl;
R₂ is C₁₋₈-alky;
R₃ is the side chain of an alpha-amino acid;
R₅ is the side chain of an alpha-amino acid;
R₆ is the side chain of an alpha-amino acid, wherein the side chain contains a hydroxy group;
R₇ is the side chain of an alpha-amino acid;
R₈ is the side chain of an alpha-amino acid, wherein the side chain contains a terminal carboxy or carbamoyl group; and
Y is hydrogen or C₁₋₈-alkyl;
said process comprising
submitting compound of formula (II′), or a salt thereof,

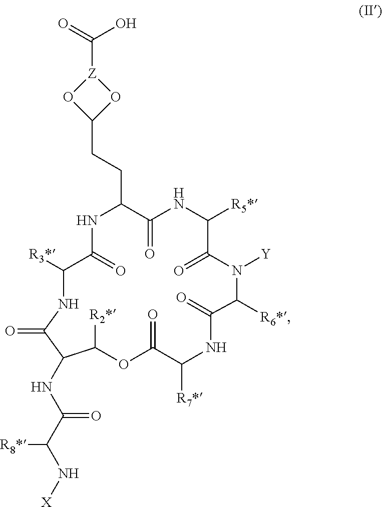

especially of the formula (II′A), or a salt thereof,

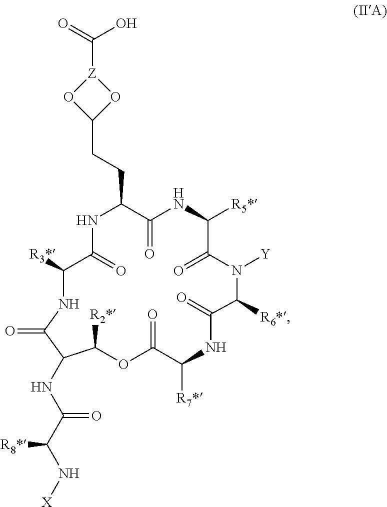

wherein Z is a linear or branched C₂₋₈-alkylene bridge, where Z together with the two oxygen atoms and the carbon atom to which the two oxygen atoms are bound, form a 5-7 membered ring, Y and X are as defined for a compound of formula (I) and R₂*′, R₃*′, R₅*′, R₆*′, R₇*′ and R₈*′ correspond to R₂, R₃, R₅, R₆, R₇ and R₈ in formula (I), respectively, but with the proviso that reactive functional groups on these residues are present in protected form, if they could participate in undesired side reactions, to acetal deprotecting conditions.

Typically, acetal deprotecting conditions in Section A′ comprise acid catalyzed transacetalization in acetone or hydrolysis in wet solvents or in aqueous acid, especially an alpha-halo substituted alkanoic acid, such as trifluoroacetic acid or trichloroacetic acid. For example, the reaction is typically carried out in a suitable solvent in the presence of a suitable acid at a temperature range between 0° C. and 40° C. Preferably the acid used is TFA, the solvent is DCM and the reaction is carried out at rt.

Depending on the choice of protecting groups (if present) on R₂*′, R₃*′, R₅*′, R₆*′, R₇*′ and R₈*′, Section A′ of Method II is a one step process, wherein all protecting groups present in a compound of formula (II′), or a salt thereof, especially (II′A), or a salt thereof, are be deprotected under acetal deprotecting conditions, or Section A′ of Method II is a multi-step process, comprising further steps for the deprotection of protecting groups (if present) on R₂*′, R₃*′, R₅*′, R₆*′, R₇*′ and R₈*′.

Preferably the Section A′ of Method II is a one step process, wherein protecting groups (if present) on R₂*′, R₃*′, R₅*′, R₆*′, R₇*′ and R₈*′ are chosen so that these protecting groups are deprotected when the compound of formula (II′), or a salt thereof, especially (II′A), or a salt thereof is submitted to acetal deprotection conditions.

Preferably, for any of the processes detailed in Section A′,

X is acetyl or isobutyryl;
R₂ and R₂*′ are methyl;
R₃ and R₃*′ are iso-butyl, sec-butyl, or iso-propyl;
R₅ and R₅*′ are benzyl, iso-butyl, sec-butyl, or iso-propyl;
R₆ is 4-hydroxybenzyl;
R₆*′ is 4-hydroxybenzyl, wherein the OH group is protected with a suitable protecting group;
R₇ and R₇*′ are iso-butyl, sec-butyl or iso-propyl;
R₈ is 2-amino-2-oxoethyl or 3-amino-3-oxopropyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge; and
Y is methyl.

More preferably, for any of the processes detailed in Section A′,

X is isobutyryl;
R₂ and R₂* are methyl;
R₃ and R₃* are iso-butyl;
R₅ and R₅* are sec-butyl;
R₆ is 4-hydroxybenzyl;
R₆* is 4-hydroxybenzyl, wherein the OH group is protected with a suitable protecting group;
R₇ and R₇* are sec-butyl;
R₈ is 3-amino-3-oxopropyl;
R₈* is 3-amino-3-oxopropyl wherein the NH₂ group of the carbamoyl is protected with a suitable protecting group;
Z is a —CH₂—CH₂— bridge; and
Y is methyl.
Method II, Section B′: Conversion of a Compound of Formula (III′) into a Compound of Formula (II′), or a Salt Thereof

In another embodiment of the invention relates to a process for the preparation of a compound formula (II′), or a salt thereof,

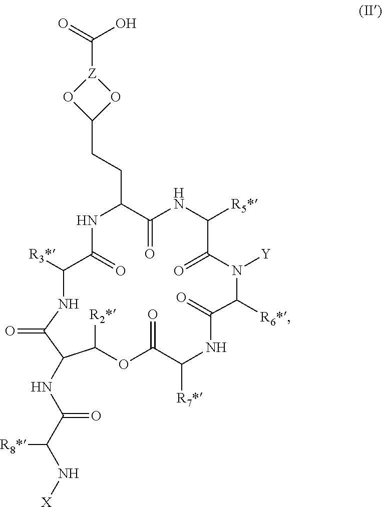

especially of the formula (II′A), or a salt thereof,

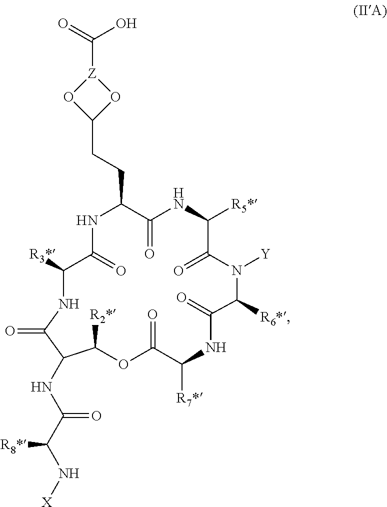

wherein Y and X are as defined for a compound of formula (I) above and Z, R₂*′, R₃*′, R₅*′, R₆*′, R₇*′ and R₈*′ are as defined for a compound of formula (II′) above,
said process comprising
submitting a compound of formula (III′),

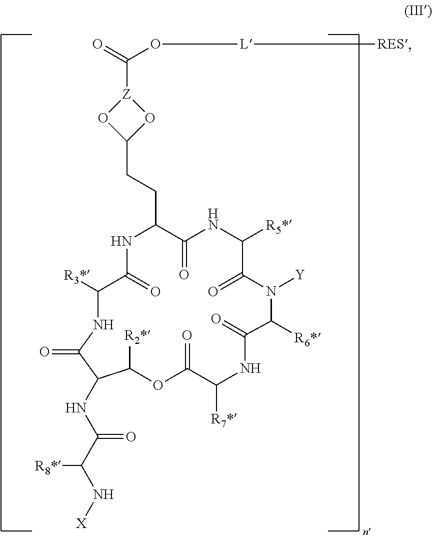

especially of the formula (III′A),

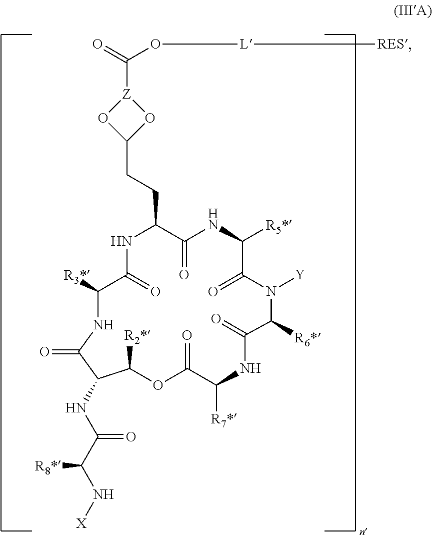

wherein Y and X are as defined for a compound of formula (I) above and Z, R₂*′, R₃*′, R₅*′, R₆*′, R₇*′ and R₈*′ are as defined for a compound of formula (II′) above,
L′ is a cleavable linker, RES′ is a solid resin and n′ is a natural number not including 0, to cleavage conditions.

Typically, cleavage conditions in Section B′ are conditions that detach a compound from solid support (RES′-L′-) used for SPPS. These conditions depend on the nature of the solid support, mainly on the nature of the linker L′.

Typically, cleavage conditions in Section B′ are very mild acidic conditions for example treatment with AcOH/TFE/DCM or with HFIP in an appropriate solvent, e.g. in dichloromethane or trifluoroethanol. Preferably the cleavage reagent is HFIP, the solvent is DCM and the reaction is carried out at rt.

Preferably the cleavage conditions are chosen so that the protecting groups (if present) on R₂*′, R₃*′, R₅*′, R₆*′, R₇*′ and R₈*′ are conserved.

Preferably, for any of the processes detailed in Section B′,

X is acetyl or isobutyryl;
R₂*′ is methyl;
R₃*′ is iso-butyl, sec-butyl, or iso-propyl;
R₅*′ is benzyl, iso-butyl, sec-butyl, or iso-propyl;
R₆*′ is 4-hydroxybenzyl, wherein the OH group is protected with a suitable protecting group;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge; and
Y is methyl.

More preferably, for any of the processes detailed in Section B′,

X is isobutyryl;
R₂*′ is methyl;
R₃*′ is iso-butyl;
R₅*′ is sec-butyl;
R₆*′ is 4-hydroxybenzyl, wherein the OH group is protected with a suitable protecting group;
R₇*′ is sec-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene; and
Y is methyl.
Method II, Section C′: Conversion of a Compound of Formula (IV′) into a Compound of Formula (III′)

In another embodiment of the invention relates to a process for the preparation of a compound formula (III′)

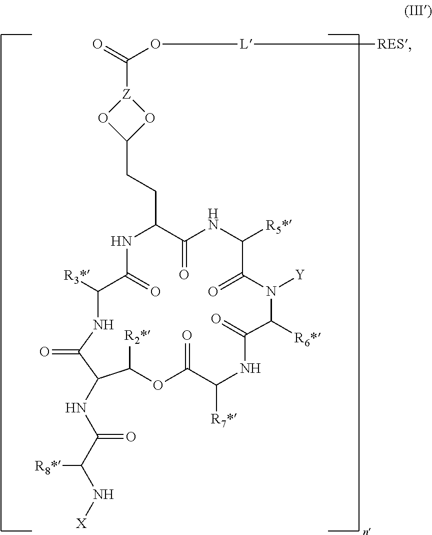

especially of the formula (III′A),

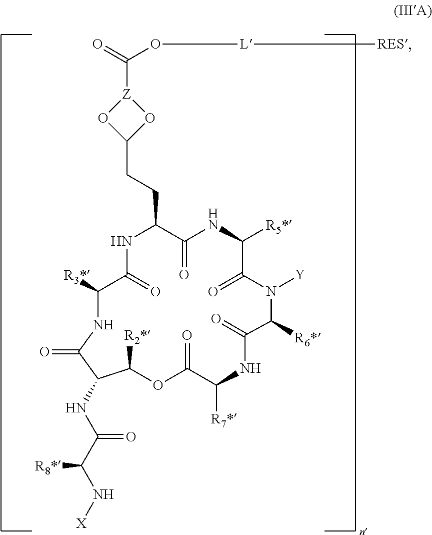

wherein Y and X are as defined for a compound of formula (I) above and Z, R₂*′, R₃*′, R₅*′, R₆*′, R₇*′ and R₈*′ are as defined for a compound of formula (II′) above,
L′ is a cleavable linker, RES′ is a solid resin and n′ is a natural number not including 0, said process comprising
submitting a compound of formula (IV′),

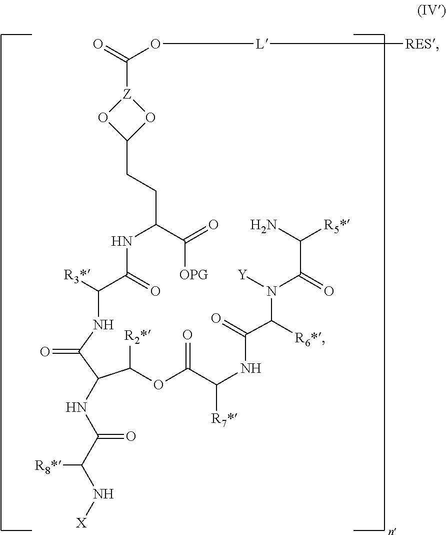

especially (IV′A),

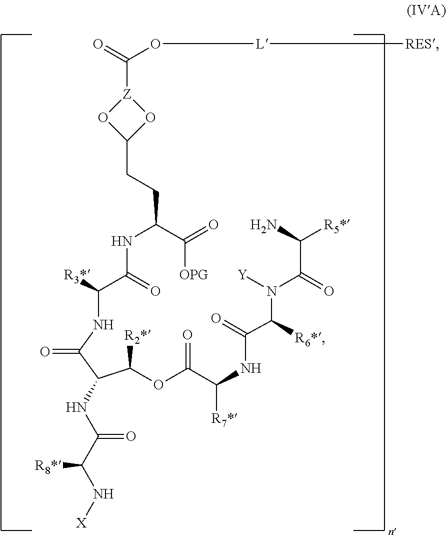

wherein Y and X are as defined for a compound of formula (I) above and Z, R₂*′, R₃*′, R₅*′, R₆*′, R₇*′ and R₈*′ are as defined for a compound of formula (II′) above,
L′ is a cleavable linker, RES′ is a solid resin, n′ is a natural number not including 0 and PG is a carboxy protecting group,
to macrolactamization conditions.

Typically, macrolactamization conditions in Section C′ are conditions for the coupling of a carboxy group to an amine group. The reaction is typically carried out using activating conditions for the activation of the carboxy group. Preferably, macrolactamization conditions use a coupling agents in a suitable at a temperature range between 0° C. and 40° C. Preferably the macrolactamization conditions use Oxyma/DICI, the solvent is DMF and the reaction is carried out at rt.

Preferably, for any of the processes detailed in Section C′,

X is acetyl or isobutyryl;
R₂*′ is methyl;
R₃*′ is iso-butyl, sec-butyl, or iso-propyl;
R₅*′ is benzyl, iso-butyl, sec-butyl, or iso-propyl;
R₆*′ is 4-hydroxybenzyl, wherein the OH group is protected with a suitable protecting group;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge;
PG is allyl, 2-methyl-2-propenyl, 3-methylbut-2-enyl or 2-methylbut-3-en-2-yl; and
Y is methyl.

More preferably, for any of the processes detailed in Section C′,

X is isobutyryl;
R₂*′ is methyl;
R₃*′ is iso-butyl;
R₅*′ is sec-butyl;
R₆*′ is 4-hydroxybenzyl, wherein the OH group is protected with a suitable protecting group;
R₇*′ is sec-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene;
PG is allyl and
Y is methyl.
Method II, Section D′: Conversion of a Compound of Formula (XVI′), into a Compound of Formula (IV′)

In another embodiment of the invention relates to a process for the preparation of a compound formula (IV′), said process comprising submitting a compound of formula (XVI′) to Solid Phase Peptide Synthesis (SPPS). This process has several cycles.

Method II, Section D′, Cycle 7′: Conversion of a Compound of Formula (VI′), into a Compound of Formula (IV′)

In another embodiment of the invention relates to a process for the preparation of a compound formula (IV′)

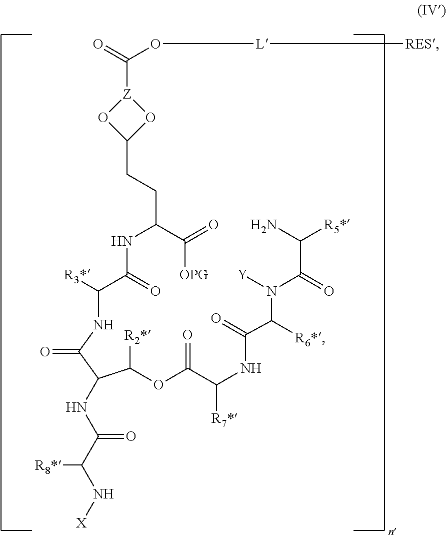

especially (IV′A),

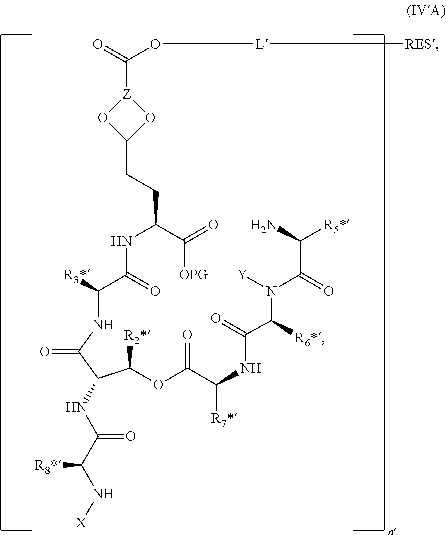

wherein Y and X are as defined for a compound of formula (I) above and Z, R₂*′, R₃*′, R₅*′, R₆*′, R₇*′ and R₈*′ are as defined for a compound of formula (II′) above,
L′ is a cleavable linker, RES′ is a solid resin, n′ is a natural number not including 0 and PG is a carboxy protecting group,
said process comprising
step 1′ of Method II, Section D′, cycle 7′ of
reacting a compound of formula (VI′),

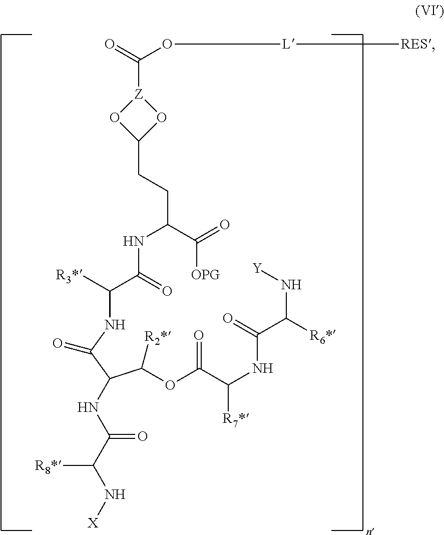

especially (VI′A),

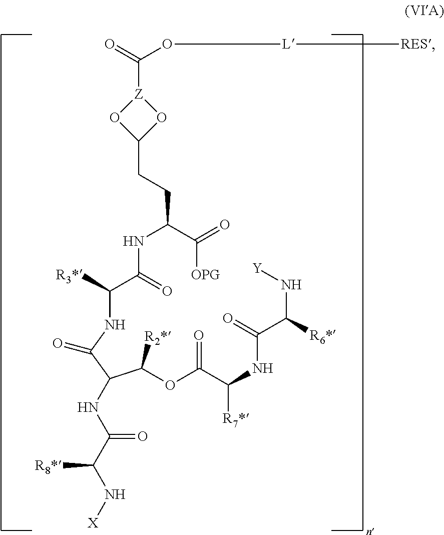

wherein Y and X are as defined for a compound of formula (I) above and Z, R₂*′, R₃*′, R₆*′, R₇*′ and R₈*′ are as defined for a compound of formula (II′) above,
L′ is a cleavable linker, RES′ is a solid resin, n′ is a natural number not including 0 and PG is a carboxy protecting group,
with a compound of formula (V′)

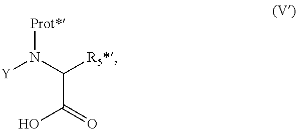

especially (V′A),

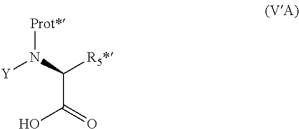

wherein R₅*′ is as defined for a compound of formula (II′) above and Prot*′ is an amino protecting group,
under peptide coupling conditions;
followed by step 2′ of Method II, Section D′, cycle 7′ of
removing the protecting group Prot*′
under conditions for removal of an amine protecting group.
Method II, Section D′, Cycle 6′: Conversion of a Compound of Formula (VIII′), into a Compound of Formula (VI′)

In another embodiment of the invention relates to a process for the preparation of a compound formula (VI′)

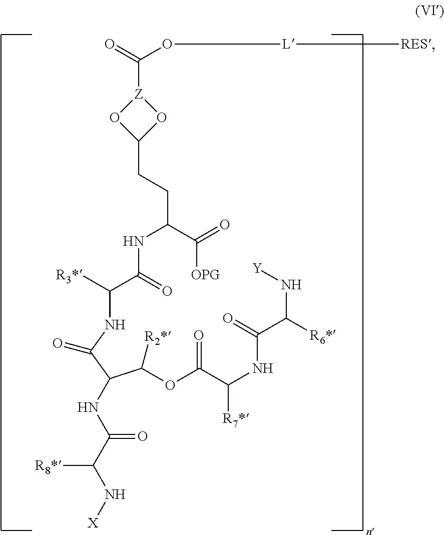

especially (VI′A),

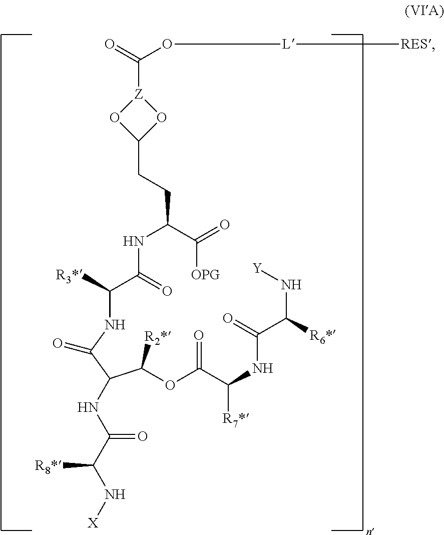

wherein Y and X are as defined for a compound of formula (I) above and Z, R₂*′, R₃*′, R₆*′, R₇*′ and R₈*′ are as defined for a compound of formula (II′) above,
L′ is a cleavable linker, RES′ is a solid resin, n′ is a natural number not including 0 and PG is a carboxy protecting group,
said process comprising
step 1′ of Method II, Section D′, cycle 6′ of
reacting a compound of formula (VIII′),

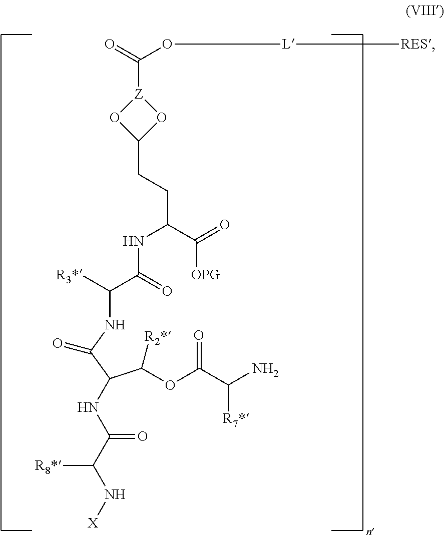

especially (VIII′A),

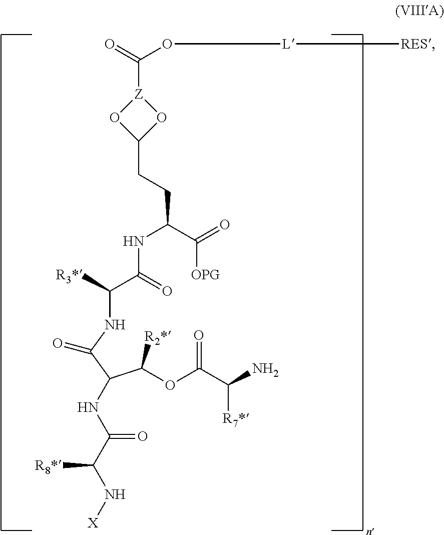

wherein Y and X are as defined for a compound of formula (I) above and Z, R₂*′, R₃*′, R₇*′ and R₈*′ are as defined for a compound of formula (II′) above,
L′ is a cleavable linker, RES′ is a solid resin, n′ is a natural number not including 0 and PG is a carboxy protecting group,
with a compound of formula (VII′)

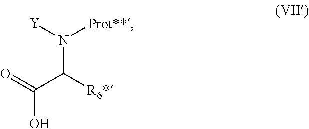

especially (VII′A),

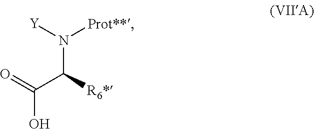

wherein Y is as defined for a compound of formula (I) above, R₆*′ is as defined for a compound of formula (II′) above and Prot**′ is an amino protecting group
under peptide coupling conditions;
followed by step 2′ of Method II, Section D′, cycle 6′ of
removing the protecting group Prot**′
under conditions for removal of an amine protecting group.
Method II, Section D′, Cycle 5′: Conversion of a Compound of Formula (X′), into a Compound of Formula (VIII′)

In another embodiment of the invention relates to a process for the preparation of a compound formula (VIII′),

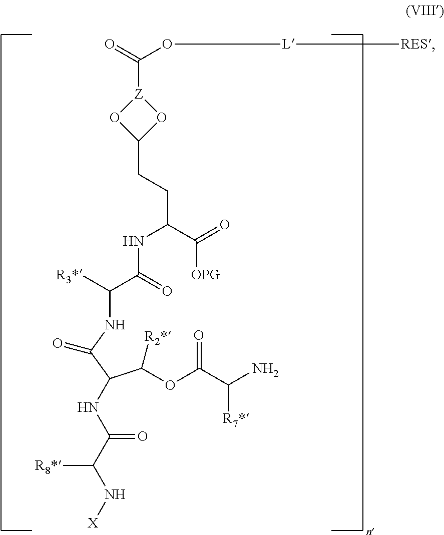

especially (VIII′A),

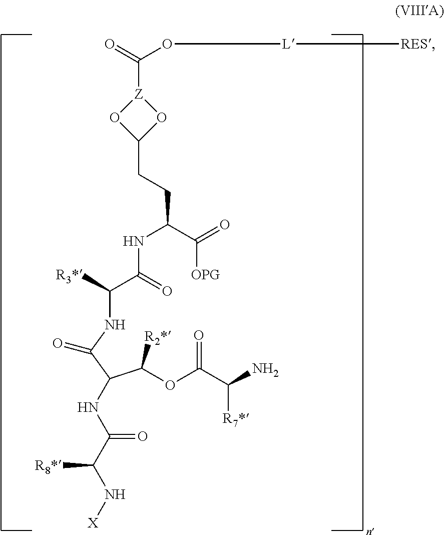

wherein Y and X are as defined for a compound of formula (I) above and Z, R₂*′, R₃*′, R₇*′ and R₈*′ are as defined for a compound of formula (II′) above,
L′ is a cleavable linker, RES′ is a solid resin, n′ is a natural number not including 0 and PG is a carboxy protecting group,
said process comprising
step 1′ of Method II, Section D′, cycle 5′ of
reacting a compound of formula (X′),

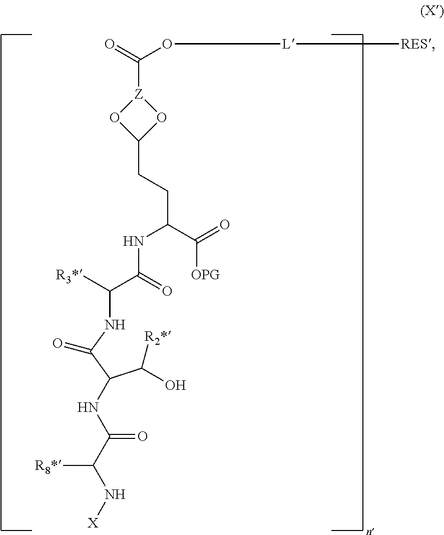

especially (X′A),

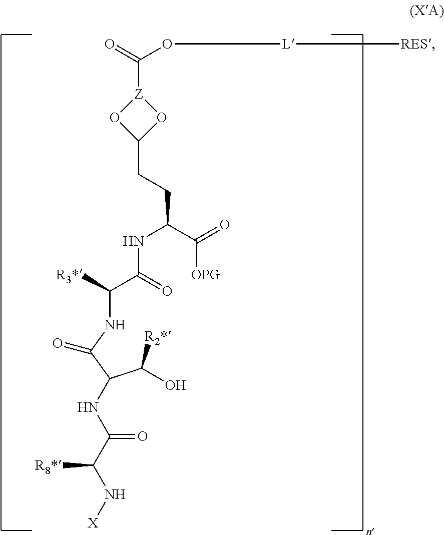

wherein X is as defined for a compound of formula (I) above and Z, R₂*′, R₃*′ and R₈*′ are as defined for a compound of formula (II′) above,
L′ is a cleavable linker, RES′ is a solid resin, n′ is a natural number not including 0 and PG is a carboxy protecting group,
with a compound of formula (IX′)

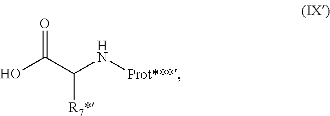

especially (IX′A)

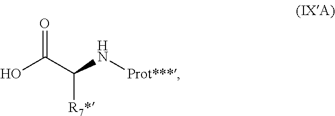

wherein R₇*′ is as defined for a compound of formula (II′) above and Prot***′ is an amino protecting group
under ester coupling conditions;
followed by step 2′ of Method II, Section D′, cycle 5′ of
removing the protecting group Prot***′
under conditions for removal of an amine protecting group.

Typically, for the ester coupling conditions mentioned in Section D′ cycle 5′, conditions similar to peptide coupling conditions are used. Preferable ester coupling conditions use MSNT (1-(mesitylene-2-sulfonyl)-3-nitro-1,2,4-triazole) in the presence of NMI (N-methylimidazole) in DMF and the reaction is carried out at rt.

Method II, Section D′, Cycle 4′: Conversion of a Compound of Formula (XI′), into a Compound of Formula (X′)

In another embodiment of the invention relates to a process for the preparation of a compound formula (X′),

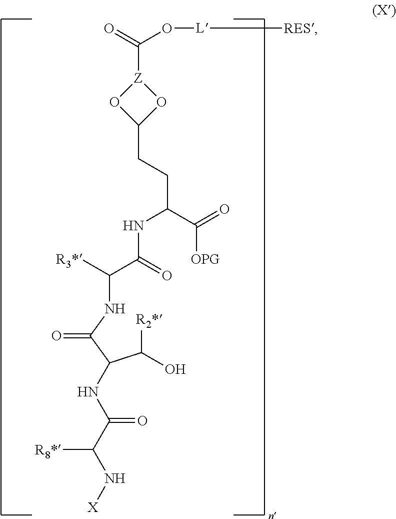

especially (X′A),

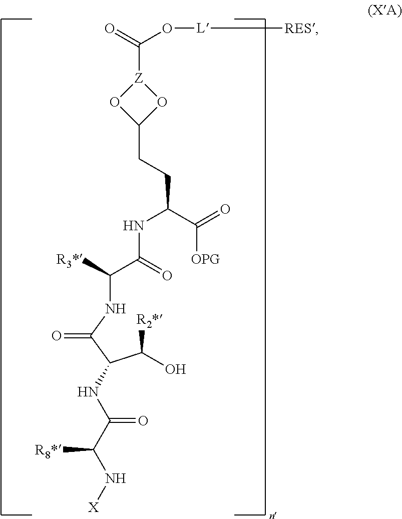

wherein X is as defined for a compound of formula (I) above and Z, R₂*′, R₃*′ and R₈*′ are as defined for a compound of formula (II′) above,
L′ is a cleavable linker, RES′ is a solid resin, n′ is a natural number not including 0 and PG is a carboxy protecting group,
said process comprising
step 1′ of Method II, Section D′, cycle 4′ of
reacting a compound of formula (XI′),

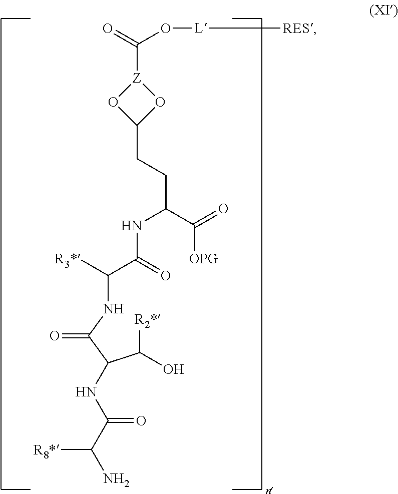

especially (XI′A),

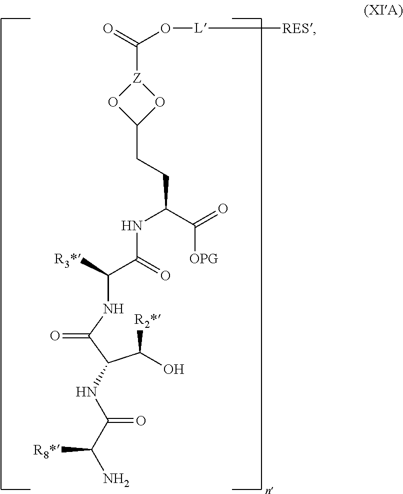

wherein Z, R₂*′, R₃*′ and R₈*′ are as defined for a compound of formula (II′) above,
L′ is a cleavable linker, RES′ is a solid resin, n′ is a natural number not including 0 and PG is a carboxy protecting group,
with a X—OH
wherein X is as defined for a compound of formula (I) above
under peptide coupling conditions.
Method II, Section D′, Cycle 3′: Conversion of a Compound of Formula (XII′), into a Compound of Formula (XI′)

In another embodiment of the invention relates to a process for the preparation of a compound formula (XI′),

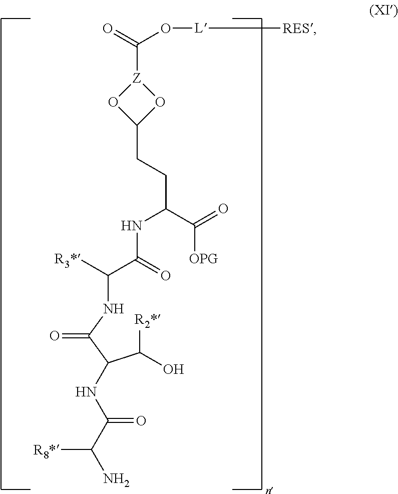

especially (XI′A),

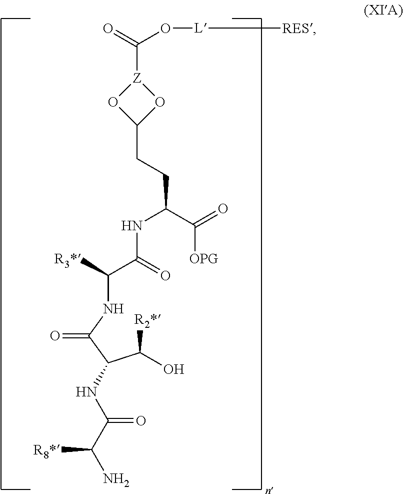

wherein Z, R₂*′, R₃*′ and R₈*′ are as defined for a compound of formula (II′) above,
L′ is a cleavable linker, RES′ is a solid resin, n′ is a natural number not including 0 and PG is a carboxy protecting group,
said process comprising
step 1 of Method II, Section D′, cycle 3′ of
reacting a compound of formula (XII′),

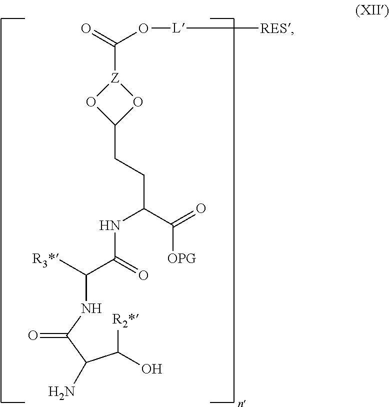

especially (XII′A)

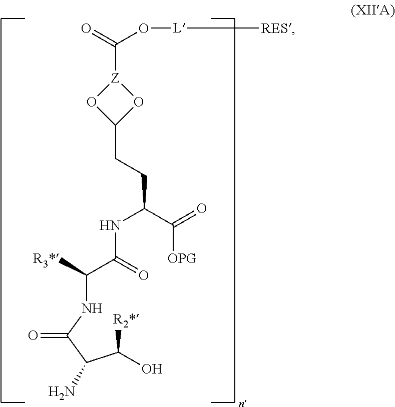

wherein Z, R₂*′ and R₃*′ are as defined for a compound of formula (II′) above,
L′ is a cleavable linker, RES′ is a solid resin, n′ is a natural number not including 0 and PG is a carboxy protecting group,
with a compound of formula (XIII′)

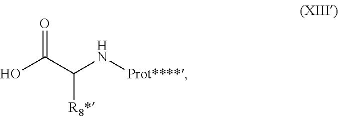

especially (XIII′A)

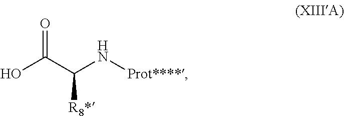

wherein R₈*′ is as defined for a compound of formula (II′) above and Prot****′ is an amino protecting group,
under peptide coupling conditions;
followed by step 2′ of Method II, Section D′, cycle 3′ of
removing the protecting group Prot****′
under conditions for removal of an amine protecting group.
Method II, Section D′, Cycle 2′: Conversion of a Compound of Formula (XIV′), into a Compound of Formula (XII′)

In another embodiment of the invention relates to a process for the preparation of a compound formula (XII′),

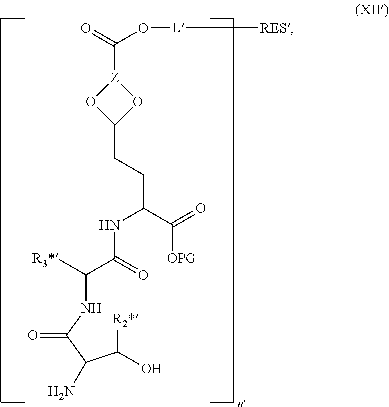

especially (XII′A)

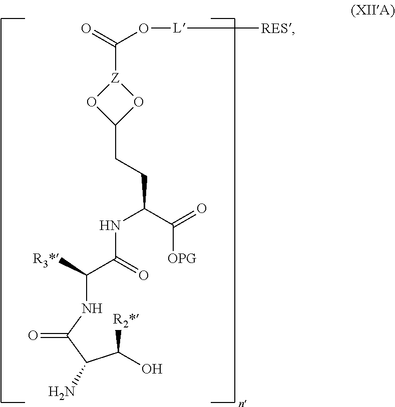

wherein Z, R₂*′ and R₃*′ are as defined for a compound of formula (II′) above,
L′ is a cleavable linker, RES′ is a solid resin, n′ is a natural number not including 0 and PG is a carboxy protecting group,
said process comprising
step 1′ of Method II, Section D′, cycle 2′ of
reacting a compound of formula (XIV′),

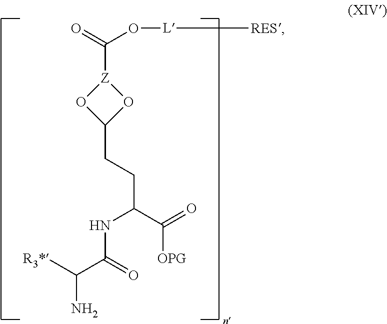

especially (XIV′A)

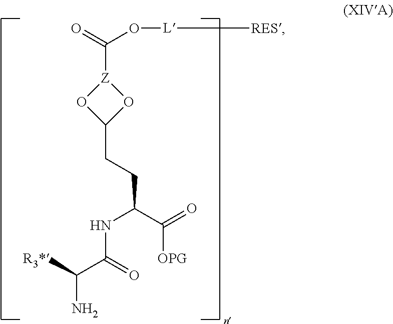

wherein Z and R₃*′ are as defined for a compound of formula (II′) above,
L′ is a cleavable linker, RES′ is a solid resin, n′ is a natural number not including 0 and PG is a carboxy protecting group,
with a compound of formula (XV′)

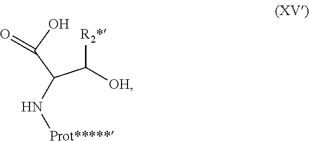

especially (XV′A)

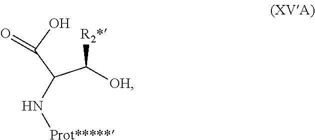

wherein R₂*′ is as defined for a compound of formula (II′) above and Prot*****′ is an amino protecting group,
under peptide coupling conditions;
followed by step 2′ of Method II, Section D′, cycle 2′ of
removing the protecting group Prot*****′
under conditions for removal of an amine protecting group.
Method II, Section D′, Cycle 1′: Conversion of a Compound of Formula (XVI′), into a Compound of Formula (XIV′)

In another embodiment of the invention relates to a process for the preparation of a compound formula (XIV′),

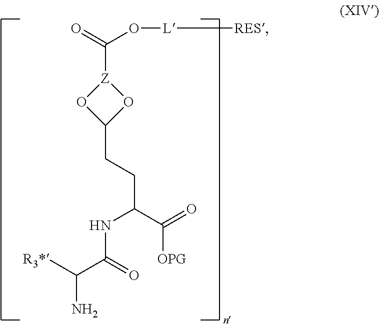

especially (XIV′A)

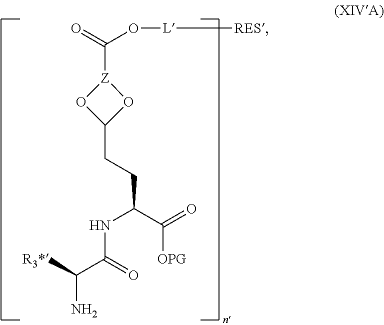

wherein Z and R₃*′ are as defined for a compound of formula (II′) above,
L′ is a cleavable linker, RES′ is a solid resin, n′ is a natural number not including 0 and PG is a carboxy protecting group,
said process comprising
step 1′ of Method II, Section D′, cycle 1′ of
reacting a compound of formula (XVI′),

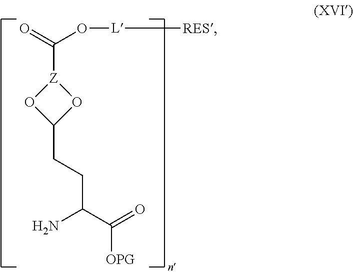

especially (XVI′A)

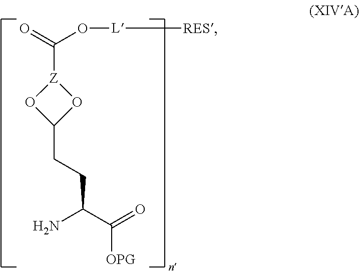

wherein Z is as defined for a compound of formula (II′) above,
L′ is a cleavable linker, RES′ is a solid resin, n′ is a natural number not including 0 and PG is a carboxy protecting group,
with a compound of formula (XVII′)

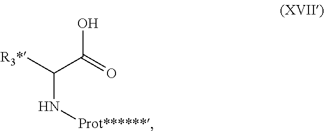

especially (XVI I′A)

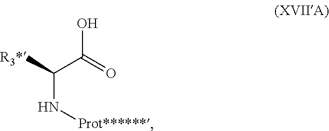

wherein R₃*′ is as defined for a compound of formula (II′) above and Prot******′ is an amino protecting group,
under peptide coupling conditions;
followed by step 2′ of Method II, Section D′, cycle 1′ of
removing the protecting group Prot******′
under conditions for removal of an amine protecting group.

Typically, peptide coupling conditions mentioned in Section D′ are carried out using a coupling agent preferably in the presence of a mild base, typically in the presence of an appropriate solvent or solvent mixture, e.g. an N,N dialkylformamide, such as dimethylformamide, a halogenated hydrocarbon, e.g. dichloromethane, N-alkylpyrro-lidones, such as N-methylpyrrolidone, nitriles, e.g. acetonitrile, ethers, such as dioxane or tetrahydrofurane, or aromatic hydrocarbons, e.g. toluene, or mixtures of two or more, where, provided an excess of coupling agent is present, also water may be present. The temperatures may be ambient temperature or lower or higher, e.g. in the range from −20° C. to 50° C. Preferable peptide coupling conditions use HATU (2-(1H-7-azabenzotriazol-1-yl)-1,1,3,3-tetramethyluronium hexafluorophosphate methanaminium) in the presence of DIEPA (N,N-diisopropylethylamine) or Oxyma (ethyl 2-cyano-2-(hydroxyimino)acetate)/DICI (diisopropylcarbodiimide), in DMF and the reaction is carried out at rt.

Typically, conditions for removal of an amine protecting group in Section D′ are basic conditions. The reaction is typically carried out in a suitable solvent, e.g. an N,N dialkylformamide, such as dimethylformamide, a halogenated hydrocarbon, e.g. dichloromethane, alkanols, such as ethanol, propanol or isopropanol, nitriles, e.g. acetonitrile, or aromatic hydrocarbons, e.g. toluene, or mixtures of two or more, also water may be present, in the presence of a suitable mild base, such as piperidine, morpholine, dicyclohexylamine, p-dimethylamino-pyridine, diisopropylamine, piperazine, tris-(2-aminoethyl)amine in an appropriate solvent, e.g. N,N-dimethylformamide, methylene chloride, at a temperature range between 0° C. and 40° C. Preferably the base used is piperidine, the solvent is DMF and the reaction is carried out at rt.

Preferably the peptide coupling conditions and conditions for removal of an amine protecting group in Section D′ are chosen so that the protecting groups (if present) on R₂*, R₃*, R₅*, R₆*, R₇* and R₈* (where present) and the groups Rk and Rl are conserved and ester groups (where present) are not hydrolyzed.

Preferably, for any of the processes detailed in Section D′,

X (where present) is acetyl or isobutyryl;
R₂*′ (where present) is methyl;
R₃*′ (where present) is iso-butyl, sec-butyl, or iso-propyl;
R₅*′ (where present) is benzyl, iso-butyl, sec-butyl, or iso-propyl;
R₆*′ (where present) is 4-hydroxybenzyl, wherein the OH group is protected with a suitable protecting group;
R₇*′ (where present) is iso-butyl, sec-butyl or iso-propyl;
R₈*′ (where present) is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge;
Y (where present) is methyl;
PG is allyl, 2-methyl-2-propenyl, 3-methylbut-2-enyl or 2-methylbut-3-en-2-yl; and
Prot*′, Prot**′, Prot***′, Prot****′, Prot*****′ and Prot******′ (where present) are selected from fluoren-9-ylmethoxycarbonyl (Fmoc); 2-(2′ or 4′-pyridyl)ethoxycarbonyl and 2,2-bis(4′nitrophenyl)ethoxycarbonyl.

More preferably, for any of the processes detailed in Section D′,

X (where present) is isobutyryl;
R₂*′ (where present) is methyl;
R₃*′ (where present) is iso-butyl;
R₅*′ (where present) is sec-butyl;
R₆*′ (where present) is 4-hydroxybenzyl, wherein the OH group is protected with a suitable protecting group;
R₇*′ (where present) is sec-butyl;
R₈*′ (where present) is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene; and
Y (where present) is methyl;
PG is allyl; and
Prot*′, Prot**′, Prot***′, Prot****′, Prot*****′ and Prot******′ (where present) are Fmoc.
Method II, Section E′: Conversion of a Compound of Formula (XVII′), or a Salt Thereof, into a Compound of Formula (XVI′)

In another embodiment of the invention relates to a process for the preparation of a compound formula (XVI′)

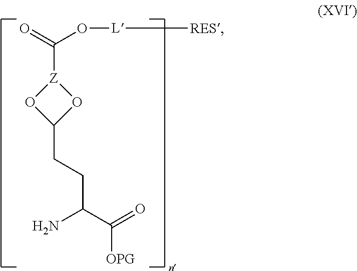

especially (XVI′A)

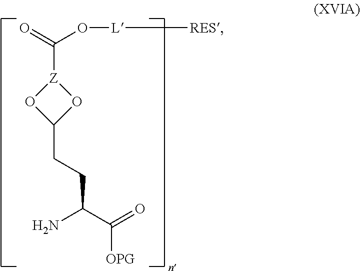

wherein Z is as defined for a compound of formula (II′) above,
L′ is a cleavable linker, RES′ is a solid resin, n′ is a natural number not including 0 and PG is a carboxy protecting group,
said process comprising
step 1′ of Method II, Section E′, of
submitting a compound of formula (XVII′),

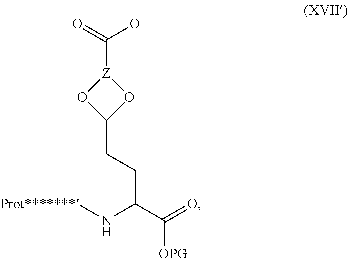

especially (XVII′A)

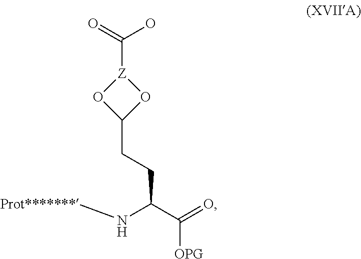

wherein Z is as defined for a compound of formula (II′) above, PG is a carboxy protecting group and Prot*******′ is an amino protecting group,
to conditions for loading to solid support;
followed by step 2′ of Method II, Section E′, of
removing the protecting group Prot*******′
under conditions for removal of an amine protecting group.

Typically, conditions for loading to solid support (RES′-L′-) in Section E′ depend on the nature of the solid support, mainly on the nature of the linker L′. For example the unloaded solid support is suspended in an appropriate solvent, such as a dialkyl acid amide, e.g. dimethylformamide, an alcohol, such as ethanol, propanol or isopropanol or dichloromethane and reacted with the carboxyl group of the compound to be loaded to solid support. Preferably, for the loading to chloro-(2′chloro)trityl-polystyrene resin (RES′-L′-CI, wherein RES′ is divinylbenzene crosslinked polystyrene and L′ is 2Cl-trityl), the resin is suspended in an appropriate solvent, e.g. dichloromethane and reacted with the carboxyl group containing compound to be loaded in the presence of a base, e.g. a tertiary amino base, such as DIPEA.

Typically, conditions for removal of an amine protecting group in Section E′ are mild basic conditions. The reaction is typically carried out in a suitable solvent, such as DMF or DCM in the presence of a suitable mild base, such as piperidine, morpholine, dicyclohexylamine, p-dimethylamino-pyridine, diisopropylamine, piperazine, tris-(2-aminoethyl)amine or 4-methylpiperidine at a temperature range between 0° C. and 40° C. Preferably the base used is piperidine, the solvent is DMF and the reaction is carried out at rt.

Preferably, for any of the processes detailed in Section E′,

Z is a —CH₂—CH₂— or —CH₂—CH₂—CH₂— bridge;
PG is allyl, 2-methyl-2-propenyl, 3-methylbut-2-enyl or 2-methylbut-3-en-2-yl; and
Prot*******′ is selected from fluoren-9-ylmethoxycarbonyl (Fmoc); 2-(2′ or 4′-pyridyl)ethoxycarbonyl and 2,2-bis(4′nitrophenyl)ethoxycarbonyl.

More preferably, for any of the processes detailed in Section E′,

Z is a —CH₂—CH₂— bridge;

### L′ is 2Cl-trityl;

RES′ is divinylbenzene crosslinked polystyrene;
PG is allyl; and

### Prot*******′ is Fmoc.

Method II, Section F′: Conversion of a Compound of Formula (XVIII′), or a Salt Thereof, into a Compound of Formula (XVII′)

In another embodiment of the invention relates to a process for the preparation of a compound formula (XVII′)

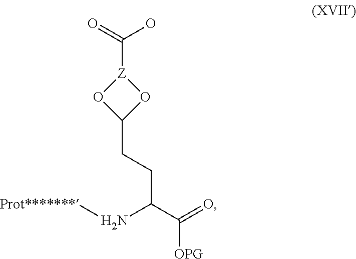

especially (XVI I′A)

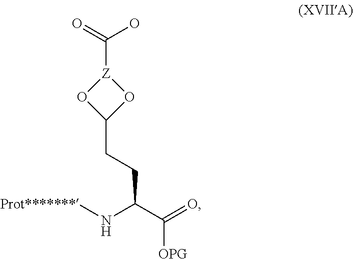

wherein Z is as defined for a compound of formula (II′) above, PG is a carboxy protecting group and Prot*******′ is an amino protecting group,
said process comprising
step 1′ of Method II, Section F′, of
reacting a compound of formula (XVIII′),

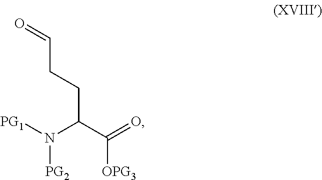

especially (XVIII′A)

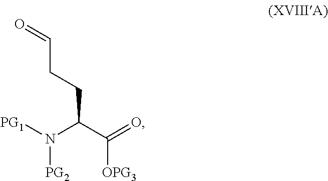

wherein PG₁ and PG₂ are amine protecting groups, and PG₃ is a carboxy protecting group,
with a substituted diol of the formula (XIX′),

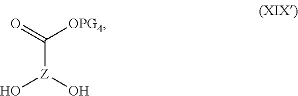

wherein PG₃ is a carboxy protecting group,
under acetal formation conditions;
followed by step 2′ of Method II, Section E′, of
removing the protecting groups PG₁ and PG₂
under conditions for removal of an amine protecting group;
followed by step 3′ of Method II, Section E′, of
re-protecting the free amine with an amino protecting group Prot*******′
under conditions for protecting an amine group;
followed by step 4′ of Method II, Section E′, of
removing the protecting group PG₃
under conditions for removal of a carboxy protecting group;
followed by step 5′ of Method II, Section E′, of
re-protecting the free carboxy group with a carboxy protecting group PG.
under conditions for protecting a carboxy group;
followed by step 6′ of Method II, Section E′, of
removing the protecting group PG₄
under conditions for removal of an carboxy protecting group.

Typically, acetal formation conditions in Section F′ comprise the presence of a Brönsted or a Lewis acid catalyst in a suitable solvent under water removal. A standard procedure for acetal formation employs for example toluenesulfonic acid as catalyst in refluxing toluene or benzene, under continuous removal of water; a mixture of orthoesters or molecular sieves can also provide effective water. Preferably the base used is piperidine, the solvent is DMF and the reaction is carried out at rt.

Typically, conditions for removal of an amine protecting group in Section F′ are mild basic conditions. The reaction is typically carried out in a suitable solvent, e.g. an N,N dialkylformamide, such as dimethylformamide, a halogenated hydrocarbon, e.g. dichloromethane, alkanols, such as ethanol, propanol or isopropanol, nitriles, e.g. acetonitrile, or aromatic hydrocarbons, e.g. toluene, or mixtures of two or more, also water may be present, in the presence of a suitable mild base, such as piperidine, morpholine, dicyclohexylamine, p-dimethylamino-pyridine, diisopropylamine, piperazine, tris-(2-aminoethyl)amine in an appropriate solvent, e.g. N,N-dimethylformamide, methylene chloride, at a temperature range between 0° C. and 40° C. Preferably the base used is piperidine, the solvent is DMF and the reaction is carried out at rt.

Typically, conditions for protecting an amine group in Section F′ are dependent on the choice of the amine protecting group. Such conditions are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

Typically, conditions for removal of a carboxy protecting group in Section F′ are dependent on the choice of the carboxy protecting group. Such conditions are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

Typically, conditions for protecting a carboxy group in Section F′ are ′ are dependent on the choice of the carboxy protecting group. Such conditions are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

Preferably, for any of the processes detailed in Section F′,

Z is a —CH₂—CH₂— or —CH₂—CH₂—CH₂— bridge;
PG is allyl, 2-methyl-2-propenyl, 3-methylbut-2-enyl or 2-methylbut-3-en-2-yl;
PG₁ is benzyl;
PG₂ is benzyl;
PG₃ is benzyl;
PG₄ is C₁₋₈-alkyl; and
Prot*******′ is selected from fluoren-9-ylmethoxycarbonyl (Fmoc); 2-(2′ or 4′-pyridyl)ethoxycarbonyl and 2,2-bis(4′nitrophenyl)ethoxycarbonyl.

More preferably, for any of the processes detailed in Section F′,

Z is a —CH₂—CH₂— bridge;
PG is allyl;
PG₁ is benzyl;
PG₂ is benzyl;
PG₃ is benzyl;
PG₄ is methyl; and

### Prot*******′ is Fmoc.

In another embodiment, the present invention relates to process for the preparation of a cyclic depsipeptide compound of formula (I), or a salt thereof, especially of the formula (IA), or a salt thereof, comprising
Method II, Section A′ as described herein;
Method II, Section B′ as described herein;
Method II, Section C′ as described herein;
Method II, Section D′ as described herein;
Method II, Section E′ as described herein; and
Method II, Section F′ as described herein.

In another embodiment, the present invention relates to process for the preparation of a cyclic depsipeptide compound of formula (I), or a salt thereof, especially of the formula (IA), or a salt thereof, comprising
Method II, Section A′ as described herein;
Method II, Section B′ as described herein;
Method II, Section C′ as described herein;
Method II, Section D′ as described herein; and
Method II, Section E′ as described herein.

In another embodiment, the present invention relates to process for the preparation of a cyclic depsipeptide compound of formula (I), or a salt thereof, especially of the formula (IA), or a salt thereof, comprising
Method II, Section A′ as described herein;
Method II, Section B′ as described herein;
Method II, Section C′ as described herein; and
Method II, Section D′ as described herein.

In another embodiment, the present invention relates to process for the preparation of a cyclic depsipeptide compound of formula (I), or a salt thereof, especially of the formula (IA), or a salt thereof, comprising
Method II, Section A′ as described herein;
Method II, Section B′ as described herein; and
Method II, Section C′ as described herein.

In another embodiment, the present invention relates to process for the preparation of a cyclic depsipeptide compound of formula (I), or a salt thereof, especially of the formula (IA), or a salt thereof, comprising
Method II, Section A′ as described herein; and
Method II, Section B′ as described herein.

In another embodiment, the present invention relates to process for the preparation of a compound of formula (II′), or a salt thereof, especially of the formula (II′A), or a salt thereof, comprising
Method II, Section B′ as described herein;
Method II, Section C′ as described herein;
Method II, Section D′ as described herein;
Method II, Section E′ as described herein; and
Method II, Section F′ as described herein.

In another embodiment, the present invention relates to process for the preparation of a compound of formula (II′), or a salt thereof, especially of the formula (II′A), or a salt thereof, comprising
Method II, Section B′ as described herein;
Method II, Section C′ as described herein;
Method II, Section D′ as described herein; and
Method II, Section E′ as described herein.

In another embodiment, the present invention relates to process for the preparation of a compound of formula (II′), or a salt thereof, especially of the formula (II′A), or a salt thereof, comprising
Method II, Section B′ as described herein;
Method II, Section C′ as described herein; and
Method II, Section D′ as described herein.

In another embodiment, the present invention relates to process for the preparation of a compound of formula (II′), or a salt thereof, especially of the formula (II′A), or a salt thereof, comprising
Method II, Section B′ as described herein; and
Method II, Section C′ as described herein.

In another embodiment, the present invention relates to process for the preparation of a compound of formula (III′), or a salt thereof, especially of the formula (III′A), or a salt thereof, comprising
Method II, Section C′ as described herein;
Method II, Section D′ as described herein;
Method II, Section E′ as described herein; and
Method II, Section F′ as described herein.

In another embodiment, the present invention relates to process for the preparation of a compound of formula (III′), or a salt thereof, especially of the formula (III′A), or a salt thereof, comprising
Method II, Section C′ as described herein;
Method II, Section D′ as described herein; and
Method II, Section E′ as described herein.

In another embodiment, the present invention relates to process for the preparation of a compound of formula (IV′), especially of the formula (IV′A), comprising
Method II, Section D′ as described herein;
Method II, Section E′ as described herein; and
Method II, Section F′ as described herein.

In another embodiment, the present invention relates to process for the preparation of a compound of formula (IV′), especially of the formula (IV′A), comprising
Method II, Section D′ as described herein; and
Method II, Section E′ as described herein.

In a further embodiment, the invention relates to a compound of formula (II′), or a salt thereof,

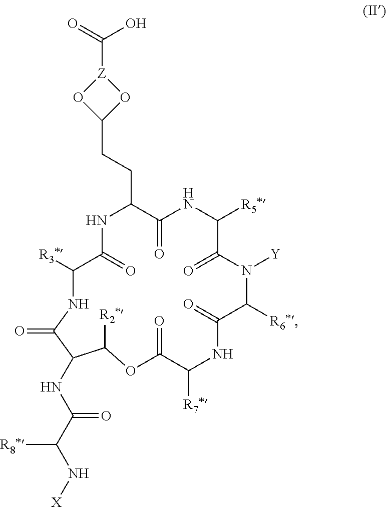

especially of the formula (II′A), or a salt thereof,

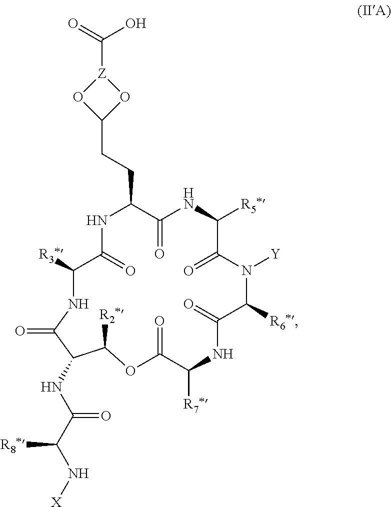

wherein Z is a linear or branched C₂₋₈-alkylene bridge, where Z together with the two oxygen atoms and the carbon atom to which the two oxygen atoms are bound, form a 5-7 membered ring, Y and X are as defined for a compound of formula (I) and R₂*′, R₃*′, R₅*′, R₆*′, R₇*′ and R₈*′ correspond to R₂, R₃, R₅, R₆, R₇ and R₈ in formula (I), respectively, but with the proviso that reactive functional groups on these residues are present in protected form, if they could participate in undesired side reactions

In a preferred embodiment, the invention relates to a compound of formula (II′), or a salt thereof, especially of the formula (II′A), or a salt thereof, wherein

X is acetyl or isobutyryl;
R₂*′ is methyl;
R₃*′ is iso-butyl, sec-butyl, or iso-propyl;
R₅*′ is benzyl, iso-butyl, sec-butyl, or iso-propyl;
R₆*′ is 4-hydroxybenzyl, wherein the OH group is protected with a suitable protecting group;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group; and
Y is methyl.

In a more preferred embodiment, the invention relates to a compound of formula (II′), or a salt thereof, especially of the formula (II′A), or a salt thereof, wherein

X is isobutyryl;
R₂*′ is methyl;
R₃*′ is iso-butyl;
R₅*′ is sec-butyl;
R₆*′ is 4-hydroxybenzyl, wherein the OH group is protected with a suitable protecting group;
R₇*′ is sec-butyl;
R₈*′ is 3-amino-3-oxopropyl wherein the NH₂ group of the carbamoyl is protected with a suitable protecting group; and
Y is methyl.

In another embodiment, the present invention relates to the use of the compound of formula (II′), or salt thereof, especially of the formula (II′A), or a salt thereof, for the synthesis of a compound of formula (I), or salt thereof, especially of the formula (IA), or a salt thereof.

In a further embodiment, the invention relates to a compound of formula (III′), or a salt thereof,

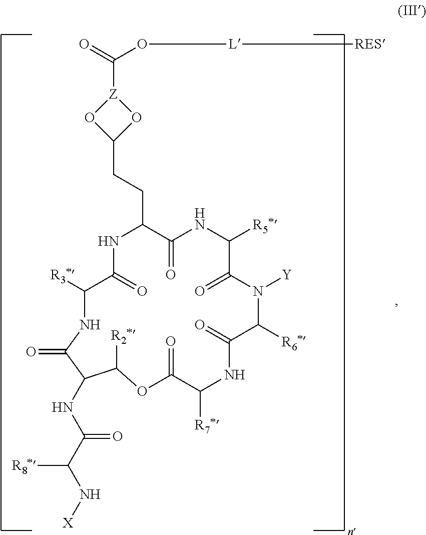

especially of the formula (III′A),

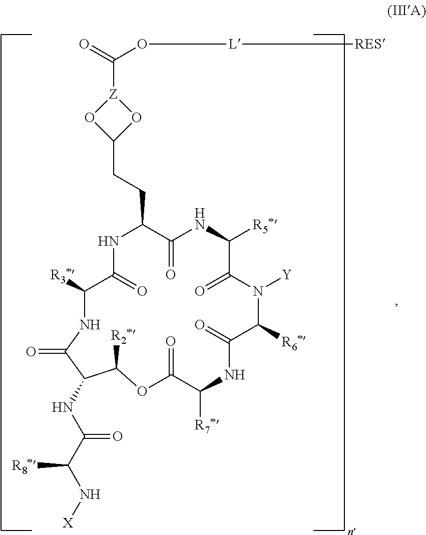

wherein Y and X are as defined for a compound of formula (I) above and Z, R₂*′, R₃*′, R₅*′, R₆*′, R₇*′ and R₈*′ are as defined for a compound of formula (II′) above,
L′ is a cleavable linker, RES′ is a solid resin and n′ is a natural number not including 0.

In a preferred embodiment, the invention relates to a compound of formula (III′), or a salt thereof, especially of the formula (III′A), or a salt thereof, wherein

X is acetyl or isobutyryl;
R₂*′ is methyl;
R₃*′ is iso-butyl, sec-butyl, or iso-propyl;
R₅*′ is benzyl, iso-butyl, sec-butyl, or iso-propyl;
R₆*′ is 4-hydroxybenzyl, wherein the OH group is protected with a suitable protecting group;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge; and
Y is methyl.

In a more preferred embodiment, the invention relates to a compound of formula (III′), or a salt thereof, especially of the formula (III′A), or a salt thereof, wherein

X is isobutyryl;
R₂*′ is methyl;
R₃*′ is iso-butyl;
R₅*′ is sec-butyl;
R₆*′ is 4-hydroxybenzyl, wherein the OH group is protected with a suitable protecting group;
R₇*′ is sec-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene; and
Y is methyl.

In another embodiment, the present invention relates to the use of the compound of formula (III′), or salt thereof, especially of the formula (III′A), or a salt thereof, for the synthesis of a compound of formula (I), or salt thereof, especially of the formula (IA), or a salt thereof.

In a further embodiment, the invention relates to a compound of formula (IV′),

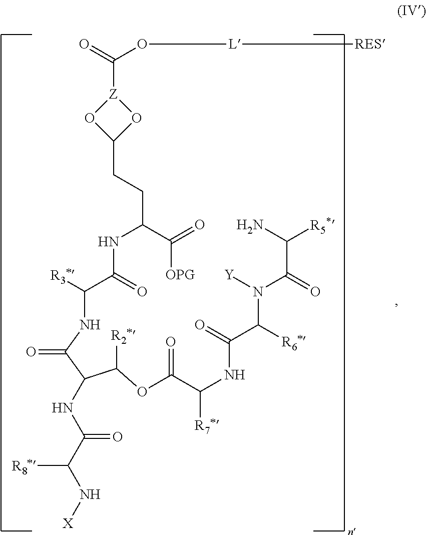

especially (IV′A),

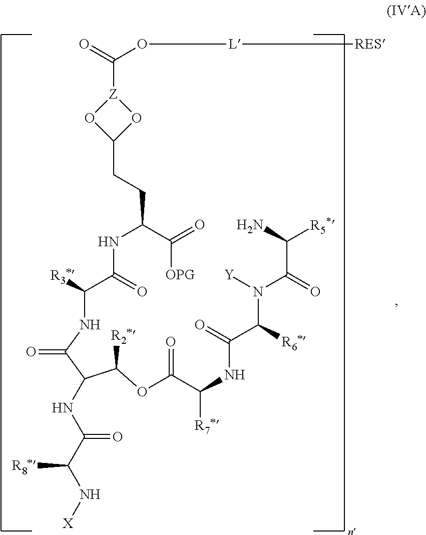

wherein Y and X are as defined for a compound of formula (I) above and Z, R₂*′, R₃*′, R₅*′, R₆*′, R₇*′ and R₈*′ are as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (IV′), especially of the formula (IV′A), wherein

X is acetyl or isobutyryl;
R₂*′ is methyl;
R₃*′ is iso-butyl, sec-butyl, or iso-propyl;
R₅*′ is benzyl, iso-butyl, sec-butyl, or iso-propyl;
R₆*′ is 4-hydroxybenzyl, wherein the OH group is protected with a suitable protecting group;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge;
Y is methyl; and
PG is allyl, 2-methyl-2-propenyl, 3-methylbut-2-enyl or 2-methylbut-3-en-2-yl.

In a more preferred embodiment, the invention relates to a compound of formula (IV′) especially of the formula (IV′A), wherein

X is isobutyryl;
R₂*′ is methyl;
R₃*′ is iso-butyl;
R₅*′ is sec-butyl;
R₈*′ is 4-hydroxybenzyl, wherein the OH group is protected with a suitable protecting group;
R₇*′ is sec-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene;
Y is methyl; and
PG is allyl

In another embodiment, the present invention relates to the use of the compound of formula (IV′), or salt thereof, especially of the formula (IV′A), or a salt thereof, for the synthesis of a compound of formula (I), or salt thereof, especially of the formula (IA), or a salt thereof.

In a further embodiment, the invention relates to a compound of formula (VI′),

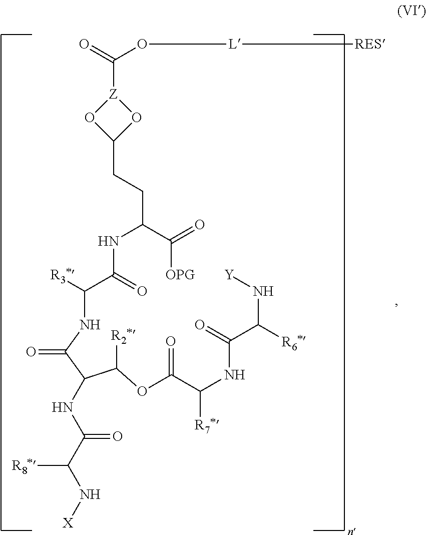

especially (VI′A),

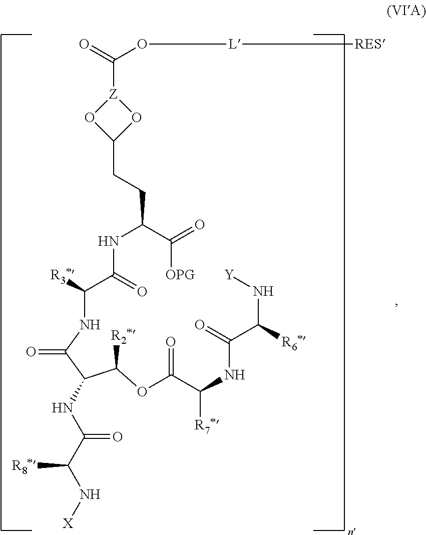

wherein Y and X are as defined for a compound of formula (I) above and Z, R₂*′, R₃*′, R₆*′, R₇*′ and R₈*′ are as defined for a compound of formula (II′) above,
L′ is a cleavable linker, RES′ is a solid resin, n′ is a natural number not including 0 and
PG is a carboxy protecting group.

In a preferred embodiment, the invention relates to a compound of formula (VI′), especially of the formula (VI′A), wherein

X is acetyl or isobutyryl;
R₂*′ is methyl;
R₃*′ is iso-butyl, sec-butyl, or iso-propyl;
R₆*′ is 4-hydroxybenzyl, wherein the OH group is protected with a suitable protecting group;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge;
Y is methyl; and
PG is allyl, 2-methyl-2-propenyl, 3-methylbut-2-enyl or 2-methylbut-3-en-2-yl.

In a more preferred embodiment, the invention relates to a compound of formula (VI′) especially of the formula (VI′A), wherein

X is isobutyryl;
R₂*′ is methyl;
R₃*′ is iso-butyl;
R₆*′ is 4-hydroxybenzyl, wherein the OH group is protected with a suitable protecting group;
R₇*′ is sec-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene;
Y is methyl; and
PG is allyl.

In another embodiment, the present invention relates to the use of the compound of formula (VI′), or salt thereof, especially of the formula (V′IA), or a salt thereof, for the synthesis of a compound of formula (I), or salt thereof, especially of the formula (IA), or a salt thereof.

In a further embodiment, the invention relates to a compound of formula (VIII′),

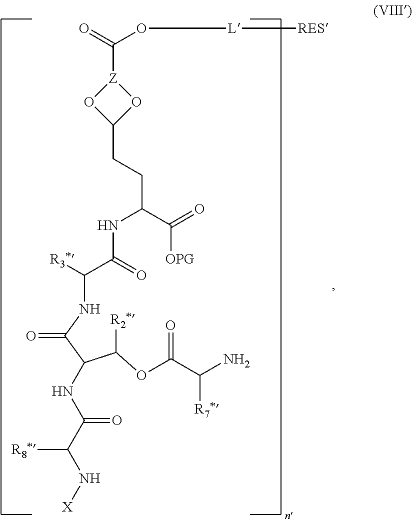

especially (VIII′A),

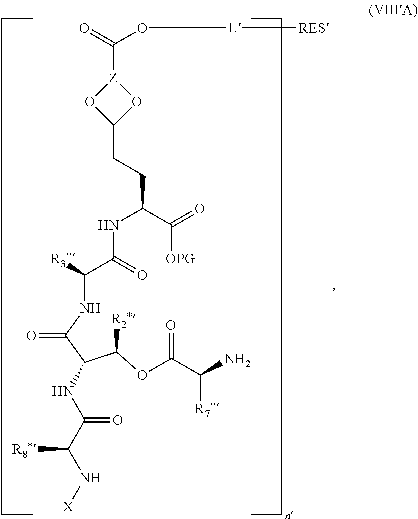

wherein Y and X are as defined for a compound of formula (I) above and Z, R₂*′, R₃*′, R₇*′ and R₈*′ are as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (VIII′), especially of the formula (VIII′A), wherein

X is acetyl or isobutyryl;
R₂*′ is methyl;
R₃*′ is iso-butyl, sec-butyl, or iso-propyl;
R₇*′ is iso-butyl, sec-butyl or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge; and
PG is allyl, 2-methyl-2-propenyl, 3-methylbut-2-enyl or 2-methylbut-3-en-2-yl.

In a more preferred embodiment, the invention relates to a compound of formula (VIII′) especially of the formula (VIII′A), wherein

X is isobutyryl;
R₂*′ is methyl;
R₃*′ is iso-butyl;
R₇*′ is sec-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl; and

RES′ is divinylbenzene crosslinked polystyrene; and
PG is allyl.

In another embodiment, the present invention relates to the use of the compound of formula (VIII′), or salt thereof, especially of the formula (VIII′A), or a salt thereof, for the synthesis of a compound of formula (I), or salt thereof, especially of the formula (IA), or a salt thereof.

In a further embodiment, the invention relates to a compound of formula (X′),

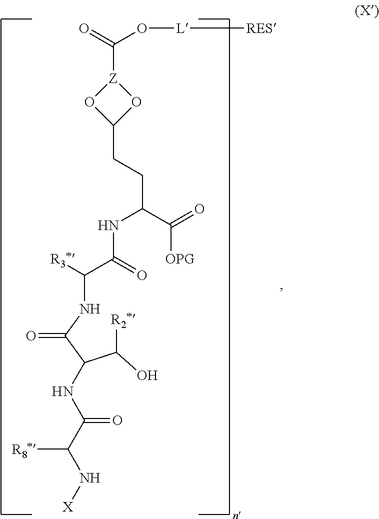

especially (X′A),

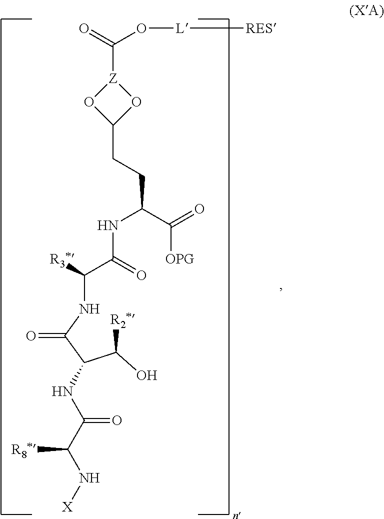

wherein X is as defined for a compound of formula (I) above and Z, R₂*′, R₃*′ and R₈*′ are as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (X′), especially of the formula (X′A), wherein

X is acetyl or isobutyryl;
R₂*′ is methyl;
R₃*′ is iso-butyl, sec-butyl, or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge; and
PG is allyl, 2-methyl-2-propenyl, 3-methylbut-2-enyl or 2-methylbut-3-en-2-yl.

In a more preferred embodiment, the invention relates to a compound of formula (X′) especially of the formula (X′A), wherein

X is isobutyryl;
R₂*′ is methyl;
R₃*′ is iso-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene; and
PG is allyl.

In another embodiment, the present invention relates to the use of the compound of formula (X′), or salt thereof, especially of the formula (X′A), or a salt thereof, for the synthesis of a compound of formula (I), or salt thereof, especially of the formula (IA), or a salt thereof.

In a further embodiment, the invention relates to a compound of formula (XI′),

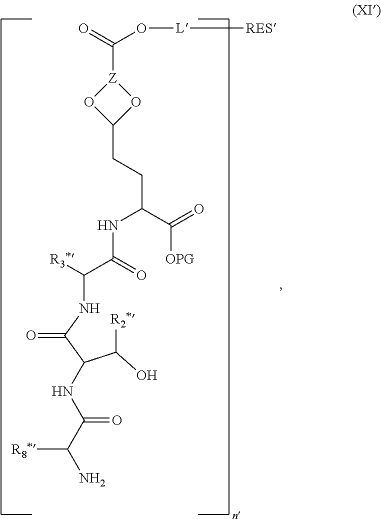

especially (XI′A),

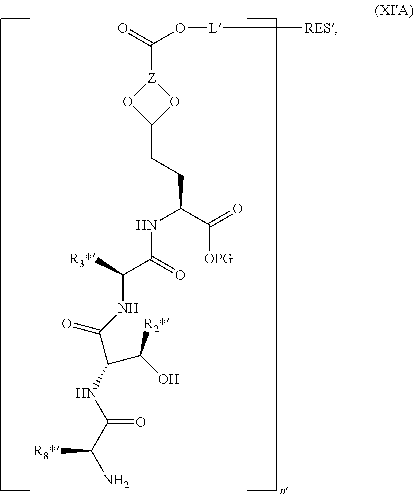

wherein Z, R₂*′, R₃*′ and R₈*′ are as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (XI′), especially of the formula (XI′A), wherein

R₂*′ is methyl;
R₃*′ is iso-butyl, sec-butyl, or iso-propyl;
R₈*′ is 2-amino-2-oxoethyl or 3-amino-3-oxopropyl wherein the NH₂ group of the carbamoyl is protected with a suitable protecting group;
Z is a —CH₂—CH₂— or —CH₂—CH₂—CH₂— bridge; and
PG is allyl, 2-methyl-2-propenyl, 3-methylbut-2-enyl or 2-methylbut-3-en-2-yl.

In a more preferred embodiment, the invention relates to a compound of formula (XI′) especially of the formula (XI′A), wherein

R₂*′ is methyl;
R₃*′ is iso-butyl;
R₈*′ is 3-amino-3-oxopropyl wherein the NH₂ group of the carbamoyl is protected with a suitable protecting group;
Z is a —CH₂—CH₂— bridge;

### L′ is 2Cl-trityl;

RES′ is divinylbenzene crosslinked polystyrene; and
PG is allyl.

In another embodiment, the present invention relates to the use of the compound of formula (XI′), or salt thereof, especially of the formula (XI′A), or a salt thereof, for the synthesis of a compound of formula (I), or salt thereof, especially of the formula (IA), or a salt thereof.

In a further embodiment, the invention relates to a compound of formula (XII′),

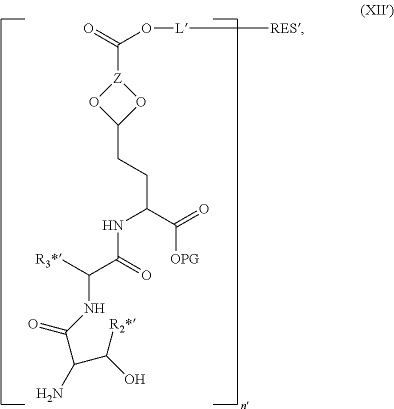

especially (XII′A)

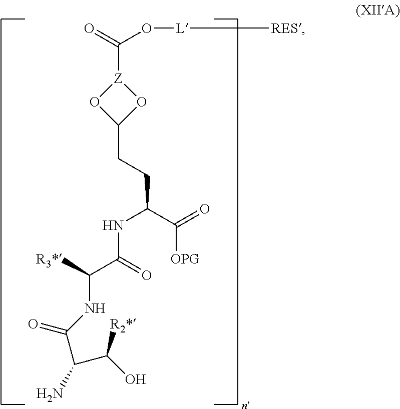

wherein Z, R₂*′ and R₃*′ are as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (XII′), especially of the formula (XII′A), wherein

R₂*′ is methyl;
R₃*′ is iso-butyl, sec-butyl, or iso-propyl;
Z is a —CH₂—CH₂— or —CH₂—CH₂—CH₂— bridge; and
PG is allyl, 2-methyl-2-propenyl, 3-methylbut-2-enyl or 2-methylbut-3-en-2-yl.

In a more preferred embodiment, the invention relates to a compound of formula (XII′) especially of the formula (XII′A), wherein

R₂*′ is methyl;
R₃*′ is iso-butyl;
Z is a —CH₂—CH₂— bridge;

### L′ is 2Cl-trityl;

RES′ is divinylbenzene crosslinked polystyrene; and
PG is allyl.

In another embodiment, the present invention relates to the use of the compound of formula (XII′), or salt thereof, especially of the formula (XII′A), or a salt thereof, for the synthesis of a compound of formula (I), or salt thereof, especially of the formula (IA), or a salt thereof.

In a further embodiment, the invention relates to a compound of formula (XIV′),

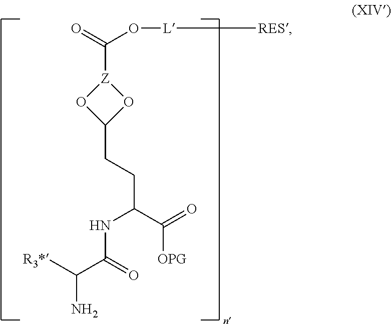

especially (XIV′A)

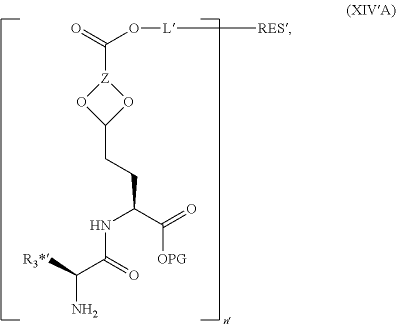

wherein Z and R₃*′ are as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (XII′), especially of the formula (XII′A), wherein

R₃*′ is iso-butyl, sec-butyl, or iso-propyl;
Z is a —CH₂—CH₂— or —CH₂—CH₂—CH₂— bridge; and
PG is allyl, 2-methyl-2-propenyl, 3-methylbut-2-enyl or 2-methylbut-3-en-2-yl.

In a more preferred embodiment, the invention relates to a compound of formula (XII′) especially of the formula (XII′A), wherein

R₃*′ is iso-butyl;
Z is a —CH₂—CH₂— bridge;

### L′ is 2Cl-trityl;

RES′ is divinylbenzene crosslinked polystyrene; and
PG is allyl.

In another embodiment, the present invention relates to the use of the compound of formula (XIV′), or salt thereof, especially of the formula (XIV′A), or a salt thereof, for the synthesis of a compound of formula (I), or salt thereof, especially of the formula (IA), or a salt thereof.

In a further embodiment, the invention relates to a compound of formula (XVI′),

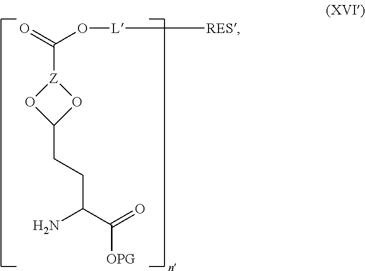

especially (XVI′A)

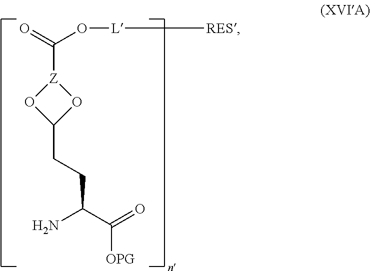

wherein Z is as defined for a compound of formula (II′) above,
L′ is a cleavable linker, RES′ is a solid resin, n′ is a natural number not including 0 and PG is a carboxy protecting group.

In a preferred embodiment, the invention relates to a compound of formula (XVI′), especially of the formula (XVI′A), wherein

Z is a —CH₂—CH₂— or —CH₂—CH₂—CH₂— bridge; and
PG is allyl, 2-methyl-2-propenyl, 3-methylbut-2-enyl or 2-methylbut-3-en-2-yl.

In a more preferred embodiment, the invention relates to a compound of formula (XVI′) especially of the formula (XVI′A), wherein

Z is a —CH₂—CH₂— bridge;

### L′ is 2Cl-trityl;

RES′ is divinylbenzene crosslinked polystyrene; and
PG is allyl.

In another embodiment, the present invention relates to the use of the compound of formula (XVI′), or salt thereof, especially of the formula (XVI′A), or a salt thereof, for the synthesis of a compound of formula (I), or salt thereof, especially of the formula (IA), or a salt thereof.

In another embodiment, the present invention relates to the use of the compound of formula (XIV′), or salt thereof, especially of the formula (XIV′A), or a salt thereof, for the synthesis of a compound of formula (I), or salt thereof, especially of the formula (IA), or a salt thereof.

In a further embodiment, the invention relates to a compound of formula (XVII′),

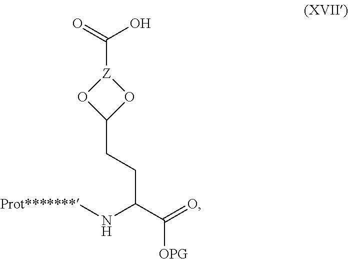

especially (XVII′A)

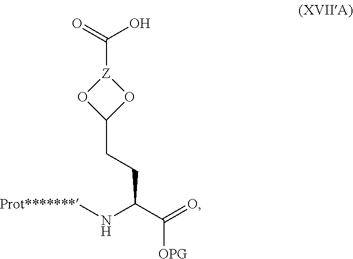

wherein Z is as defined for a compound of formula (II′) above, PG is a carboxy protecting group and Prot*******′ is an amino protecting group.

In a preferred embodiment, the invention relates to a compound of formula (XVII′), especially of the formula (XVII′A), wherein

Z is a —CH₂—CH₂— or —CH₂—CH₂—CH₂— bridge;
Prot*******′ is selected from fluoren-9-ylmethoxycarbonyl (Fmoc); 2-(2′ or 4′-pyridyl)ethoxycarbonyl and 2,2-bis(4′nitrophenyl)ethoxycarbonyl; and
PG is allyl, 2-methyl-2-propenyl, 3-methylbut-2-enyl or 2-methylbut-3-en-2-yl.

In a more preferred embodiment, the invention relates to a compound of formula (XVII′) especially of the formula (XVII′A), wherein

Z is a —CH₂—CH₂— bridge;

### Prot*******′ is Fmoc; and

PG is allyl.

In another embodiment, the present invention relates to the use of the compound of formula (XVII′), or salt thereof, especially of the formula (XVII′A), or a salt thereof, for the synthesis of a compound of formula (I), or salt thereof, especially of the formula (IA), or a salt thereof.

The following definitions (or also definitions already included above) can replace more general terms used in invention embodiments above and below in order to define further embodiments of the invention, with either one, two or more or all general terms being replaceable by the more specific terms in order to define such invention embodiments:

In all reactions, protecting gas may be used, such as nitrogen or argon, where appropriate or necessary, and the temperatures are as known to the person skilled in the art, e.g. in the range from −25° C. to the reflux temperature of the respective reaction mixture, e.g. from −20 to plus 90° C.

As used herein, “or the like” or “and the like”, refers to the fact that other alternatives to those mentioned preceding such expression are known to the person skilled in the art and may be added to those expressions specifically mentioned; in other embodiments, “or the like” and “and the like” may be deleted in one or more or all invention embodiments.

As used herein, C₁₋₉-acyl refers to C₁₋₈-alkyl-C(O)—.

In the context of X, C₁₋₈-acyl is especially acetyl or isobutyryl, preferably isobutyryl.

As used herein, C₁₋₈-alkyl refers to a fully saturated branched, including single or multiple branching, or unbranched hydrocarbon moiety having 1 to 8 carbon atoms. Representative examples of C₁₋₈-alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl.

In the context of R₂, C₁₋₈-alkyl is preferably methyl.

As used herein, C₂₋₈-alkyl refers to a fully saturated branched, including single or multiple branching, or unbranched hydrocarbon moiety having 2 to 8 carbon atoms. Representative examples of C₁₋₈-alkyl include, but are not limited to, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl.

As used herein, a “side chain of an alpha-amino acid” is selected from those of the 20 standard alpha-amino acids arginine, histidine, lysine, aspartic acid, glutamic acid, serine, threonine, asparagine, glutamine, cysteine, glycine, alanine, leucine, isoleucine, methionine, phenylalanine, tryptophan, tyrosine, valine and proline (then with internal cyclization including the alpha-amino group).

For the alpha-amino acids, either their names or the customary three letter codes are used in the present disclosure, in accordance with the following table:

Table A

| Amino acid | Three letter code |
|---|---|
| Alanine | Ala |
| Arginine | Arg |
| Asparagine | Asn |
| Aspartic acid | Asp |
| Cysteine | Cys |
| Glutamic acid | Glu |
| Glutamine | Gln |
| Glycine | Gly |
| Histidine | His |
| isoleucine | Ile |
| Leucine | Leu |
| Lysine | Lys |
| Methionine | Met |
| Phenylalanine | Phe |
| Proline | Pro |
| Serine | Ser |
| Threonine | Thr |
| Tryptophan | Try |
| Tyrosine | Tyr |
| Valine | Val |

R₂, R₂* and R₂*′ are C₁₋₈-alkyl, preferably methyl wherever mentioned.

R₃ is the side chain of an alpha-amino acid, preferably iso-butyl (side chain of leucine), sec-butyl (side chain of isoleucine) or iso-propyl (side chain of valine), more preferably iso-butyl.

R₃* and R₃*′ are the side chain corresponding to R₃ in protected form if a reactive functional group is present that could participate in undesired side reactions. R₃* and R₃*′ are preferably iso-butyl (side chain of leucine), sec-butyl (side chain of isoleucine) or iso-propyl (side chain of valine), more preferably iso-butyl.

R₅ is the side chain of an alpha-amino acid, preferably benzyl (side chain of phenylalanine), iso-butyl (side chain of leucine), sec-butyl (side chain of isoleucine) or iso-propyl (side chain of valine), more preferably sec-butyl, especially (S)-sec-butyl.

R₅* and R₅*′ are the side chain corresponding to R₅ in protected form if a reactive functional group is present that could participate in undesired side reactions. R₅* and R₅*′ are preferably benzyl (side chain of phenylalanine), iso-butyl (side chain of leucine), sec-butyl (side chain of isoleucine) or iso-propyl (side chain of valine), more preferably sec-butyl, especially (S)-sec-butyl.

R₆ is the side chain of an alpha-amino acid, wherein the side chain contains a hydroxy group, preferably of tyrosine.

R₆* and R₆*′ are the side chain corresponding to R₆ in protected form if a reactive functional group is present that could participate in undesired side reactions. R₆* and R₆*′ are preferably 4-hydroxybenzyl (side chain of tyrosine), wherein the OH group is protected with a suitable protecting group.

R₇ is the side chain of an alpha-amino acid, preferably iso-butyl (side chain of leucine), sec-butyl (side chain of isoleucine) or iso-propyl (side chain of valine), more preferably sec-butyl, especially (S)-sec-butyl.

R₇* and R₇*′ are the side chain corresponding to R₇ in protected form if a reactive functional group is present that could participate in undesired side reactions. R₇* and R₇*′ are preferably iso-butyl (side chain of leucine), sec-butyl (side chain of isoleucine) or iso-propyl (side chain of valine), more preferably sec-butyl, especially (S)-sec-butyl.

R₈ is the side chain of an alpha-amino acid, wherein the side chain contains a terminal carboxy or carbamoyl group, preferably 2-amino-2-oxoethyl (side chain of asparagine) or 3-amino-3-oxopropyl (side chain of glutamine), more preferably 3-amino-3-oxopropyl.

R₈* and R₈*′ are the side chain corresponding to R₈ in protected form if a reactive functional group is present that could participate in undesired side reactions. R₈* and R₈*′ are preferably 2-amino-2-oxoethyl (side chain of asparagine) or 3-amino-3-oxopropyl (side chain of glutamine), wherein the NH₂ group of the carbamoyl is protected with a suitable protecting group; more preferably 3-amino-3-oxopropyl, wherein the NH₂ group of the carbamoyl is protected with a suitable protecting group.

As used herein, a “reactive functional group” that could participate in undesired side reactions is a functional group present in a “side chain of an alpha-amino acid” such as an amino group (side chain of glycine, histidine, lysine, tryptophane), a hydroxy group (side chain of serine, threonine, tyrosin), a carboxy group (side chain of aspartic acid, glutamic acid), a sulfhydryl group (side chain of cysteine), a carbamoyl group (side chain of asparagine, glutamine) or a guanidine group (side chain of arginine), As used herein, “in protected form” mean protected with a suitable protecting group.

Appropriate “suitable protecting groups” are known in the art, as well methods for their introduction and removal. Such protecting groups are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

Y is hydrogen or C₁₋₈-alkyl, preferably methyl.

Rk and Rl are each independently of each other linear or branched C₁₋₈-alkyl or benzyl, preferably benzyl;

or Rk and Rl together form a linear or branched C₁₋₈-alkylene bridge, so that Rk and Rl together with the two oxygen atoms and the carbon atom to which the two oxygen atoms are bound, form a 5-7 membered ring; preferably, Rk and Rl together form a —CH₂—CH₂— or —CH₂—CH₂—CH₂— bridge;
more preferably, Rk and Rl form a —CH₂—CH₂— bridge

All of the compounds can—where salt-forming groups such as basic groups, e.g. amino or imino, or acidic groups, e.g. carboxyl or phenolic hydroxyl, are present—be used in free form or as salts or as mixtures of salts and free forms. Thus where ever a compound is mentioned, this includes all these variants. For example, basic groups may form salts with acids, such as hydrohalic acids, e.g. HCl, sulfuric acid or organic acids, such as acetic acid or trifluoroacetic acid, while acidic groups may form salts with positive ions, e.g. ammonium, alkylammonium, triethylamine, N-methylmorpholine, dimethylaminopyridine, alkali or alkaline-earth metal salt cations, e.g. Ca, Mg, Na, K or Li cations, or the like, or zwitterionic salts or inner salts of the compounds may be present.

As used herein an “amino protecting group” refers to a protecting group conventionally used in peptide chemistry. Such protecting groups are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

Examples of preferred amino protecting groups are acetyl, benzyl, cumyl, benzhydryl, trityl, benzyloxycarbonyl (Cbz), 9-fluorenylmethyloxycarbony (Fmoc), benzyloxymethyl (BOM), pivaloyl-oxy-methyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilyethoxymethyl (SEM), t-butoxycarbonyl (BOC), t-butyl, 1-methyl-1, 1-dimethylbenzyl, (phenyl)methylbenzene, pyrridinyl and pivaloyl. More preferred nitrogen protecting groups are acetyl, benzyl, benzyloxycarbonyl (Cbz), 9-fluorenylmethyloxycarbony (Fmoc), triethylsilyl (TES), trimethylsilyethoxymethyl (SEM), t-butoxycarbonyl (BOC), pyrrolidinylmethyl and pivaloyl.

As used herein a “carboxy protecting group” refers to refers to a protecting group conventionally used to protect a carboxy group. Such protecting groups are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

Examples of preferred carboxy protecting groups are C₁₋₈-alkyl such as methyl, ethyl or t-butyl; benzyl; allyl; 2-methyl-2-propenyl; 3-methylbut-2-enyl; 2-methylbut-3-en-2-yl; trimethylsilyl (TMS); tert.-butyldimethylsilyl (TBDMS); triethylsilyl (TES); triisopropylsilyl; trimethylsilyethoxymethyl (SEM) and benzyloxymethyl (BOM). Most preferred carboxy protecting groups are methyl, ethyl t-butyl, benzyl, allyl, 2-methyl-2-propenyl or 3-methylbut-2-enyl.

The protecting groups Prot*, Prot**, Prot***, Prot****, Prot*****, Prot******, Prot*******, Prot*′, Prot**′, Prot***′, Prot****′, Prot*****′, Prot******′, Prot*******′ are independently of each other amine protecting groups as defined above. Examples of preferred amino protecting groups for Prot*, Prot**, Prot***, Prot****, Prot*****, Prot******, Prot*******, Prot*′, Prot**′, Prot***′, Prot****′, Prot*****′, Prot******′, Prot*******′ are acetyl, benzyl, cumyl, benzhydryl, trityl, benzyloxycarbonyl (Cbz), 9-fluorenylmethyloxycarbony (Fmoc), benzyloxymethyl (BOM), pivaloyl-oxy-methyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilyethoxymethyl (SEM), t-butoxycarbonyl (BOC), t-butyl, 1-methyl-1, 1-dimethylbenzyl, (phenyl)methylbenzene, pyrridinyl and pivaloyl. More preferred nitrogen protecting groups for Prot*, Prot**, Prot***, Prot****, Prot*****, Prot******, Prot*******, Prot*′, Prot**′, Prot***′, Prot****′, Prot*****′, Prot******′, Prot*******′ are acetyl, benzyl, benzyloxycarbonyl (Cbz), 9-fluorenylmethyloxycarbony (Fmoc), triethylsilyl (TES), trimethylsilyethoxymethyl (SEM), t-butoxycarbonyl (BOC), pyrrolidinylmethyl and pivaloyl. Most preferably, the protecting groups Prot*, Prot**, Prot***, Prot****, Prot*****, Prot******, Prot*******, Prot*′, Prot**′, Prot***′, Prot****, Prot*****′, Prot******′, Prot*******′ are fluorenylmethyloxycarbony (Fmoc).

The protecting groups on the moieties R₂*, R₃*, R₅*, R₆*, R₇* or R₈* or R₂*′, R₃*′, R₅*′, R₆*′, R₇*′ or R₈*′ are chosen from suitable protecting groups known in the art, with the proviso that these protecting groups are chosen so that these protecting groups are conserved under the conditions of SPPS, especially the conditions for removal of Prot*, Prot**, Prot***, Prot****, Prot*****, Prot****** and Prot******* or Prot*′, Prot**′, Prot***′, Prot****′, Prot*****′, Prot******′ and Prot*******′, respectively. Such protecting groups are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/1, Georg Thieme Verlag, Stuttgart 1974.

The protecting groups selected from the group consisting of Prot*, Prot**, Prot***, Prot****, Prot*****, Prot****** or Prot*******; and protecting groups selected from the group consisting of any protecting groups present on the moieties R₂*, R₃*, R₅*, R₆*, R₇* or R₈* where ever mentioned throughout the present description and claims, are selected so that they allow for orthogonal protection.

The protecting groups selected from the group consisting of Prot*′, Prot**′, Prot***′, Prot****′, Prot*****′, Prot******′ or Prot*******′ and protecting groups selected from the group consisting of any protecting groups present on the moieties R₂*′, R₃*′, R₅*′, R₆*′, R₇*′ or R₈*′ where ever mentioned throughout the present description and claims, are selected so that they allow for orthogonal protection.

Orthogonal protection is a strategy allowing the deprotection of multiple protective groups one (or more but not all) at the time where desired each with a dedicated set of reaction conditions without affecting the other protecting group(s) or bonds to resins, e.g. via linkers on solid synthesis resins. In other terms: The strategy uses different classes of protecting groups that are removed by different chemical mechanisms, also using appropriate linkers and connectors in the case of solid phase peptide synthesis (where the linker-resin bond might be considered as a carboxy protecting group).

The protecting groups Prot*, Prot**, Prot***, Prot****, Prot*****, Prot****** and Prot*******, or Prot*′, Prot**′, Prot***′, Prot****′, Prot*****′, Prot******′ and Prot*******′ and the protecting groups present on the moieties R₂*, R₃*, R₅*, R₆*, R₇* and R₈* or R₂*′, R₃*′, R₅*′, R₆*′, R₇*′ and R₈*′ respectively, are thus not limited to those mentioned above—rather they should fulfill conditions that make them appropriate for orthogonal protection.

The preferred orthogonal synthesis method in the present invention makes use of the Fmoc-protecting group strategy known in general for peptide synthesis using solid phase and liquid phase peptide synthesis. When using this strategy, it is recommended to avoid too basic conditions (though the bases described for Fmoc cleavage, such as piperidine, are usually allowable) to avoid cleavage of the depsipeptide (ester) bond.

Where Prot*, Prot**, Prot***, Prot****, Prot*****, Prot******, Prot*******, Prot*′, Prot**′, Prot***′, Prot****, Prot*****′, Prot******′, Prot*******′ are fluorenylmethyloxycarbony (Fmoc), a suitable protecting group on the R₆* and R₆*′ moiety is especially t-butyl and a suitable protecting group on the R₈* and R₈*′ moiety is especially trityl.

PG is a suitable carboxy protecting group as defined above with the proviso that this protecting group is chosen so that it is conserved under the conditions of SPPS, especially the conditions for removal Prot*′, Prot**′, Prot***′, Prot****′, Prot*****′, Prot******′ and Prot*******′. Preferred suitable carboxy protecting group are allyl, benzyl, 2-methyl-2-propenyl, 3-methylbut-2-enyl or 2-methylbut-3-en-2-yl More preferred are allyl, 2-methyl-2-propenyl, 3-methylbut-2-enyl or 2-methylbut-3-en-2-yl.

PG₁ and PG₂, are independently of each other amine protecting groups as defined above. In a preferred embodiment, PG₁ and PG₂, are the same. In a more preferred embodiment, PG₁ and PG₂, are benzyl.

PG₃ is a carboxy protecting group as defined above. In a preferred embodiment, PG₃ is benzyl.

PG₄ is a carboxy protecting group as defined above. In a preferred embodiment, PG₃ is C₁₋₈-alkyl, more preferably methyl.

Acetals are highly sensitive to acidic conditions, especially in the presence of water. Cleavage of the acetal groups during the solid phase peptide synthesis or during cleavage from solid support would generate the free aldehyde function, which could react with the free amino group and undergo other side reactions. Therefore, it is important to ensure the conservation of the acetal groups until the macrolactamization step performed to obtain the macrocyclic compound of formula (II) or (IIA) or of the formula (II′) or (II′A) respectively.

As used herein, “solid resin” refers to a solid resin for Solid Phase Peptide Synthesis (SPPS) such as:
Gel-type supports without or with spacer: These are highly solvated polymers with an equal distribution of functional groups. This type of support is the most common, and includes:
   Polystyrene: Styrene cross-linked with e.g. 1-2% divinylbenzene; Polyacrylamide or polymethacrylamide: as hydrophilic alternative to polystyrene; Polyethylene glycol (PEG): PEG-Polystyrene (PEG-PS) is more stable than polystyrene and spaces the site of synthesis from the polymer backbone; PEG-based supports: Composed of a PEG-polypropylene glycol network or PEG with polyamide or polystyrene (these already include a spacer, PEG);
Surface-type supports: Materials developed for surface functionalization, including controlled pore glass, cellulose fibers, and highly cross-linked polystyrene.
Composites: Gel-type polymers supported by rigid matrices.

Usually these gels carry reactive groups to which a linker L as mentioned for various precursors above and below can be bound. For example, such groups include aminomethyl groups, polyethyleneglycol groups with a terminal hydroxy, and the like. Preferably, solid resins for SPPS are gel type supports, examples of such resins are divinylbenzene crosslinked polystyrene; polyacrylamide and polymethacrylamide; more preferably divinylbenzene crosslinked polystyrene.

As used herein “cleavable linker” refers to all commonly known and appropriate cleavable linkers customarily used in SPPS. Examples of such linkers are the 2-methoxy-4-benzyloxybenzyl alcohol linker (a Sasrin-Linker, Sasrin stands for superacid sensitive resin, binds the amino acids or peptides via alcoholic OH); the trityl linker family (e,g, trityl, 2Cl-trityl, which bind the amino acids or peptides via OH); the 4-(2,4-dimethoxyphenylhydroxymethyl)phenoxymethyl-Linker (Rink-Acid-Linker, binds the amino acids or peptides via OH); or tris(alkoxy)benzyl ester linkers (HAL-Linker, binds the amino acids or peptides via OH). Preferably, cleavable linkers

Where reactive derivatives of acids, especially amino acids, or peptides, e.g. dipeptides, are mentioned, they may be formed in situ or may be used as such.

As used herein, “coupling agent” refers to a coupling agent or reagent for producing an activated ester that are known to the person skilled in the art and can be deduced conveniently from many sources, e.g. Aldrich ChemFiles—Peptide Synthesis (Aldrich Chemical Co., Inc., Sigma-Aldrich Corporation, Milwaukee, Wis., USA) Vol. 7 No. 2, 2007 (see http://www.sigmaaldrich.com/etc/medicalib/docsAldrich/Brochure/al_chemfile_v7_n2.Par. 0001.File.tmp.al_chemfile_v7_n2.pdf). Examples of coupling agents include:
Cyanooxim derivatives, e.g. ethyl 2-cyano-2-(hydroxyimino)acetate (Oxima), ethyl 2-cyano-2-(naphthalen-2-ylsulfonyloxyimino)acetate (NpsOXY), ethyl 2-cyano-2-(tosyloxyimino)acetate (TsOXY), O-[(cyano(ethoxycarbonyl)methyliden)amino]-, 1,3,3-tetramethyluronium-tetrafluoroborate (TOTU), 1-cyano-2-ethoxy-2-oxoethylideneaminooxy-dimethylamino-morpholino-carbenium hexafluorophosphate (HOTU), ethyl (hydroxyimino)cyanoacetate (COMU), O-[(1-cyano-2-ethoxy-2-oxoethylidene)amino]-oxytri(pyrrolidin-1-yl) phosphonium tetrafluoroborate (PyOxB), O-[(1-cyano-2-ethoxy-2-oxoethylidene)amino]-oxytri(pyrrolidin-1-yl) phosphonium hexafluorophosphate (PyOxP)
Triazoles, uronium or hexafluorophosponium derivatives, e.g. 1-hydroxy-benzotriazole (HOBt), 1-hydroxy-7-aza-benzotriazole (HOAt), ethyl 2-cyano-2-(hydroxyimino)acetate, 2-(1H-7-Azabenzotriazol-1-yl)-1,1,3,3-tetramethyluronium hexafluorophosphate methanaminium (HATU), benzotriazol-1-yl-oxytripyrrolidinophosphonium hexafluorophosphate (PyBOP), 1-(mesitylene-2-sulfonyl)-3-nitro-1,2,4-triazole (MSNT), 2-(1H-benzotriazol-1-yl)-1,1,3,3-tetramethyluronium-hexafluorophosphate (HBTU), 2-(1H-benzotriazol-1-yl)-1,1,3,3-tetramethyluronium-hexafluoroborate (TBTU), 2-succinimido-1,1,3,3-tetramethyl-uronium-tetrafluoroborate (TSTU), 2-(5-norbornen-2,3-dicarboximido)-1,1,3,3-tetramethyluronium-tetrafluoroborate (TNTU), O-(benzotriazol-1-yl)-1,3-dimethyl-1,3-dimethylene uronium hexafluorophosphate (HBMDU), O-(benzotriazol-1-yl)-1,1,3,3-bis(tetramethylene)uronium hexafluorophosphate (HBPyU), O-(benzotriazol-1-yl)-1,1,3,3-bis(pentamethylene)uronium hexafluorophosphate (HBPipU), 3-hydroxy-4-oxo-3,4-dihydro-1,2,3-benzotriazine (HODhbt), 1-hydroxy-7-azabenzotriazole and its corresponding uronium or phosphonium salts, designated HAPyU and AOP, chlorotripyrrolidinophosphonium hexafluorophosphate (PyCloP), or the like;
Carbodiimides, e.g. dicyclohexylcarbodiimide, N-(3-dimethylaminopropyl)-N′-ethylcarbodiimide, 1-tert-butyl-3-ethylcarbodiimide, N-cyclohexyl-N′-2-morpholinoethyl)carbodiimide or diisopropylcarbodiimide (DICI—especially for ester formation via O-acyl urea formation of the carboxylic group); or
Active ester forming agents, e.g. 2-mercaptobenzothiazole (2-MBT),
Acide forming agents, e.g. diphenyl phosphoryl azide,
Acid anhydrides, such as propane phosphonic acid anhydride,
Acid halogenation agents, e.g. 1-chloro-N,N,2-trimethyl-1-propenylamine, chloro-N,N, N′,N′-bis(tetramethylene)formamidinium tetrafluoroborate or hexafluorophosphate, chloro-N,N,N′,N′-tetramethlformamidinium hexafluorophosphate, fluoro-N,N,N′,N′-tetra-metylformamidinium hexafluorophosphate, fluoro-N,N,N′,N′-bis(tetramethylene)formamidinium hexafluorophosphate,
   or the like, or mixtures of two or more such agents.

As used herein, a “mild base” refers to a suitable mild base, such as piperidine, morpholine, dicyclohexylamine, p-dimethylamino-pyridine, diisopropylamine, piperazine, tris-(2-aminoethyl)amine or 4-methylpiperidine.

### EXAMPLES

### The following examples illustrate the invention without limiting its scope

### Abbreviations

aq. aqueous
Boc/BOC tert-butoxycarbonyl
brine sodium chloride solution in water (saturated at rt)
Bzl benzyl
COMU 1-cyano-2-ethoxy-2-oxoethylideneaminooxy-dimethylamino-mor-pholino-carbenium hexafluorophosphate
DCM dichloromethane
DICI diisopropyl carbodiimide
DIPEA N,N-diisopropylethylamine
DMAP 4-Dimethylaminopyridine
DMF N,N-dimethylformamide
Fmoc/FMOC 9-fluorenymethoxycarbonyl
Fmoc-HOSU-Ester 9H-fluoren-9-yl)methyl 2,5-dioxopyrrolidin-1-yl carbonate
Fmoc-OSu N-(9-fluorenylmethoxycarbonyloxy)succinimide
Et ethyl
EtOAc ethyl acetate
eq equivalent
h hour(s)
HATU 2-(1H-7-azabenzotriazol-1-yl)-1,1,3,3-tetramethyluronium hexafluorophosphate methanaminium
HFIP hexafluoroisopropanol
HOSU N-hydroxysuccinimide
HPLC High Performance Liquid Chromatography
HR-MS High Resolution Mass Spectroscopy
IPA isopropyl alcohol
IPC In-Process Control
IR Infrared Spectroscopy
IT internal temperature
Kaiser test Ninhydrin-based test to monitor deprotection in SPPS (see E. Kaiser, R. L. Colescott, C. D. Bossinger, P. I. Cook, Analytical Biochemistry 34 595 (1970)); if mentioned to be OK, this means successful deprotection.
l liter
Me methyl
MeOH methanol
MED Dichloromethane
min minute(s)
MS Mass Spectroscopy
MSNT 1-(mesitylene-2-sulfonyl)-3-nitro-1,2,4-triazole
NH₄OAc ammonium acetate
NMI N-methylimidazole
NMP N-methylpyrrolidone
NMR Nuclear Magnetic Resonance Spectroscopy
Oxyma ethyl 2-cyano-2-(hydroxyimino)acetate
Pd(PPh₃)₄ tetrakis(triphenylphosphine)palladium(0)
PyBOP benzotriazol-1-yl-oxytripyrrolidinophosphonium hexafluorophosphate
RBF round bottom flask
RP Reversed Phase
RT/rt room temperature
SPPS Solid Phase Peptide Synthesis
TBME tert-butyl methyl ether
TFA trifluoroacetic acid
TFE 2,2,2-trifluoroethanol
THF tetrahydrofuran
TLC thin layer chromatography

For amino acid abbreviations see the table above.

If not mentioned otherwise, reactions are carried out at room temperature.

The synthesis of the compound A mentioned below is made in solution according to the following simplified scheme, more details are given below:

### Example 1

### Synthesis of Compound A ((S)—N1-((1S,2S,5S,8S,11R,12S,15S,18S,21R)-2,8-di((S)-sec-butyl)-21-hydroxy-5-(4-hydroxybenzyl)-15-isobutyl-4,11-dimethyl-3,6,9,13,16,22-hexaoxo-10-oxa-1,4,7,14,17-pentaazabicyclo[16.3.1]docosan-12-yl)-2-isobutyramidopentanediamide) via macrolactamization in solution

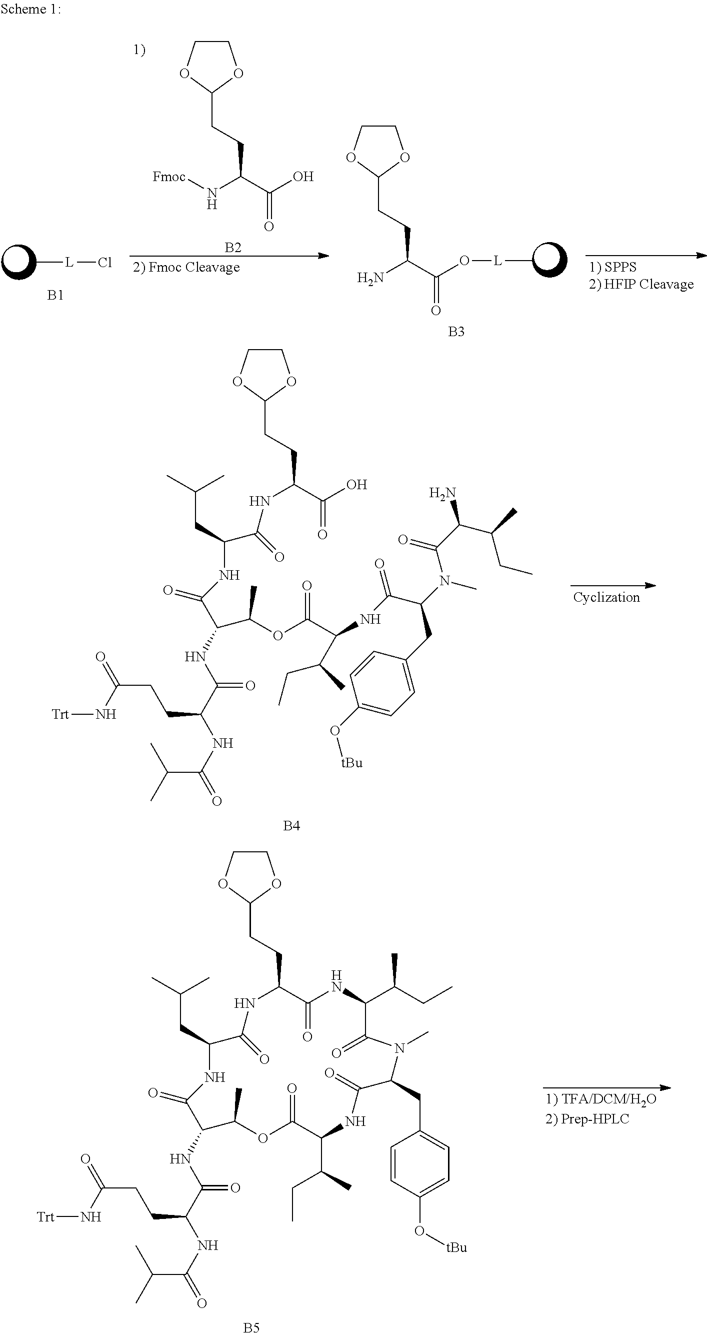

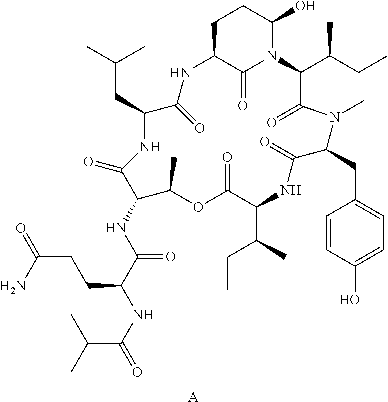

### Example 1.1

### Resin Loading—Synthesis of B3

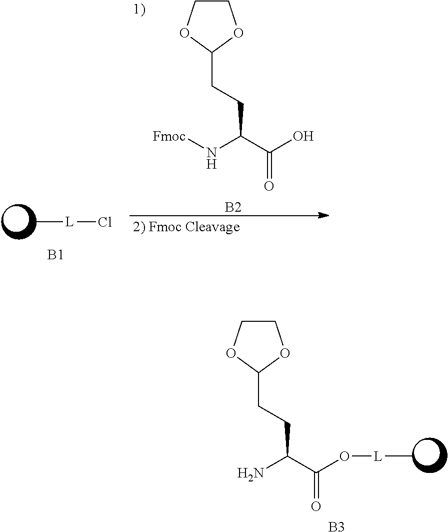

In a 100 ml solid phase synthesis reactor were added 10.0 g of 2-chlorotrityl chloride resin (L=2-chlorotrityl, ‘bead’ (RES)=1.0% divinylbenzene cross-linked polystyrene) B1 (loading=1.7 mmol/g; 17.0 mmol) and 80 ml of DCM. The suspension was stirred for 5 min and then the solvent was drained. The same washing was repeated once. The wet resin was kept aside under nitrogen atmosphere, in the meantime in a RBF were mixed 10.1 g of (S)-2-((((9H-fluoren-9-yl)methoxy)carbonyl)amino)-4-(1,3-dioxolan-2-yl)butanoic acid (B2) (25.5 mmol; 1.5 eq), 27 ml of DCM and 8.8 ml of DIPEA (51.2 mmol, 3.0 eq) and the mixture was stirred at rt until a clear solution was obtained. This solution was added in one portion to the solid phase reactor and then the mixture was stirred for 5.0 min and then a second portion of 5.8 ml of DIPEA (34 mmol, 2.0 eq) was added to the solid phase reactor. The suspension was stirred for 18 h at rt and then the reaction mixture was drained and the resin was washed twice with 35 ml of a 5% v/v solution of DIPEA in DCM.

Potential unreacted resin sites were quenched by the treatment of the resin with 80 ml of a solution of DCM/MeOH/DIPEA (70/15/15), v/v/v. At this point a sample was taken for loading measurement.

The Fmoc protecting group was cleaved treating the resin with 35 ml of a 25% v/v solution of piperidine in DMF following the general procedure for Fmoc cleavage as described below. The resin was dried under vacuum at 35° C. until constant weight and then 12.9 g of peptide resin B3 were obtained, the loading this resin calculated to be 1.04 mmol/g expressed as free amine.

### Example 1.2

### Solid Phase Peptide Synthesis (SPPS) and Cleavage—Synthesis of B4 ((2S,5S,8S,9R,12S,15S,18S,19S)-2-(2-(1,3-dioxolan-2-yl)ethyl)-18-amino-15-(4-(tert-butoxy)benzyl)-12-((S)-sec-butyl)-5-isobutyl-8-((S)-2-isobutyramido-5-oxo-5-(tritylamino)pentanamido)-9,16,19-trimethyl-4,7,11,14,17-pentaoxo-10-oxa-3,6,13,16-tetraazahenicosan-1-oic acid)

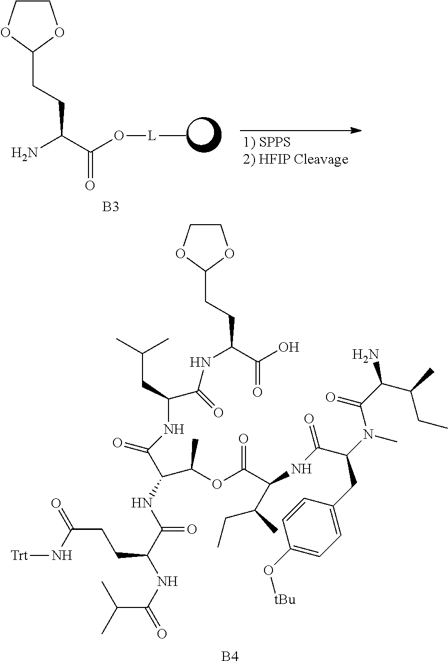

To a 50 ml solid phase peptide synthesis reactor were added 4.8 g of peptide resin B3 (loading=1.04 mmol/g; 5.0 mmol) then the resin was washed with 40 ml of DMF (2×30 min). The resin was subjected to manual solid phase peptide synthesis. The sequence of the peptide was generated with the sequential execution of the cycles* as shown in Table 1 * A cycle is defined by the execution of following 2 sequential operations (In the case of cycle 4 the Fmoc cleavage is not applied due the lack of an Fmoc group).

1. Amino acid coupling (See general procedures below)

2. Fmoc cleavage (See general procedures below)

TABLE 1
Coupling sequence and coupling conditions

| Cycle number | Amino Acid | Coupling Method |
|---|---|---|
| 1 | Fmoc-Leu-OH | HATU/DIPEA |
| 2 | Fmoc-Thr-OH | DICI/Oxyma |
| 3 | Fmoc-Gln(Trt)-OH | DICI/Oxyma |
| 4 | Isobutyric acid | DICI/Oxyma |
| 5 | Fmoc-Ile-OH | MSNT/NMI |
| 6 | Fmoc-MeTyr(tBu)-OH | HATU/DIPEA |
| 7 | Fmoc-Ile-OH | HATU/DIPEA |

After cycle number 7 was finished the peptide resin was washed additionally with 40 ml of isopropanol (3×2 min) and with 50 ml of DCM (2×2 min).

To the wet resin 200 ml of a 30% HFIP in DCM (v/v), were added in one portion. The suspension was stirred for 10 min and then the solvent was drained and collected in a 500 ml RBF, the cleavage process was repeated twice and the drained solutions were pooled. The pooled solution was concentrated under vacuum at 35° C. until an oily residue was obtained; to this oil 25 ml of toluene were added. The toluene solution was added dropwise to 300 ml of pre-cooled (0-5° C.) heptane, the suspension was stirred for 30 min, filtered with a sintered glass filter (porosity G3) and the filter cake was washed with 25 ml of pre-cooled (0-5° C.) heptane. The wet filter cake was dried under vacuum at 35° C. until constant weight and then 9.6 g of crude peptide B4 were obtained.

### Example 1.3

### Cyclisation in solution—Synthesis of B5 ((S)—N1-((3S,6S,9S,12S,15S,18S,19R)-12-(2-(1,3-dioxolan-2-yl)ethyl)-6-(4-(tert-butoxy)benzyl)-3,9-di((S)-sec-butyl)-15-isobutyl-7,19-dimethyl-2,5,8,11,14,17-hexaoxo-1-oxa-4,7,10,13,16-pentaazacyclononadecan-18-yl)-2-isobutyramido-N5-tritylpentanediamide)

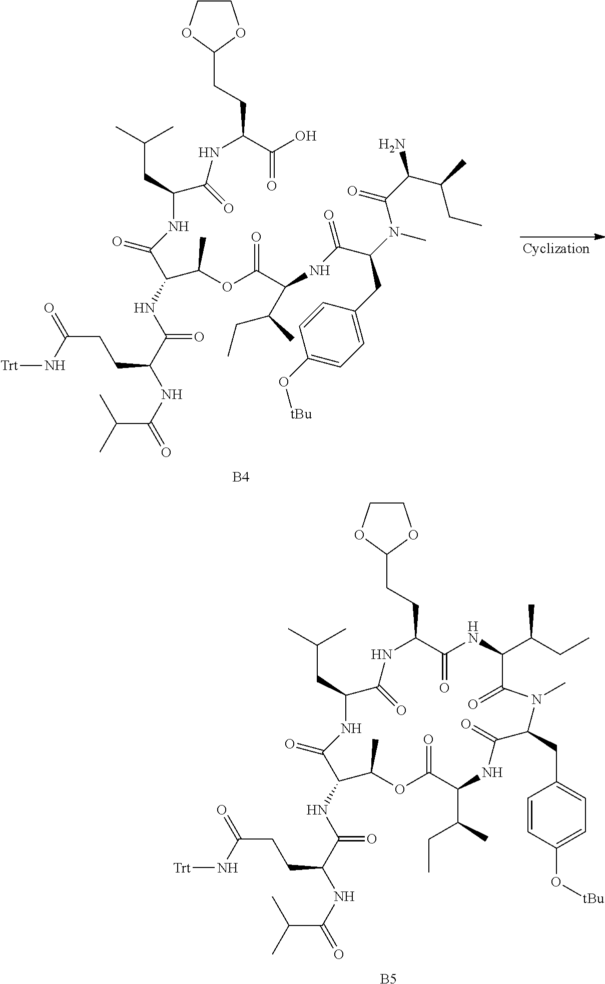

In a RBF 6.4 g of B4 (5.0 mmol, 1.0 eq) and 3.8 g of HATU were dissolved in 500 ml of DMF then 3.5 ml of DIPEA were added. The reaction mixture was stirred for 20 min and then 1.0 l of brine and then 100 ml of pure water. The suspension was filtered using a sintered glass filter (G3); the filter cake was washed with water and the crude peptide purified by silica gel filtration to yield 2.1 g of B5.

HR-MS: Calculated for C₇₁H₉₈N₈O₁₃ [M+H]+: 1271.73261; [M+NH4]+: 1288.75916; [M+Na]+: 1293.71456. Found: [M+H]+: 1271.7328; [M+NH₄]+: 1288.7600.

¹H-NMR (600 MHz, d₆-DMSO): δ ppm 0.47 (3H, m); 0.73 (3H, m); 0.75-0.87 (12H, m); 0.97-1.05 (8H, m); 1.10 (3H, m); 1.13 (1H, m); 1.25 (9H, m); 1.33 (1H, m); 1.46 (1H, m); 1.56 (6H, m); 1.65 (2H, m); 1.79 (1H, m); 1.96 (1H, m); 2.30 (2H, m); 2.43 (1H, m); 2.68 (4H, m); 3.29 (1H, m); 3.73-3.88 (4H, m); 4.17 (1H, m); 4.19 (1H, m); 4.24 (1H, m); 4.30 (1H, m); 4.42 (1H, m); 4.57 (1H, m); 4.73 (1H, m); 5.17 (1H, m); 5.26 (1H, m); 6.86 (2H, m); 7.12-7.27 (18H, m); 7.90 (1H, m); 7.94 (1H, m); 8.18 (1H, m); 8.51 (2H, m); 8.82 (1H, m).

### Example 1.4

### Synthesis of A ((S)—N1-((1S,2S,5S,8S,11R,12S,15S,18S,21R)-2,8-di((S)-sec-butyl)-21-hydroxy-5-(4-hydroxybenzyl)-15-isobutyl-4,11-dimethyl-3,6,9,13,16,22-hexaoxo-10-oxa-1,4,7,14,17-pentaazabicyclo[16.3.1]docosan-12-yl)-2-isobutyramidopentanediamide)

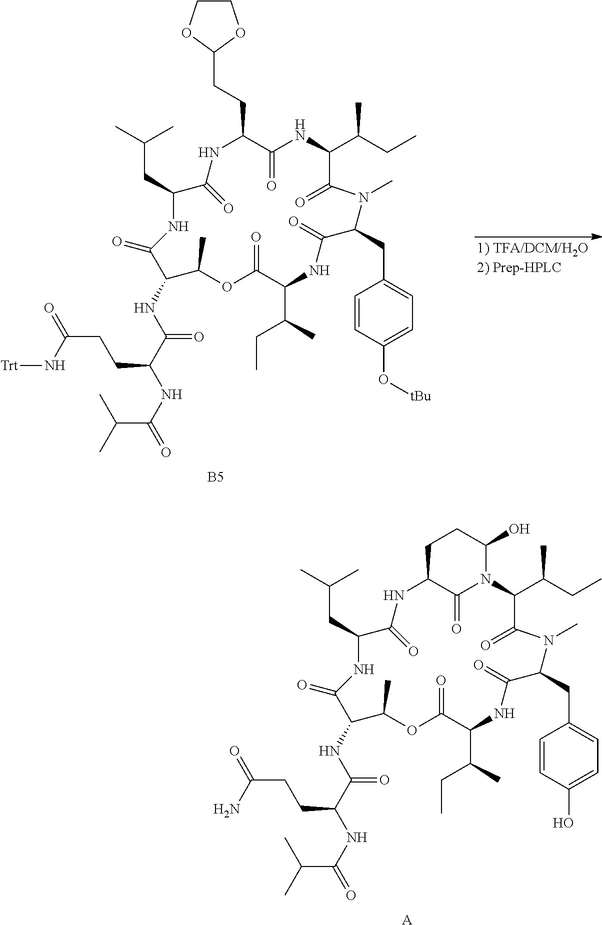

### Solution A

290 ml of DCM and 134 ml of TFA were mixed in a 1.0 l addition funnel.

### Solution B

1.6 g of B5 (1.3 mmol) and 223 ml of DCM were mixed in a 2.0 l RBF. The solution was cooled to 0° C.

Solution A was added dropwise to solution B (over 60 min) and then the reaction mixture was stirred at 0° C. for 4.0 h. 320 ml of DCM and 16 ml of water were added to the reaction mixture. The resulting mixture was stirred for 18 h at rt.

The reaction mixture was washed with a solution of 132 g of sodium acetate in 800 ml of water; the organic layer was kept aside. The aqueous layer was extracted twice with portions of 400 ml of ethyl acetate and the organic layers were pooled. The aqueous layer was discarded. The pooled organic layer was dried with magnesium sulfate, filtered and then the solvent was removed under vacuum at 35° C. until and oily residue was obtained. The residue was added to 400 ml of precooled TBME (0-5° C.). The suspension was stirred at 0-5° C. for 30 min and then solid was separated by filtration using a sintered glass filter (G3), the filter cake was washed with 75 ml cold TBME and dried under vacuum at 35° C. until constant weight to yield 3.2 g of crude A.

The product was purified by prep-HPLC yielding 680 mg of A (0.7 mmol, yield=53.8%) with an HPLC purity of 96.6%.

HR-MS: Calculated for C₄₆H₇₂O₁₂N₈: [M+H]⁺: 929.53425; [M+NH₄]⁺: 946.56080; [M+Na]⁺: 951.51619. Found: [M+H]⁺: 929.53445; [M+NH₄]⁺: 946.56129; [M+Na]⁺: 951.51624.

¹H-NMR (600 MHz, d₆-DMSO) δ_{H}: −0.11 (3H, d, J=6.2 Hz), 0.64 (4H, m), 0.77 (3H, d, J=6.2 Hz), 0.81 (3H, t, J=7.3 Hz), 0.84 (3H, d, J=7.0 Hz), 0.88 (3H, d, J=6.6 Hz), 1.02 (3H, d, J=6.7 Hz), 1.02 (1H, m), 1.03 (3H, d, J=6.7 Hz), 1.09 (1H, m), 1.20 (3H, d, J=6.2 Hz), 1.24 (1H, m), 1.39 (1H, m), 1.51 (1H, m), 1.75 (6H, m), 1.83 (1H. m), 1.92 (1H, m), 2.12 (2H, m), 2.47 (1H, m), 2.58 (1H, m), 2.67 (1H, m), 2.71 (3H, s), 3.16 (1H, d, J=14.2 Hz), 4.30 (1H, m), 4.34 (1H, m), 4.42 (1H, d, J=10.6 Hz), 4.45 (1H, m), 4.61 (1H, d, J=9.2 Hz), 4.71 (1H, dd, J=9.5, 5.5 Hz), 4.93 (1H, s), 5.05 (1H, dd, J=11.4, 2.6 Hz), 5.48 (1H, m), 6.07 (1H, d, J=2.6 Hz), 6.64 (2H, d, J=8.4 Hz), 6.73 (1H, s), 6.99 (2H, d, J=8.4 Hz), 7.25 (1H, s), 7.35 (1H, d, J=9.2 Hz), 7.64 (1H, d, J=9.5 Hz), 7.73 (1H, d, J=9.2 Hz), 8.01 (1H, d, J=7.7 Hz), 8.42 (1H, d, J=8.8 Hz), 9.17 (1H, s).

### Example 2

### Synthesis of Compound A ((S)—N1-((1S,2S,5S,8S,11R,12S,15S,18S,21R)-2,8-di((S)-sec-butyl)-21-hydroxy-5-(4-hydroxybenzyl)-15-isobutyl-4,11-dimethyl-3,6,9,13,16,22-hexaoxo-10-oxa-1,4,7,14,17-pentaazabicyclo[16.3.1]docosan-12-yl)-2-isobutyramidopentanediamide) via on-resin macrolactamization

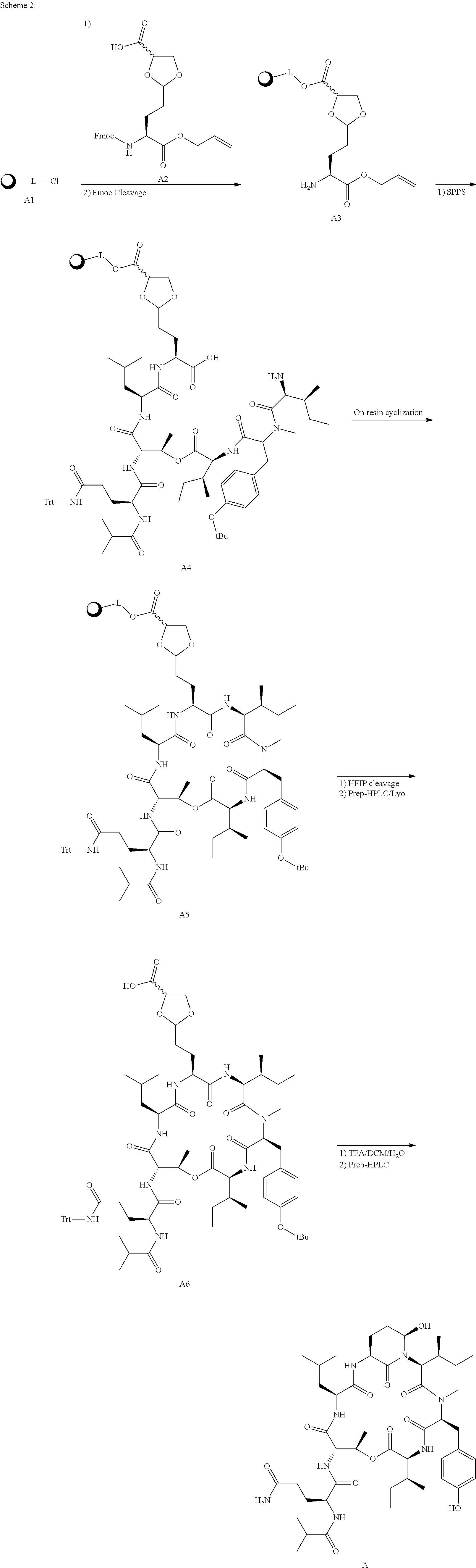

### Example 2.1

### Resin Loading—Synthesis of A3

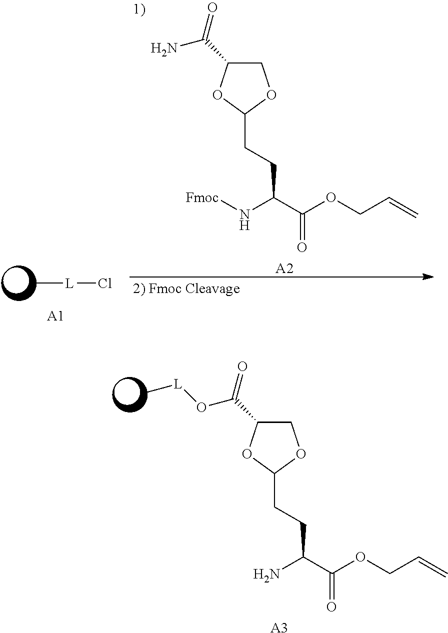

In a 100 ml solid phase synthesis reactor were added 7.0 g of 2-chlorotrityl chloride resin (L=2-chlorotrityl, ‘bead’ (RES)=1.0% divinylbenzene cross-linked polystyrene), A1 (loading=1.6 mmol/g; 11.20 mmol) and 30 ml of DCM. The suspension was stirred for 5 min and then the solvent was drained. The same washing was repeated once. The wet resin was kept aside under nitrogen atmosphere, in the meantime in a RBF were mixed 4.85 g of ((4S)-2-((S)-3-((((9H-fluoren-9-yl)methoxy)carbonyl)amino)-4-(allyloxy)-4-oxobutyl)-1,3-dioxolane-4-carboxylic acid) (A2) (10.08 mmol; 0.96 eq), 21.5 ml of DCM and 11.7 ml of DIPEA (67.20 mmol, 6.0 eq) and the mixture was stirred at rt until a clear solution was obtained. This solution was added in one portion to the solid phase reactor and then the mixture was stirred for 5.0 min and then a second portion of 5.86 ml of DIPEA (33.60 mmol, 3.0 eq) was added to the solid phase reactor. The suspension was stirred for 18 h at rt and then the reaction mixture was drained and the resin was washed twice with 35 ml of a 5% v/v solution of DIPEA in DCM.

Potential unreacted resin sites were quenched by the treatment of the resin with 35 ml of a solution of DCM/MeOH/DIPEA (70/15/15), v/v/v. At this point a sample was taken for loading measurement.

The Fmoc protecting group was cleaved treating the resin with 35 ml of a 25% v/v solution of piperidine in DMF following the general procedure for Fmoc cleavage as described below. The resin was dried under vacuum at 35° C. until constant weight and then 8.9 g of peptide resin A3 were obtained, the loading this resin calculated to be 0.86 mmol/g expressed as free amine.

### Example 2.2

### Solid Phase Peptide Synthesis (SPPS)—Synthesis of A4

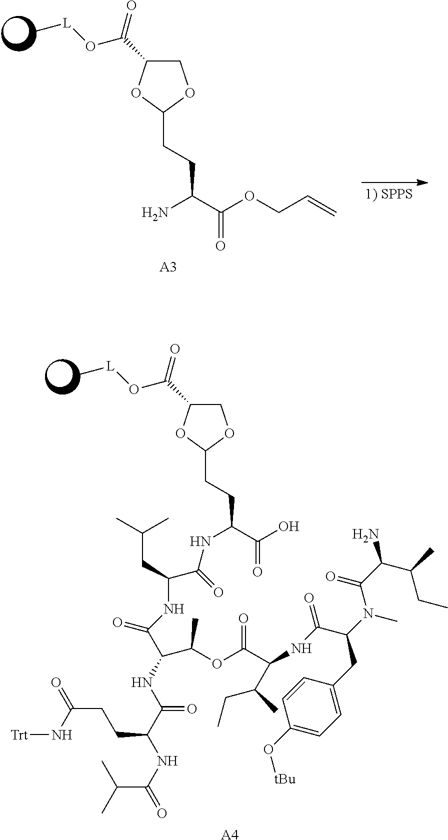

To a 50 ml solid phase peptide synthesis reactor were added 2.3 g of peptide resin A3 (loading=0.86 mmol/g; 5.0 mmols) then the resin was washed with 40 ml of DMF (2×30 min). The resin was subjected to solid phase peptide synthesis using an automatic peptide synthesizer (CSBIO536). The sequence of the peptide was generated with the sequential execution of the cycles* as shown in Table 2 * A cycle is defined by the execution of following 2 sequential operations (In the case of cycle 4 the Fmoc cleavage is not applied due the lack of an Fmoc group).

1. Amino acid coupling (See general procedures below)

2. Fmoc cleavage (See general procedures below)

TABLE 2
Coupling sequence and coupling conditions

| Cycle number | Amino Acid | Coupling Method |
|---|---|---|
| 1 | Fmoc-Leu-OH | HATU/DIPEA |
| 2 | Fmoc-Thr-OH | DICI/Oxyma |
| 3 | Fmoc-Gln(Trt)-OH | DICI/Oxyma |
| 4 | Isobutyric acid | DICI/Oxyma |
| 5 | Fmoc-Ile-OH | MSNT/NMI |
| 6 | Fmoc-MeTyr(tBu)-OH | HATU/DIPEA |
| 7 | Fmoc-Ile-OH | HATU/DIPEA |

After cycle number 7 was finished the peptide resin was washed additionally with 40 ml of isopropanol (3×2 min) and with 40 ml of TMBE (2×2 min).

To the wet resin 20.0 ml of DCM and 1.2 ml of phenylsilane (10.0 mmol, 5.0 eq) were added. The suspension was stirred for 5 minutes at rt while keeping the system under nitrogen atmosphere.

In the meantime 0.23 g of Pd(PPh₃)₄ (0.2 mmol, 0.1 eq), and 10 ml of DCM were mixed in a RBF. The mixture was stirred and added in one portion to the solid phase reactor. The suspension was stirred for 15 min, and the solvent was drained. The process was repeated two more times and then the resin was washed with 40 ml of DCM (4×2 min) and treated with 40 ml of a 0.02 M solution of sodium diethyldithiocarbamate trihydrate in NMP. The suspension was stirred for 10 min and then the reactor was drained.

The Fmoc protecting group was cleaved following the General procedure for Fmoc cleavage as described below. After Fmoc cleavage the resin was washed with 40 ml of IPA (2×2 min) and with 40 ml of TBME (4×2 min). The resin was dried under vacuum at 35° C. until constant weight, yielding 4.98 g of peptide resin A4. This resin was used completely for the next step.

### Example 2.3

### On Resin Cyclization—Synthesis of A5

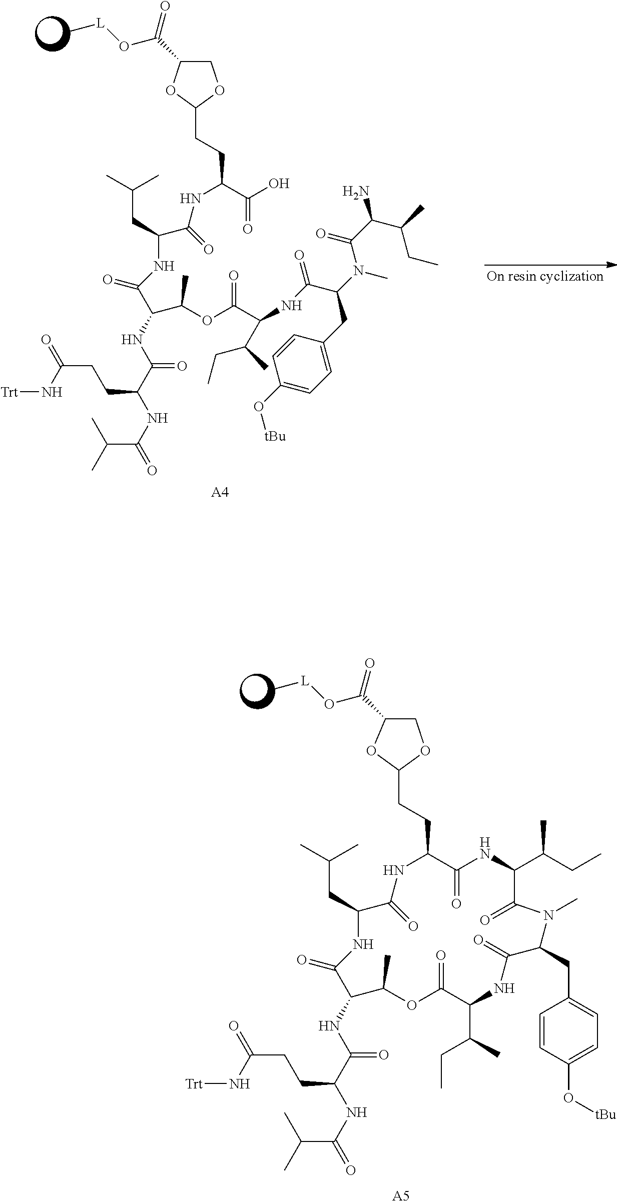

In a RBF 0.4 g of Oxyma (3.0 mmol, 1.5 eq) and 30 ml of DMF were mixed and the mixture was stirred for 2.0 min. The mixture was added to the resin A4 and the suspension was stirred for 5.0 min. Then 0.5 ml of DICI (3.0 mmol, 1.5 eq) were added and the reaction mixture was stirred for 18 h.

The resin was washed with 40 ml of DMF (4×2 min), 40 ml of IPA (2×2 min) and with 40 ml of TBME (4×2 min). The resin was dried under vacuum at 35° C. until constant weight yielding 4.6 g of peptide resin A5.

### Example 2.4

### Cleavage from the resin—Synthesis of A6 ((4S)-2-(2-((3S,6S,9S,12S,15S,18S,19R)-6-(4-(tert-butoxy)benzyl)-3,9-di((S)-sec-butyl)-15-isobutyl-18-((S)-2-isobutyramido-5-oxo-5-(tritylamino)pentanamido)-7,19-dimethyl-2,5,8,11,14,17-hexaoxo-1-oxa-4,7,10,13,16-pentaazacyclononadecan-12-yl)ethyl)-1,3-dioxolane-4-carboxylic acid)

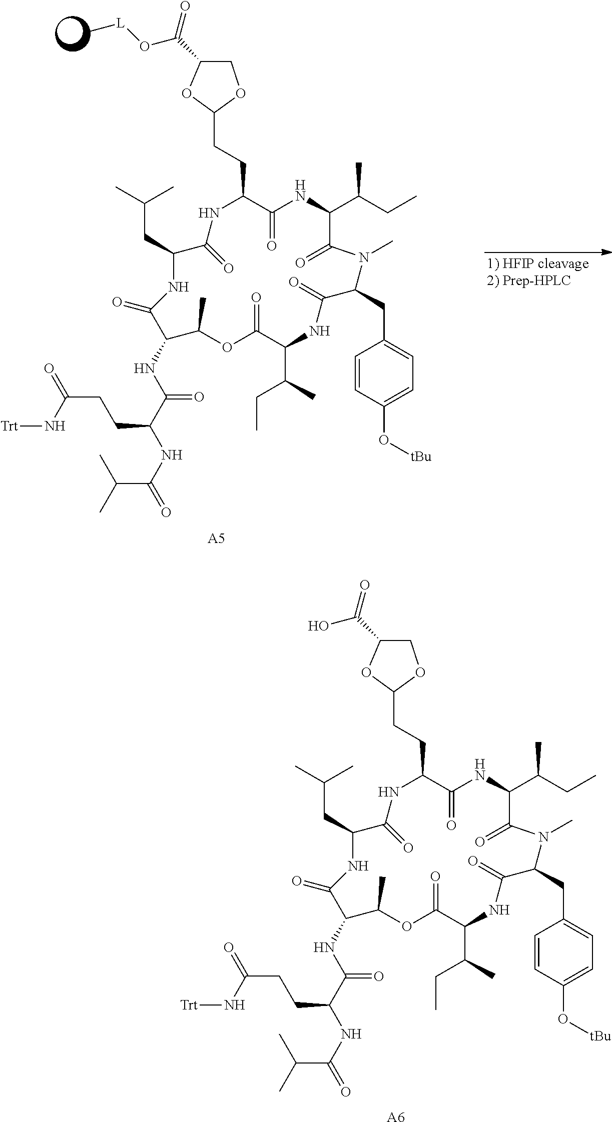

The peptide resin A5 was placed in a 50 ml solid phase reactor and 23 ml of DCM were added. The suspension was stirred for 30 min and then the solvent was drained. The washing was repeated once more and then the solvent was drained.

To the wet resin 23 ml of a 30% HFIP in DCM (v/v), were added in one portion. The suspension was stirred for 10 min and then the solvent was drained and collected in a 500 ml RBF, the cleavage process was repeated twice and the drained solutions were pooled. The pooled solution was concentrated under vacuum at 35° C. until an oily residue was obtained. To this oil 20 ml of toluene were added. The toluene solution was added dropwise to 250 ml of pre-cooled (0-5° C.) heptane, the suspension was stirred for 30 min, filtered with a sintered glass filter (G3) and the filter cake was washed with 25 ml of pre-cooled (0-5° C.) heptane. The wet filter cake was dried under vacuum at 35° C. until constant weight and then 2.6 g of crude peptide A6 were obtained. The product was purified by prep-HPLC to yield 0.27 g (0.21 mmol) of pure A6.

HR-MS: Calculated for C₇₂H₉₈N₈O₁₅: [M−H]⁻: 1313.7073; [M+NH4]+: 1332.7495. Found: [M−H]⁻: 1313.7100; [M+NH₄]⁺: 1332.7495.

¹H-NMR (600 MHz, CDCl₃): δ ppm 0.48 (2H, br, s), 0.75 (7H, br, s), 0.83 (5H, br, s), 1.0 (2H, br, s), 1.10 (3H, br, s), 1.25 (9H, br, s), 1.45 (1H, br, s), 1.53 (4H, br, s), 1.60 (2H, br, s), 1.60 (2H, br, s), 1.63 (4H, br, s), 1.81 (2H, br, s), 2.00 (1H, br, s), 2.31 (2H, br, s), 2.43 (1H, br, s), 2.69 (3H, s), 3.27 (2H, br, s), 3.66 (1H, br, s), 3.79 (1H, br, s), 3.95 (1H, br, s), 4.10 (1H, br, s), 4.18 (1H, d), 4.25 (3H, m), 4.37 (1H, br, s), 5.30 (1H, br, s), 5.30 (1H, br, s), 6.86 (2H, br, s), 7.16 (10H, m), 7.26 (5H, m), 7.82 (1H, br, s), 7.94 (1H, br, s), 8.54 (1H, br, s), 8.74 (1H, br, s), 9.21 (1H, br, s).

### Example 2.5

### Synthesis of A ((S)—N1-((1S,2S,5S,8S,11R,12S,15S,18S,21R)-2,8-di((S)-sec-butyl)-21-hydroxy-5-(4-hydroxybenzyl)-15-isobutyl-4,11-dimethyl-3,6,9,13,16,22-hexaoxo-10-oxa-1,4,7,14,17-pentaazabicyclo[16.3.1]docosan-12-yl)-2-isobutyramidopentanediamide)

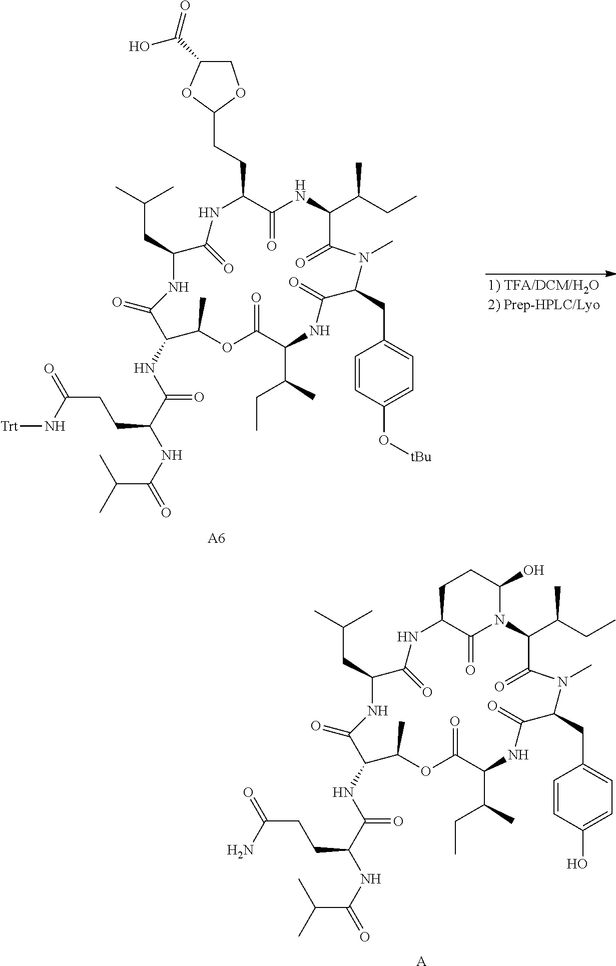

### Solution A′

25 ml of DCM and 14.4 ml of TFA were mixed in a 250 ml erlenmeyer flask and the solution was transferred to an addition funnel.

### Solution B′

0.25 g Peptide A6 (0.190 mmol), 25 ml of DCM were mixed in a 500 mL RBF equipped with a top head stirrer. The solution was cooled to 0° C.

Solution A′ was added dropwise to solution B′ (over 15 min) and then the reaction mixture was stirred at 0° C. for 4.0 h. 50 ml of DCM and 2.5 ml of water were added to the reaction mixture. The resulting mixture was stirred for 18 h at rt.

The reaction mixture was washed with a solution of 20 g of sodium acetate in 100 ml of water; the organic layer was kept aside. The aqueous layer was extracted twice with portions of 50 ml of ethyl acetate and the organic layers were pooled. The aqueous layer was discarded. The pooled organic layer was dried with 50 g of magnesium sulfate, filtered and then the solvent was removed under vacuum at 35° C. until and oily residue was obtained. The residue was dissolved in 3 ml of toluene and added to 25 ml of precooled heptane (0-5° C.). The suspension was stirred at 0-5° C. for 30 min and then solid was separated by filtration using a sintered glass filter (G3), the filter cake was washed with 25 ml cold heptane and dried under vacuum at 35° C. until constant weight to yield 180 mg of crude A.

The product was purified by prep-HPLC yielding 75 mg of A (0.081 mmols, yield=42.5%) with an HPLC purity of 95.2%.

The HR-MS and the NMR data were in agreement with the data of compound A (see Example 1)

### General Procedures:

General Procedure for Amino Acid Coupling Using HATU/DIPEA

The amino acid (15.0 mmol, 3.0 eq) and HATU (15 mmol, 3.0 eq) were dissolved in 30 ml of DMF. To this solution was added 2.1 ml of DIPEA (15.0 mmol, 3.0 eq) and the reaction mixture was stirred for 2-5 min. The reaction mixture was then added to the resin in the solid phase reactor and allowed to react for 2.0 h. The reaction mixture was drained and the resin was washed with 40 ml of DMF (4×2.0 min).

General Procedure for Amino Acid Coupling Using DICI/Oxyma

The amino acid (15.0 mmols, 3.0 eq) and Oxyma (15.0 mmol, 3.0 eq) were dissolved in 30 ml of DMF. To this solution was added DICI (15.0 mmols, 3.0 eq) and the reaction mixture was stirred for 2-5 min. The reaction mixture was then added to the resin in the solid phase reactor and allowed to react for 2.0 h. The reaction mixture was drained and the resin was washed with 40 ml of DMF (4×2.0 min).

General Procedure for Amino Acid Coupling Using MSNT/NMI

In 250 ml RBF and under nitrogen atmosphere were mixed, the amino acid (15.0 mmol, 3.0 eq) MSNT (15.0 mmol, 3.0 eq) and 30 ml of DCM, the suspension was cooled to −10° C., then 2.4 ml of NMI (30.0 mmol, 6.0 eq) was added. The mixture was stirred for 5 min, the solution was added in one portion to the resin in the solid phase reactor, the reaction mixture was stirred for 120 min. The reaction mixture was drained. The resin was washed with 40 ml of DMF (3×2 min).

General Procedure for Fmoc Cleavage

To the resin in the solid phase reactor was added 50 ml of a 25% v/v solution of piperidine in DMF, the suspension was stirred for 5 min and the solvent was drained. Then a second portion of 50 ml of a 25% v/v solution of piperidine in DMF was added and the mixture was stirred for 15 min, the solvent was drained and the resin was washed with 40 ml of DMF (6×2.0 min).

### Synthesis of Intermediates:

Synthesis of B2

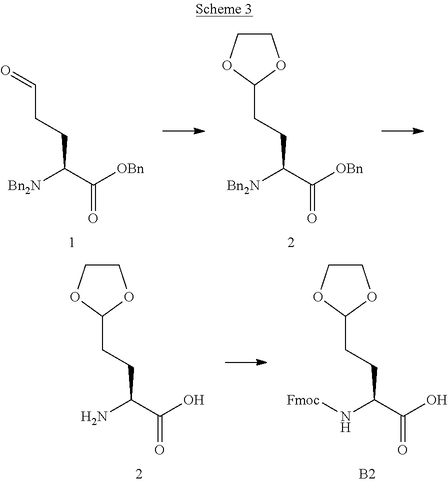

a) Synthesis of Compound 2:

To a solution of compound 1 [Rodriguez and Taddei, Synthesis 2005, 3, pp. 493-495] (29 g; 72.23 mmol) in DCM (700 mL) ethylene glycol (133 g, 2.14 moles), p-toluenesulfonic acid monohydrate (15 g; 78.86 mmol) and molecular sieves (3 Angstrom, 40 g) were sequentially added. The reaction mixture was stirred for 18 h at room temperature. The molecular sieve was removed by filtration, the filter cake was washed with ethyl acetate and the filtrate was evaporated under reduced pressure. The residue was dissolved in ethyl acetate (II), extracted with water (3×300 ml) and the organic phase was evaporated under reduced pressure to obtain 33.3 g crude product. The crude product was purified by chromatography on silica gel with ethyl acetate/hexanes (4:6) to obtain 28.0 g of pure Compound 2 (87% yield).

1H-NMR of the product confirmed the proposed structure.

HR-MS: Calculated for C₂₈H₃₁NO₄ [M+H]+=446.23259. Found: 446.23248.

b) Synthesis of Compound 4:

Compound 4 was prepared by hydrogenation of compound 2 using 10% palladium on charcoal as catalyst under atmospheric hydrogen pressure, in ethanol/water (1:1 v/v) as solvent at room temperature. For work-up, the catalyst was removed by filtration and the solvent was evaporated under reduced pressure. Subsequent drying of the product in vacuo at 45° C. gave compound 4 in quantitative yield.

1H- and 13C-NMR-Spectra confirmed the proposed structure for compound 4.

HR-MS: Calculated for C₇H₁₃NO₄ [M+H]+: 176.09174; [M+Na]+: 198.07368. Found: [M+H]+: 176.09173; [M+Na]+: 198.07362.

c) Synthesis of Compound B2:

Compound 4 (1.2 g; 6.85 mmol) was dissolved in water (7 ml) and triethylamine (0.692 g) was added. To this stirred mixture, a solution of Fmoc-HOSU-Ester (2.31 g; 6.85 mmol) in acetonitrile (6 g) was added and the reaction mixture was stirred for ca. 1 h at rt. The pH value of the resulting reaction mixture was adjusted to 8.5-9.0 by addition of triethylamine in several portions. In total, addition of ca. 0.7 g triethylamine was necessary to maintain a pH of 8.5-9.0. For work-up, the reaction mixture was subjected to flash chromatography on silica gel by direct transfer of the reaction mixture on a silica gel column. Elution with ethyl acetate/acetic acid (98:2), combination of product fractions and evaporation of the solvent gave wet compound B2. The wet product was suspended in hexanes, stirred for 1 h at room temperature and the precipitate was isolated by filtration. The precipitate was dried in vacuo at 50° C. over night to obtain a product comprising ca. 20 mol % of acetic acid. This product was dissolved in ethyl acetate (50 mL) at 60° C. and the solution was cooled down to room temperature. Seed crystals (compound B2) were added at room temperature and the suspension was stirred until a thin suspension was formed. The volume of the suspension was reduced to ca. 15 mL by partial evaporation of the solvent at 40° C. under reduced pressure, and hexanes (89 mL) was added to the suspension over 30 minutes at room temperature. The suspension was stirred for 1 additional h at room temperature and the product was isolated by filtration. The product was dried in vacuo at 50° C. overnight to obtain Compound B2 (2.31 g; 84.85% yield).

HR-MS: Calculated for C₂₂H₂₃NO₆ [M+H]⁺: 398.15982; [M+NH₄]⁺:415.18636; [M+Na]⁺: 420.14176. Found: [M+H]⁺: 398.15991; [M+NH₄]⁺:415.18655; [M+Na]⁺: 420.14183.

¹H-NMR (600 MHz, d₆-DMSO): δ ppm 1.64 (2H, m); 1.68 (1H, m); 1.81 (1H, m); 3.76 (2H, m); 3.87 (2H, m); 3.98 (1H, m); 4.22 (1H, m); 4.27 (2H, m); 4.79 (1H, m); 7.33 (2H, t, J=7.3 Hz); 7.42 (2H, t, J=7.3 Hz); 7.66 (1H, d, J=8.1 Hz); 7.73 (2H, d, broad); 7.89 (2H, d, J=7.3 Hz); 12.59 (1H, s, broad).

¹³C-NMR (150 MHz, d₆-DMSO): δ ppm 25.46 (CH2), 2993 (CH2), 46.67 (CH), 53.61 (CH), 64.27 (2×CH2), 65.65 (CH2), 103.12 (CH), 120.12 (2×CH), 125.30 (2×CH), 127.08 (2×CH), 127.67 (2×CH), 140.71 (2×C), 143.79 (2×C), 156.14 (C), 173.68 (C).

IR: 3345, 3321, 3063, 3021, 2974, 2963, 2949, 2767, 1950, 1914, 1878, 1741, 1691, 1682, 1610, 1541, 1525, 1477, 1464, 1451, 1403, 1367, 1323, 1285, 1270, 1249, 1225, 1188, 1138, 1104, 1087, 1055, 1033, 1008, 983, 963, 939, 925, 873, 836, 798, 782, 759, 740, 648, 622.

Synthesis of A2

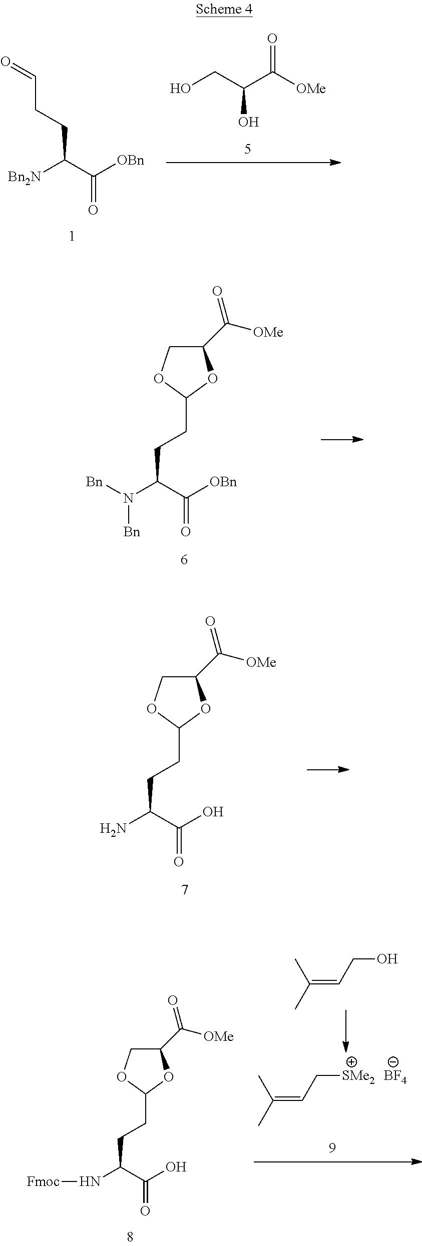

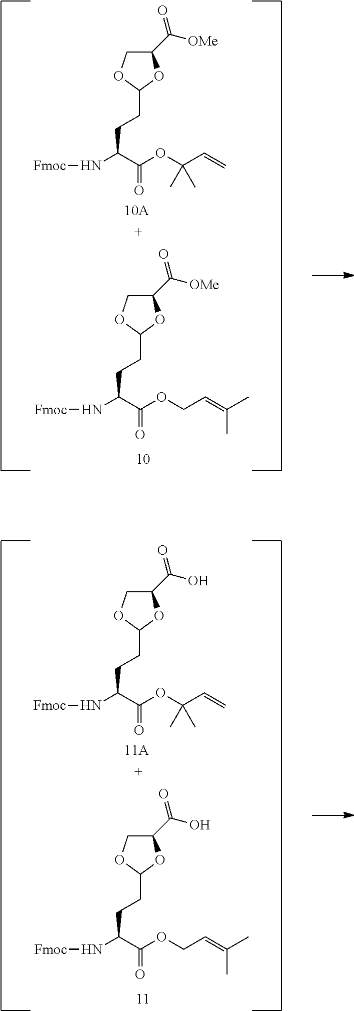

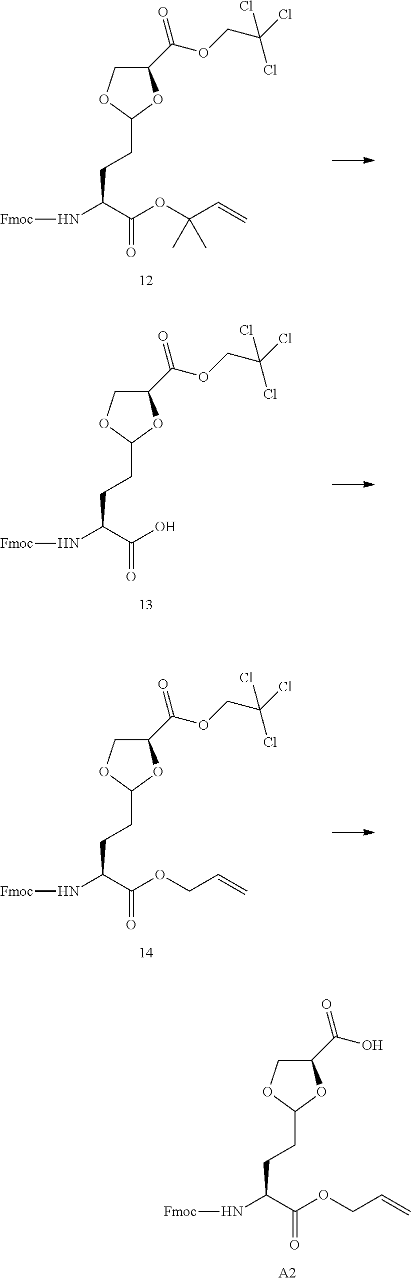

### a) Synthesis of compound 6 (4S)-methyl 2-((S)-4-(benzyloxy)-3-(dibenzylamino)-4-oxobutyl)-1,3-dioxolane-4-carboxylate)

To a solution of compound 1 (47 g, 117 mmol) and (S)-Methyl 2,3-dihydroxypropanoate (14.06 g, 117 mmol) in benzene (1250 ml) was added anhydrous p-toluenesulfonic acid (12.08 g, 70.2 mmol). The resulting reaction mixture was refluxed in Dean-Stark apparatus at 115° C. for 8 h under nitrogen atmosphere. Then the reaction mixture was completely concentrated and co-evaporated with DCM (150 ml×3 times) under reduced pressure to get brownish oil. The crude product was purified by flash column chromatography (silica gel 230-400 mesh, 10-15% ethyl acetate in petroleum ether) to obtain compound 6 (40.1 g, yield 68.02%) as pale yellow oil.

¹H-NMR (400 MHz, CDCl₃), Compound 6: δ ppm 1.48-1.72 (1H, m), 1.85-1.96 (3H, m), 3.38-3.46 (3H, m), 3.70-3.77 (3H, m), 3.87-4.00 (3H, m), 4.12-4.23 (1H, m), 4.45-4.51 (1H, m), 4.82-5.0 (1H, m), 5.13-5.31 (2H, m), 7.22-7.42 (15H, m).

UPLC: Acquity HSS T3 C18 (2.1×50) mm; 1.8 μm; Mobile A: 0.1% HCOOH in Water Mobile B: 0.1% HCOOH in MeCN; Rt 2.03 min; m/z: 526.4 (M+Na⁺), purity 100%.

### b) Synthesis of compound 7 ((2S)-2-amino-4-((4S)-4-(methoxycarbonyl)-1,3-dioxolan-2-yl)butanoic acid)

The reaction has been carried out in 6 parallel batches (each 20 g batch) and work up has been done together.

To a stirred solution of compound 6 (20.0 g, 39.71 mmol) dissolved in methanol (1 l) under nitrogen was added 10% palladium on charcoal (7 g). Reaction flask was evacuated and filled with hydrogen gas and the reaction was stirred under hydrogen atmosphere at room temperature for 16 hours (the reaction was monitored by TLC). All the reaction mixture was collectively filtered through a celite bed, washed with MeOH (10 l) and the filtrate was concentrated under reduced pressure to get compound 7 (46 g, crude) as white solid which was taken as such for next step.

¹H-NMR (400 MHz, d₆-DMSO), Compound 7: δ ppm 1.75-1.83 (4H, m); 3.17 (1H, m); 3.66 (3H, m); 3.79-4.23 (2H, m); 4.60-4.66 (1H, m); 4.88-4.92 (1H, m); 7.67 (br s, 2H). LC-MS m/z: 234 (M+H+), purity 85.2%.

### c) Synthesis of compound 8 (2S)-2-((((9H-fluoren-9-yl)methoxy)carbonyl)amino)-4-((4S)-4-(methoxycarbonyl)-1,3-dioxolan-2-yl)butanoic acid)

To a solution of compound 7 (15 g, 64.32 mmol) in THF-water (1:1 v/v, 300 ml) was added NaHCO₃ (21.6 g, 257.2 mmol) and the mixture was stirred for 10 min. Fmoc-OSu (32.5 g, 96.4 mmol) was added portion wise to the reaction mixture and it was stirred at rt for 16 h. After completion, the reaction mixture was concentrated under reduced pressure, acidified to pH ˜6 using 1.5 N HCl solution at 0° C. The organic product was extracted with ethyl acetate (100 ml×3 times), the combined ethyl acetate extract was dried over anhydrous Na₂SO₄. The organic layer was filtered and concentrated under reduced pressure to get crude product as yellow oil. The crude product was purified by flash column chromatography (silica gel 230-400 mesh, 1-2% methanol in chloroform) to obtain gummy liquid product which was triturated with MTBE to obtain compound 8 (13 g, yield: 44%) as off white solid.

¹H-NMR (300 MHz, d₆-DMSO), Compound 8: δ ppm 1.50-1.96 (4H, m); 3.67 (3H, m); 3.79 (1H, m); 3.96-4.01 (2H, m); 4.20-4.27 (3H, m); 4.60-4.69 (1H, m), 4.93 (1H, m), 7.28-7.42 (4H, m), 7.68-7.72 (2H, m), 7.86-7.88 (2H, m), 12.58 (br s, 1H)

### d) Synthesis of compound 9 (dimethyl(3-methylbut-2-en-1-yl)sulfonium tetrafluoroborate)

To a cooled solution (−20° C.) of 3-methyl-2-buten-1-ol (15 g, 174.1 mmol) in anhydrous dichloromethane (200 mL) was added dimethyl sulphide (128 ml, 1741.5 mmol) under nitrogen atmosphere. After 10 min, tetrafluoroboric acid diethyl ether complex (11.16 ml, 82.05 mmol) was added and the reaction mixture was stirred at 0° C. for 6 hours followed by rt for 14 h. The solvent was removed under reduced pressure and the brown liquid residue was dissolved with acetonitrile (50 ml). The resultant solution was washed with saturated sodium bicarbonate solution until it reached to pH ˜7. The organic layer was dried over anhydrous sodium sulphate and the solvent was concentrated under reduced pressure. Product 9 (16 g, yield 42.14%) was obtained as off-white solid which was taken as such for next step without further purification.

¹H-NMR (400 MHz, D₂O), Compound 9: δ ppm 1.67 (3H, s), 1.77 (3H, s), 2.65 (6H, s), 3.87 (2H, m), 5.19 (1H, m).

### e) Synthesis of compound 10/10A ((4S)-methyl 2-((S)-3-((((9H-fluoren-9-yl)methoxy)carbonyl)amino)-4-((3-methylbut-2-en-1-yl)oxy)-4-oxobutyl)-1,3-dioxolane-4-carboxylate/(4S)-methyl 2-((S)-3-((((9H-fluoren-9-yl)methoxy)carbonyl)amino)-4-((2-methylbut-3-en-2-yl)oxy)-4-oxobutyl)-1,3-dioxolane-4-carboxylate)

The reaction has been carried out in 2 parallel batches (each in 80 g scale).

To a stirred suspension of compound 8 (80 g, 175.6 mmol), potassium carbonate (29.12 g, 210.72 mmol), and copper(I) bromide (1 g, 7.02 mmol) in DCM (1.1 l) was added drop wise a solution of compound 9 (38.3 g, 175.6 mmol) in DCM (500 ml), resulting reaction mixture was stirred at rt for 16 h under nitrogen atmosphere (monitored by TLC). After completion, both the reaction mixtures were collectively filtered through a celite bed and the clear filtrate was concentrated to afford colorless residue. The crude product was purified by flash column chromatography (silica gel 230-400 mesh, 10-15% ethyl acetate in petroleum ether) to obtained mixture of regioisomers 10/10A (142 g, yield 77.2%) as pale yellow oil.

¹H-NMR (400 MHz, d₆-DMSO), Mixture of isomers 10/10A: δ ppm 1.44 (6H, s, [isomer 10]), 1.63-1.88 (6H [isomer 10A]+4H [isomer 10+10A], m), 3.66 (3H [isomer 10+10A], m), 3.81 (1H [isomer 10+10A], m), 3.96-4.13 (2H [isomer 10+10A], m), 4.21-4.29 (2H [isomer 10A]+2H [isomer 10+10A], m), 4.5-4.7 (1H+1H [isomer 10+10A], m), 4.94 (1H [isomer 10+10A], m), 5.01-5.18 (2H [isomer 10], m), 5.26 (1H [isomer 10A], m), 6.01 (1H [isomer 10], m), 7.30-7.34 (2H [isomer 10+10A], m), 7.39-7.43 (2H [isomer 10+10A], m), 7.72 (2H [isomer 10+10A], m), 7.89 (2H [isomer 10+10A], m).

UPLC: Acquity HSS T3 C18 (2.1×50) mm; 1.8 μm; Mobile A: 0.1% HCOOH in Water Mobile B: 0.1% HCOOH in MeCN; Rt 1.80 min; m/z: 524.6 (M+H+), purity 97.8%.

### f) Synthesis of compound 11/11A ((4S)-2-((S)-3-((((9H-fluoren-9-yl)methoxy)carbonyl)amino)-4-((3-methylbut-2-en-1-yl)oxy)-4-oxobutyl)-1,3-dioxolane-4-carboxylic acid/(4S)-2-((S)-3-((((9H-fluoren-9-yl)methoxy)carbonyl)amino)-4-((2-methylbut-3-en-2-yl)oxy)-4-oxobutyl)-1,3-dioxolane-4-carboxylic acid)

To a stirred solution of compound 10/10A (10.2 g, 19.48 mmol) in dichloroethane (200 ml) was added trimethyltin hydroxide (4.93 g, 27.2 mmol) and the reaction mixture was stirred at rt for 16 h under nitrogen atmosphere. The reaction mixture was concentrated under reduced pressure and co-evaporated with CH₂Cl₂ (2 times). The resulting crude product was purified by flash chromatography (silica gel 230-400 mesh, 30-100% ethyl acetate in petroleum ether) to obtain 11/11A (mixture of regioisomers) (6.1 g, 60.42%) as pale yellow viscous oil.

¹H-NMR (400 MHz, d₆-DMSO), Mixture of isomers 11/11A: δ ppm 1.44 (6H, s, [isomer 165]), 1.63-1.89 (6H [isomer 165A]+4H [isomer 165+165A], m), 3.76-4.03 (2H+1H [isomer 165+165A], m), 4.19-4.27 (2H [isomer 165A]+2H [isomer 165+165A], m), 4.5 (1H+1H [isomer 165+165A], m), 4.94 (1H [isomer 165+165A], m), 5.01-5.19 (2H [isomer 165], m), 5.26 (1H [isomer 165A], m), 5.99 (1H [isomer 165], m), 7.30-7.34 (2H, m), 7.39-7.43 (2H, m), 7.72 (2H, m), 7.89 (2H, m).

LC-MS m/z: 508.4 (M−H+), purity 99.6%.

UPLC: Acquity HSS T3 C18 (2.1×50) mm; 1.8 μm; Mobile A: 0.1% HCOOH in Water Mobile B: 0.1% HCOOH in MeCN; isomer 1: Rt 1.61, isomer 2: Rt 1.62 min; m/z: 532.3 (M+Na⁺), purity 59.08+40.92%.

### g) Synthesis of compound 12 ((4S)-2,2,2-trichloroethyl 2-((S)-3-((((9H-fluoren-9-yl)methoxy)carbonyl)amino)-4-((2-methylbut-3-en-2-yl)oxy)-4-oxobutyl)-1,3-dioxolane-4-carboxylate)

56.35 g of compound 11/11A (110.6 mmol, 1.0 eq), 24.8 g of 2,2,2 trichloroethanol (165.9 mmol, 1.5 eq) were dissolved in 500 ml of DCM and then 13.9 g of DICI (110.6 mmol, 1.0 eq) and 1.35 g of DMAP (11.06 mmol, 0.1 eq) were added. The reaction was stirred at rt for 30 min. The reaction mixture was filtered and the organic phase was extracted with NaHCO₃ sat (3×150 mL), 10% critric acid (1×150 mL) and brine (1×150 mL). The organic phase was dried over MgSO₄, filtered and concentrated to an oil containing some precipitated urea. The urea was removed by dissolving the compound in TBME and filtering the solution.

The compound was used for the next step without further purification.

### h) Synthesis of compound 13 ((2S)-2-((((9H-fluoren-9-yl)methoxy)carbonyl)amino)-4-((4S)-4-((2,2,2-trichloroethoxy)carbonyl)-1,3-dioxolan-2-yl)butanoic acid)

20.9 g of compound 12 (32.6 mmol, 1.0 eq), and 10.6 g of phenylsilane (97.8 mmol, 3.0 eq) were dissolved in DCM and then 1.8 g of Pd(PPh₃)₄ (1.6 mmol, 0.05 eq), the reaction mixture was stirred at r.t and followed by HPLC. The solvent was removed under vacuum and the crude product was purified using by silica gel filtration.

Gradient from Heptane:EtOAc (95:5) to Heptane:EtOAc (60:40). The fraction containing the compound were collected together and concentrated under vacuum to yield compound 13.

### i) Synthesis of compound 14 ((4S)-2,2,2-trichloroethyl 2-((S)-3-((((9H-fluoren-9-yl)methoxy)carbonyl)amino)-4-(allyloxy)-4-oxobutyl)-1,3-dioxolane-4-carboxylate)

13.6 g of compound 13 (23.7 mmol, 1.0 eq), 100 ml of allyl alcohol, then 3.3 g of DICI (26.1 mmol, 1.1 eq) and 0.3 g of DMAP (2.4 mmol, 0.1 eq) were added. The reaction was stirred at rt and followed by HPLC. The solvent was removed under vacuum and the crude product was dissolved in 200 ml of EtOAc, washed with a saturated solution of NaHCO₃ sat (3×50 mL), 10% citric acid (1×50 mL) and Brine (1×50 mL). The organic phase was dried over MgSO₄, filtered and concentrated under vacuum to get an oil. The crude was purified by silical gel chromatography using a gradient from Heptane:EtOAc (95:5) to Heptane:EtOAc (60:40). The compound came out at 40% EtOAc in heptane. All fractions containing the compound were collected together and concentrated under vacuum to yield compound 14.

### Synthesis of compound A2 ((4S)-2-((S)-3-((((9H-fluoren-9-yl)methoxy)carbonyl)amino)-4-(allyloxy)-4-oxobutyl)-1,3-dioxolane-4-carboxylic acid)

11.2 g of compound 14 (18.3 mmol, 1.0 eq) were dissolved in 100 mL of THF, 5.6 g of zinc (91.4 mmol, 5.0 eq) were added followed by 10 mL of 1.0 N NH₄OAc was added. The reaction mixture was stirred for 10 min and then filtered through a Celite pad. The pad was washed with 30 mL of THF. The filtrate was concentrated under vacuum. The crude product was dissolved in 50 mL of EtOAc and the organic phase was washed with a solution of 10% citric acid (2×20 mL). The organic phase was dried ever MgSO₄, filtered and concentrated to an oil that was purified by flash chromatography using a gradient from heptane:EtOAc (20:80) to (50:50) to yield 3.1 g of compound A2.

HR-MS: Calculated for C₂₆H₂₇NO₈: [M+H]⁺: 482.1815. Found: [M+H]⁺: 482.1817, 499.2080[M+NH₄]⁺: 504.1628[M+Na]⁺.

¹H-NMR (600 MHz, d₆-DMSO): δ ppm 1.89 (1H, m); 4.05 (1H, m); 4.14 (1H, m); 4.24 (2H, m); 4.31 (2H, m); 4.54 (1H, dd); 4.60 (2H, br, s); 4.97 (1H, m); 5.21 (1H, d); 5.31 (1H, m); 5.91 (1H, m); 7.35 (2H, m); 7.43 (2H, m); 7.75 (2H, m); 7.89 (3H, m).

1. A process for the preparation of a cyclic depsipeptide compound of formula (I), or a salt thereof, 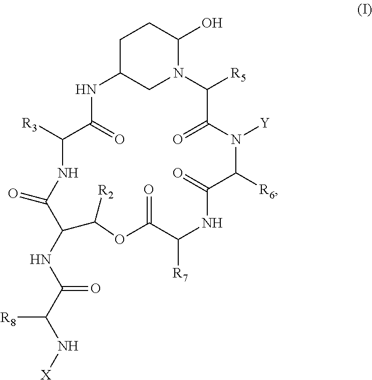
wherein
X is C₁₋₉-acyl;
R₂ is C₁₋₈-alkyl;
R₃ is the side chain of an alpha-amino acid;
R₅ is the side chain of an alpha-amino acid;
R₆ is the side chain of an alpha-amino acid, wherein the side chain contains a hydroxy group;
R₇ is the side chain of an alpha-amino acid;
R₈ is the side chain of an alpha-amino acid, wherein the side chain contains a terminal carboxy or carbamoyl group; and
Y is hydrogen or C₁₋₈-alkyl;
said process comprising
submitting compound of formula (II), or a salt thereof, 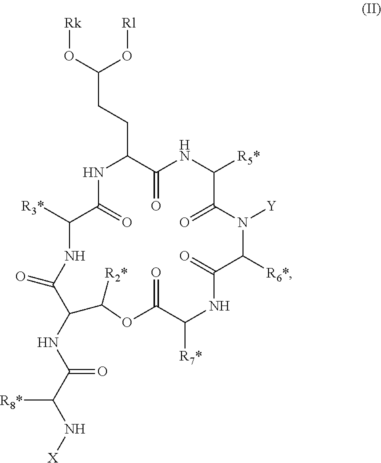
wherein the Rk and Rl are independently of each other linear or branched C₁₋₈-alkyl or benzyl or, Rk and Rl together form a linear or branched C₁₋₈-alkylene bridge, so that Rk and Rl together with the two oxygen atoms and the carbon atom to which the two oxygen atoms are bound, form a 5-7 membered ring; Y and X are as defined for a compound of formula (I) and R₂*, R₃*, R₅*, R₆*, R₇* and R₈* correspond to R₂, R₃, R₅, R₆, R₇ and R₈ in formula (I), respectively, but with the proviso that reactive functional groups on these residues are present in protected form, if they could participate in undesired side reactions, to acetal deprotecting conditions.

2-4. (canceled)

5. A process for the preparation of a cyclic depsipeptide compound of formula (I), or a salt thereof, according to claim 1, further comprising for the synthesis of a compound of formula (II), or a salt thereof, 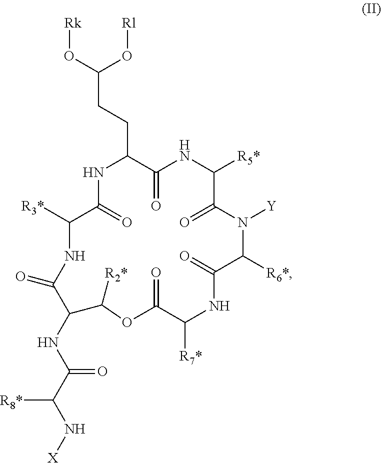
wherein Y and X are as defined for a compound of formula (I) in claim 1 and Rk, Rl, R₂*, R₃*, R₅*, R₆*, R₇* and R₈* are as defined for a compound of formula (II) in claim 1, a process comprising
submitting a linear precursor peptide is of the formula (III) or a salt thereof, 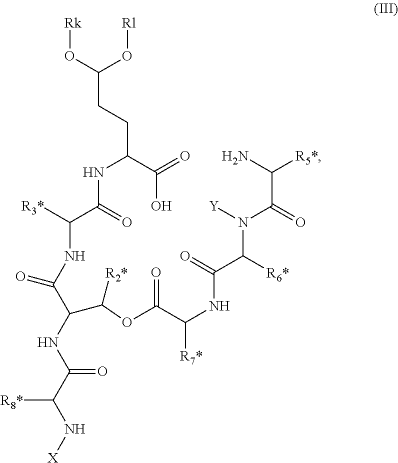
wherein Y and X are as defined for a compound of formula (I) in claim 1 and Rk, Rl, R₂*, R₃*, R₅*, R₆*, R₇* and R₈* are as defined for a compound of formula (II) in claim 1, to macrolactamization conditions.

6. A process for the preparation of a cyclic depsipeptide compound of formula (I), or a salt thereof, according to claim 5, further comprising for the synthesis of a compound of formula (III), or a salt thereof, 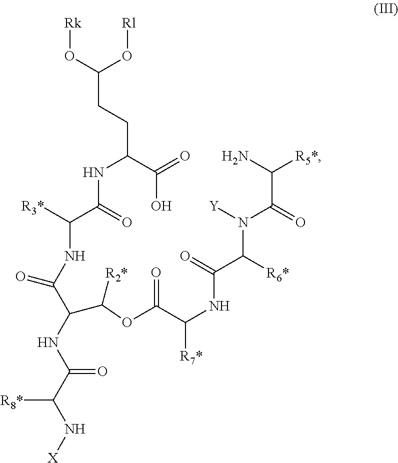
wherein Y and X are as defined for a compound of formula (I) in claim 1 and Rk, Rl, R₂*, R₃*, R₅*, R₆*, R₇* and R₈* are as defined for a compound of formula (II) in claim 1, a process comprising
submitting a compound of formula (IV), 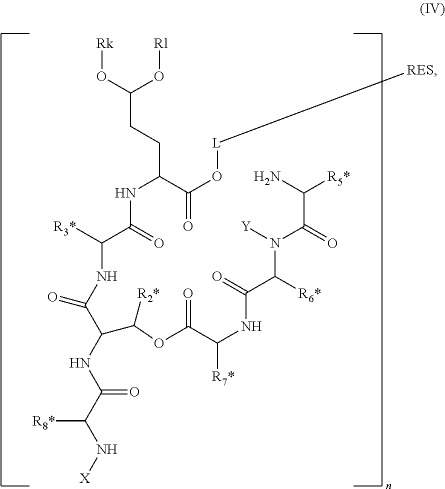
wherein Y and X are as defined for a compound of formula (I) in claim 1 and Rk, Rl, R₂*, R₃*, R₅*, R₆*, R₇* and R₈* are as defined for a compound of formula (II) claim 1,
L is a cleavable linker, RES is a solid resin and n is a natural number not including 0, to cleavage conditions.

7. A process for the preparation of a cyclic depsipeptide compound of formula (I), or a salt thereof, according to claim 6, further comprising for the synthesis of a compound of formula (IV), 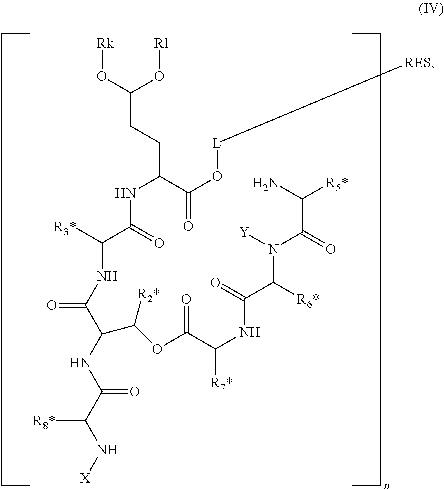
wherein Y and X are as defined for a compound of formula (I) in claim 1 and Rk, Rl, R₂*, R₃*, R₅*, R₆*, R₇* and R₈* are as defined for a compound of formula (II) in claim 1, L is a cleavable linker, RES is a solid resin and n is a natural number not including 0,
a process comprising
submitting a compound of formula (XVI) 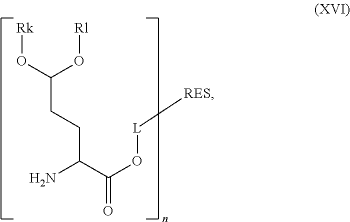
wherein Rk and Rl are as defined for a compound of formula (II) in claim 1,
L is a cleavable linker, RES is a solid resin, and n is a natural number not including 0, to Solid Phase Peptide Synthesis (SPPS).

8. A process for the preparation of a cyclic depsipeptide compound of formula (I), or a salt thereof, 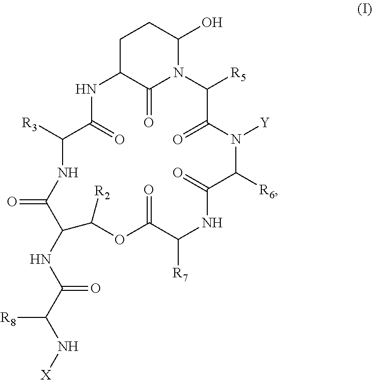
wherein
X is C₁₋₉-acyl;
R₂ is C₁₋₈-alkyl;
R₃ is the side chain of an alpha-amino acid;
R₅ is the side chain of an alpha-amino acid;
R₆ is the side chain of an alpha-amino acid, wherein the side chain contains a hydroxy group;
R₇ is the side chain of an alpha-amino acid;
R₈ is the side chain of an alpha-amino acid, wherein the side chain contains a terminal carboxy or carbamoyl group; and
Y is hydrogen or C₁₋₈-alkyl;
said process comprising
submitting compound of formula (II′), or a salt thereof, 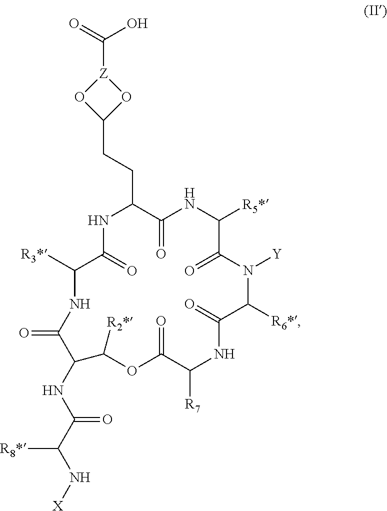
wherein Z is a linear or branched C₂₋₈-alkylene bridge, where Z together with the two oxygen atoms and the carbon atom to which the two oxygen atoms are bound, form a 5-7 membered ring, Y and X are as defined for a compound of formula (I) and R₂*′, R₃*′, R₅*′, R₆*′, R₇*′ and R₈*′ correspond to R₂, R₃, R₅, R₆, R₇ and R₈ in formula (I), respectively, but with the proviso that reactive functional groups on these residues are present in protected form, if they could participate in undesired side reactions,
to acetal deprotecting conditions.

9-12. (canceled)

13. A process for the preparation of a cyclic depsipeptide compound of formula (I), or a salt thereof, according to claim 8, further comprising for the synthesis of a compound of formula (II′), or a salt thereof, 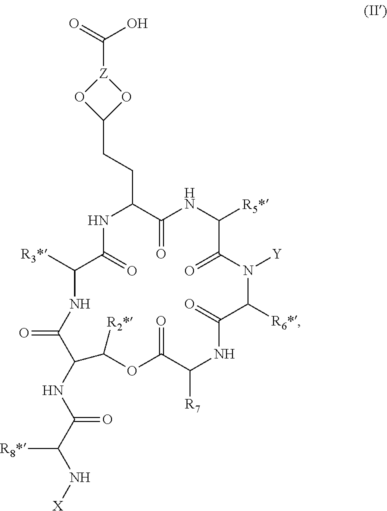
wherein Y and X are as defined for a compound of formula (I) in claim 8 and Rk, Rl, R₂*, R₃*, R₅*, R₆*, R₇* and R₈* are as defined for a compound of formula (II′) in claim 8,
a process comprising
submitting a compound of formula (III′) 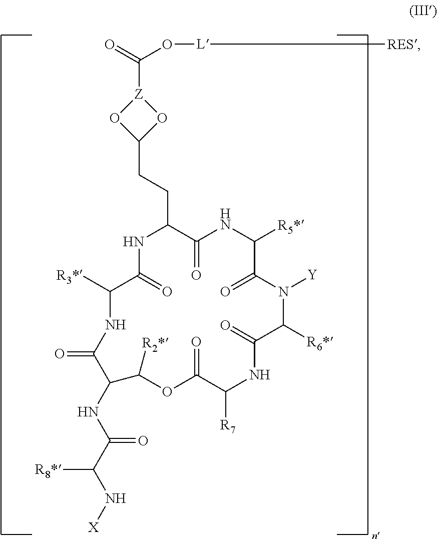
wherein Y and X are as defined for a compound of formula (I) in claim 8 and Z, R₂*′, R₃*′, R₅*′, R₆*′, R₇*′ and R₈*′ are as defined for a compound of formula (II′) in claim 8,
L′ is a cleavable linker, RES′ is a solid resin and n′ is a natural number not including 0, to cleavage conditions.

14. A process for the preparation of a cyclic depsipeptide compound of formula (I), or a salt thereof, according to claim 13, further comprising for the synthesis of a compound of formula (III′), 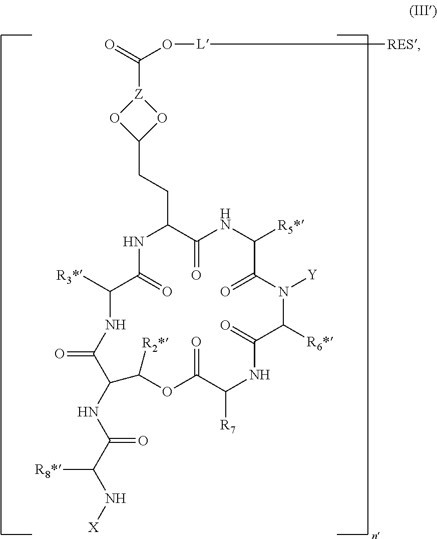
wherein Y and X are as defined for a compound of formula (I) in claim 8 and Z, R₂*′, R₃*′, R₅*′, R₆*′, R₇*′ and R₈*′ are as defined for a compound of formula (II′) in claim 8,
L′ is a cleavable linker, RES′ is a solid resin and n′ is a natural number not including 0,
a process comprising
submitting a compound of formula (IV′), 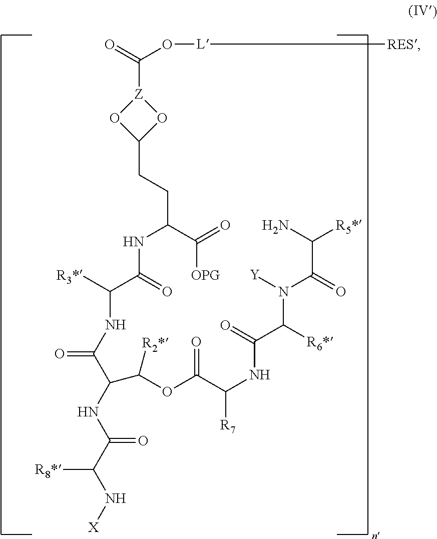
wherein Y and X are as defined for a compound of formula (I) in claim 8 and Z, R₂*′, R₃*′, R₅*′, R₆*′, R₇*′ and R₈*′ are as defined for a compound of formula (II′) in claim 8,
L′ is a cleavable linker, RES′ is a solid resin, n′ is a natural number not including 0 and PG is a carboxy protecting group,
to macrolactamization conditions.

15. A process for the preparation of a cyclic depsipeptide compound of formula (I), or a salt thereof, according to claim 14, further comprising for the synthesis of a compound of formula (IV′), 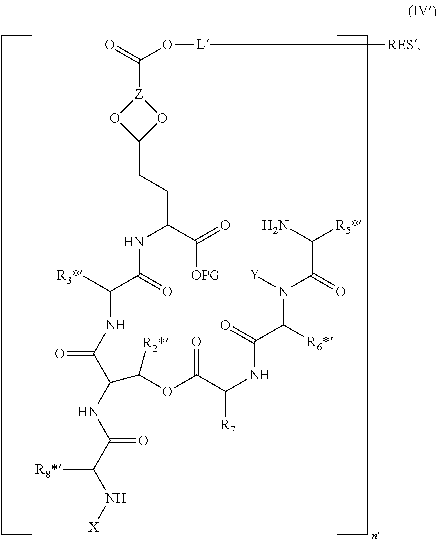
wherein Y and X are as defined for a compound of formula (I) in claim 8 and Z, R₂*′, R₃*′, R₅*′, R₆*′, R₇*′ and R₈*′ are as defined for a compound of formula (II′) in claim 8,
L′ is a cleavable linker, RES′ is a solid resin, n′ is a natural number not including 0 and PG is a carboxy protecting group,
a process comprising
submitting a compound of formula (XVI′) 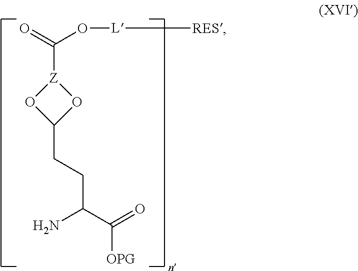
wherein Z is as defined for a compound of formula (II′) in claim 8,
L′ is a cleavable linker, RES′ is a solid resin, n′ is a natural number not including 0 and PG is a carboxy protecting group,
to Solid Phase Peptide Synthesis (SPPS).

16. A process for the preparation of a cyclic depsipeptide compound of formula (I), or a salt thereof, according to claim 15, further comprising for the synthesis of a compound of formula (XVI′), 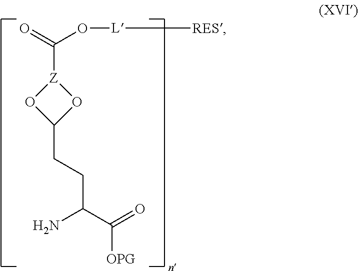
wherein Z is as defined for a compound of formula (II′) in claim 8,
L′ is a cleavable linker, RES′ is a solid resin, n′ is a natural number not including 0 and PG is a carboxy protecting group,
a process comprising
submitting a formula (XVII′), 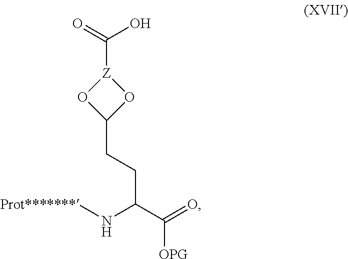
wherein Z is as defined for a compound of formula (II′) in claim 8, PG is a carboxy protecting group and Prot*******′ is an amino protecting group,
to conditions for loading to solid support.

17. A compound of formula (III), 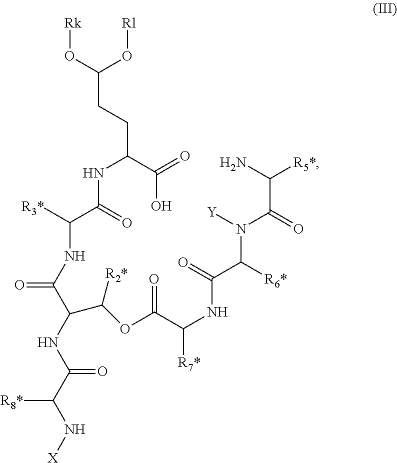
or
a compound of formula (II′): 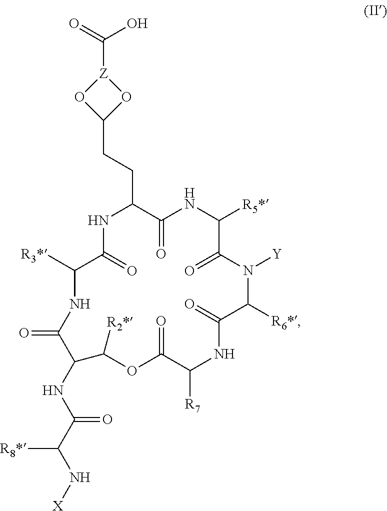
or a compound of formula (XVII′), 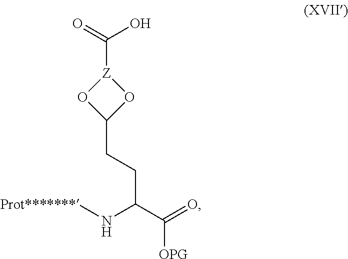
wherein Z is a linear or branched C₂₋₈-alkylene bridge, where Z together with the two oxygen atoms and the carbon atom to which the two oxygen atoms are bound, form a 5-7 membered ring,
PG is a carboxy protecting group and Prot*******′ is an amino protecting group
and wherein X is C₁₋₉-acyl;
Y is hydrogen or C₁₋₈-alkyl;
Rk and Rl are independently of each other linear or branched C₁₋₈-alkyl or benzyl or, Rk and Rl together form a linear or branched C₁₋₈-alkylene bridge, so that Rk and Rl together with the two oxygen atoms and the carbon atom to which the two oxygen atoms are bound, form a 5-7 membered ring;
R₂* is C₁₋₈-alkyl;
R₃* is the side chain of an alpha-amino acid;
R₅* is the side chain of an alpha-amino acid;
R₆* is the side chain of an alpha-amino acid, wherein the side chain contains a hydroxy group;
R₇* is the side chain of an alpha-amino acid;
R₈* is the side chain of an alpha-amino acid, wherein the side chain contains a terminal carboxy or carbamoyl group; with the proviso that reactive functional groups on R₂*, R₃*, R₅*, R₆*, R₇* and R₈* are present in protected form, if they could participate in undesired side reactions.

18-19. (canceled)
